(19) United States
(12) Patent Application Publication
(10) Pub. No.: US 2025/0258146 A1
(43) Pub. Date: Aug. 14, 2025

(54) QUANTITATIVE DETECTION METHOD OF MULTIPLE METABOLITES IN BIOLOGICAL SAMPLE AND METABOLIC CHIP

(71) Applicant: HUMAN METABOLOMICS INSTITUTE INC., Shenzhen (CN)

(72) Inventors: Wei Jia, Shenzhen (CN); Guoxiang Xie, Shenzhen (CN); Lu Wang, Shenzhen (CN)

(21) Appl. No.: 19/169,027

(22) Filed: Apr. 3, 2025

Related U.S. Application Data
(63) Continuation-in-part of application No. 17/309,062, filed on Jul. 17, 2021.

(30) Foreign Application Priority Data
Oct. 19, 2018 (CN) 201811223486.X

(51) Int. Cl. G01N 30/72; G01N 30/02; G01N 30/04; G01N 30/06; G01N 30/14; G01N 30/88; G01N 33/487

(52) U.S. Cl. CPC G01N 30/7266; G01N 33/487; G01N 2030/027; G01N 2030/045; G01N 2030/062; G01N 2030/067; G01N 2030/146; G01N 2030/8813; G01N 2030/8818; G01N 2458/15

(57) ABSTRACT

The present invention discloses a quantitative detection method of multiple metabolic components in a biological sample and a metabolic chip used in the method. The detection method includes performing derivatization treatment on the biological sample and then detecting the derivatized biological sample by liquid chromatography-mass spectrometry. The metabolic chip of the present invention includes a chip carrier microtiter plate and related reagents, and quantitative detection of multiple metabolic components of different magnitudes such as amino acid, phenol, phenyl or benzyl derivative, indole, organic acid, fatty acid, sugar, and bile acid in the biological sample on the same microtiter plate can be achieved.

### CROSS-REFERENCE TO RELATED APPLICATIONS

The present application is a Continuation-in-part Application of U.S. patent application Ser. No. 17/309,062 filed on Jul. 17, 2021, which is U.S. national stage application of International Application No. PCT/CN2019/112389, filed on Oct. 21, 2019, which claims the right of the priority of Chinese patent application 201811223486.X filed on Oct. 19, 2018. The contents of the above Chinese patent application are incorporated herein by reference in their entireties.

### TECHNICAL FIELD

The present invention relates to the field of detection of biological samples, specifically relates to a quantitative determination method of multiple metabolic components such as amino acid, phenol, phenyl or benzyl derivative, indole, organic acid, fatty acid, sugar, and bile acid in a biological sample, and more specifically relates to a detection method of a biological sample by using chemical derivatization and tandem mass spectrometry and a metabolic chip used in the method.

### BACKGROUND

Metabolomics involves unbiased analysis of all metabolites (metabolomes) in cells, body fluids and tissues. At present, with a metabolomics platform based on nuclear magnetic resonance (NMR) or mass spectrometry (MS), many small molecule (MW<1500) metabolites are detected, but only relative (non-absolute) concentrations of the metabolites in biological fluids (serum/plasma or urine) and tissues of subjects suffering from metabolic diseases are provided to determine that the concentrations are different from those of a control group. Due to high chemical diversity of the metabolomes, there is a great challenge to full-spectrum quantitative detection of these metabolites. Since a quantitative metabolomics platform for large-scale biological sample analysis is in deficiency, clinical practicality and application of metabolomics have not yet been realized.

Quantitative metabolomics is used for identifying and quantifying as many metabolites as possible in a biological sample. Compared with traditional targeted and non-targeted methods, quantitative metabolomics has many advantages, including lowest cross-platform variability, improved stability and maintenance of full-spectrum metabolic characteristics and more detailed information about the identity and concentration of specific metabolites.

With regard to quantification of metabolite concentration, reliable analytical data is a prerequisite for development of metabolic-based clinical trials or a thorough understanding of functions of organisms and biological systems in translational researches.

One of the technical challenges is that concentrations of metabolites are in a range of more than a dozen of magnitudes. For example, glucose in blood is in some compounds such as eicosanoids in a millimolar range and a femtomolar range. There are also different platform challenges in size and polarity differences of compounds. In order to overcome these challenges, gas chromatography-mass spectrometry (GC-MS) and liquid chromatography-mass spectrometry (LC-MS) have been applied to a maximum coverage. However, the difficulty in quantitative detection of multiple indexes at the same time has not yet been solved.

There are only a few major metabolic pathways, such as glycolysis, aerobic respiration, tricarboxylic acid (TCA) cycle, fatty acid oxidation (0-oxidation) and gluconeogenesis. Cells are used to transfer energy and maintain metabolic homeostasis, and due to defects in these key pathways in storage and disposal of major classes of molecules (such as amino acid, carbohydrate, and lipid), metabolic disorders are caused. Therefore, unique characteristics of metabolites in biological systems can be reflected in quantitative detection of these metabolites.

As important substances involved in physiological metabolism of the human body, amino acid, phenol, phenyl or benzyl derivative, indole, organic acid, fatty acid, sugar, and bile acid are maintained at a certain level in normal metabolism in the human body. Generally, the changes of concentrations of these substances indicate that there are abnormalities in metabolic pathways of the human body. Based on detection of concentrations of these substances and clinical manifestations, judgment of clinical diseases is facilitated.

In view of the shortcomings of the prior art, an objective of the present invention is to provide a metabolic chip capable of quantitatively detecting multiple components in a biological sample at the same time and a detection method.

### BRIEF SUMMARY OF THE INVENTION

An objective of the present invention is to provide a quantitative detection method capable of quantitatively detecting multiple metabolic components in a biological sample at the same time and a metabolic chip used in the method. The multiple metabolic components include, but are not limited to, multiple amino acids, phenols, phenyl or benzyl derivatives, indoles, organic acids, fatty acids, sugars, and bile acids.

In the present invention, the quantitative detection method of multiple metabolic components in a biological sample is achieved by using the following solution: derivatization treatment is performed on the biological sample, and then the derivatized biological sample is detected by liquid chromatography-mass spectrometry; during derivatization treatment, 3-nitrophenylhydrazine is used as a derivatization reagent, and 1-(3-dimethylaminopropyl)-3-ethylcarbodiimide is used as a derivatization reaction catalyst; the biological sample is selected from urine, blood, cerebrospinal fluid, tissue, cell, saliva and fecal samples of a mammal; the multiple metabolic components in the biological sample are selected from one or more of amino acid, phenol, phenyl or benzyl derivative, indole, organic acid, fatty acid, sugar, and bile acid and have different magnitudes in content.

In the present invention, the quantitative detection method of multiple metabolic components in a biological sample is achieved by using the following solution: derivatization treatment is performed on the biological sample, and then the derivatized biological sample is detected by liquid chromatography-mass spectrometry; during derivatization treatment, 3-nitrophenylhydrazine is used as a derivatization reagent, and 1-(3-dimethylaminopropyl)-3-ethylcarbodiimide is used as a derivatization reaction catalyst; the biological sample is selected from urine, blood, cerebrospinal fluid, tissue, cell, saliva and fecal samples of a mammal; the multiple metabolic components in the biological sample detected by the quantitative detection method are selected from amino acid, phenol, phenyl or benzyl derivative, indole, organic acid, fatty acid, sugar, and bile acid and have different magnitudes in content.

In some embodiments, the liquid chromatography adopts a UPLC BEH C18 chromatographic column, the mobile phase includes: A: water containing 0.1% formic acid, B: acetonitrile containing 0.1% formic acid and isopropanol at a ratio of 1-2:1; and the gradient elution conditions: 0-1 min, 5% B; 1-5 min, 5-30% B; 5-9 min, 30-50% B; 9-12 min, 50-75% B; 12-15 min, 75-95% B; 15-16 min, 95-100% B; 16-18 min, 100% B; and 18-20 min, 5% B.

In some embodiments, the liquid chromatography adopts a UPLC BEH C18 chromatographic column, the mobile phase includes: A: water containing 0.1% formic acid, B: acetonitrile containing 0.1% formic acid and isopropanol at a ratio of 1-2:1; the flow rate is 0.4 mL/min; and the gradient elution conditions: 0-1 min, 5% B; 1-5 min, 5-30% B; 5-9 min, 30-50% B; 9-12 min, 50-75% B; 12-15 min, 75-95% B; 15-16 min, 95-100% B; 16-18 min, 100% B; and 18-20 min, 5% B.

In some embodiments, the liquid chromatography adopts a UPLC BEH C18 chromatographic column, the mobile phase includes: A: water containing 0.1% formic acid, B: acetonitrile containing 0.1% formic acid and isopropanol at a ratio of 1-2:1; the flow rate is 0.4 mL/min; the injection volume is 5 microliters; and the gradient elution conditions: 0-1 min, 5% B; 1-5 min, 5-30% B; 5-9 min, 30-50% B; 9-12 min, 50-75% B; 12-15 min, 75-95% B; 15-16 min, 95-100% B; 16-18 min, 100% B; and 18-20 min, 5% B.

In some embodiments, the liquid chromatography adopts a UPLC BEH C18 chromatographic column, the mobile phase includes: A: water containing 0.1% formic acid, B: acetonitrile containing 0.1% formic acid and isopropanol at a ratio of 1-2:1; the column temperature is 40° C.; the flow rate is 0.4 mL/min; the injection volume is 5 microliters; and the gradient elution conditions: 0-1 min, 5% B; 1-5 min, 5-30% B; 5-9 min, 30-50% B; 9-12 min, 50-75% B; 12-15 min, 75-95% B; 15-16 min, 95-100% B; 16-18 min, 100% B; and 18-20 min, 5% B.

In some embodiments, the mobile phase includes: A: water containing 0.1% formic acid, B: acetonitrile containing 0.1% formic acid and isopropanol at a ratio of 7:3.

In some embodiments, scan parameters of the mass spectrometry are set as follows: S-lens RF level, 50; mass range, 100 to 1200 m/z; full MS resolution, 70,000; MS/MS resolution, 17,500; and NCE/stepped NCE is set at 10, 20, and 40 eV.

In some embodiments, ESI ion source parameters of the mass spectrometry are set as follows: spray voltage, 3800 V; sheath gas flow rate, 40 for ESI+ and 5 for ESI−; capillary temperature, 320° C.; probe heater temperature, 350° C.; aux gas flow rate, 10.

In some embodiments, the liquid chromatography-mass spectrometry is acquired using a Vanquish Flex UHPLC system coupled to a Q Extractive Focus Orbitrap mass spectrometer equipped with a heated electrospray ionization (ESI) source.

In some embodiments, the number of the multiple metabolic components in a biological sample detected by the quantitative detection method is 50 or more, preferably 70 or more, further preferably 90 or more.

In some embodiments, the number of the metabolic components in a biological sample detected by the quantitative detection method is 80 or more, preferably 90 or more, further preferably 100 or more.

In some embodiments, the number of the metabolic components in a biological sample detected by the quantitative detection method is 200 or more, preferably 300 or more, further preferably 400 or more.

In some embodiments, the number of the multiple metabolic components in a biological sample detected by the quantitative detection method is 50 or more, preferably 100 or more, more preferably 200 or more, further preferably 600.

In some embodiments, the number of the metabolic components in a biological sample detected by the quantitative detection method is 1000 or more.

In some embodiments, the multiple metabolic components of the biological sample detected by the quantitative detection method are selected from the following multiple metabolites: fructose 1,6-diphosphate, 10Z-nonadecenoic acid, trans-li-octadecenoic acid, cis-11-octadecenoic acid, 12-dehydrocholic acid, 12-hydroxystearic acid, 12-ketolithocholic acid, 1-methylhistidine, 2,2-dimethylsuccinic acid, 2,3-diaminopropionic acid, glucoside 24-chenodeoxycholic acid, 2-butenoic acid, 2-oxoadipic acid, 2-methyl-4-pentenoic acid, 2-methyl-β-alanine, 2-methylbutyric acid, 2-methylhexanoic acid, 2-methylglutaric acid, 2-methylglutamic acid, 2-furoic acid, 2-hydroxy-2-methylbutyric acid, 2-hydroxy-3-methylbutyric acid, 2-hydroxybutyric acid, 2-hydroxycaproic acid, 2-hydroxycinnamic acid, 2-hydroxyphenylacetic acid, 2-hydroxyhippuric acid, 2-phenylpropionic acid, 2-phenylglycine, 3-(3-hydroxyphenyl)-3-hydroxypropionic acid, 3-(4-hydroxyphenyl)lactic acid, 3,4-dihydroxymandelic acid, 3,4-dihydroxyphenylpropionic acid, 3,4-dehydro-DL-proline, 3,5-diiodo-L-thyroxine, 3β-cholic acid, 3-pyridineacetic acid, 3-indoleacrylic acid, 3-indolepropionic acid, 3-indoleacetamide, 3-oxyalanine, 3-aminosalicylic acid, 3-chloro-L-tyrosine, 3-methyl-2-oxobutyric acid, 3-methyl-2-oxovaleric acid, 3-methylindole, 3-methyladipic acid, 3-methylglutamic acid, 3-nitrotyrosine, sulfate 3-taurolithocholic acid, sulfate 3-lithocholic acid, 3-hydroxy-2-aminobenzoic acid, 3-hydroxybutyric acid, 3-hydroxypropionic acid, 3-hydroxyisovaleric acid, 3-hydroxyphenylacetic acid, 3-hydroxyhippuric acid, 3-dehydrocholic acid, 3-phenylbutyric acid, 4-methyl-2-oxovaleric acid, 4-methylhexanoic acid, 4-methoxyphenylacetic acid, 4-hydroxy-3-methoxymandelic acid, 4-hydroxycinnamic acid, 4-hydroxybenzoic acid, 4-hydroxyhippuric acid, 4-hydroxyphenyllactic acid, 4-hydroxyphenylpyruvic acid, 4-phenylbutyric acid, 5-aminolevulinic acid, 5-hydroxytryptophan, 5-hydroxytryptamine, 5-hydroxylysine, 6,7-diketolithocholic acid, 6-phosphogluconic acid, 6-ketolithocholic acid, 7,12-diketolithocholic acid, 7-ketolithocholic acid, 7-ketodeoxycholic acid, 9,11-conjugated linoleic acid, D-2-hydroxyglutaric acid, D-galactose, D-xylose, D-xylulose, D-fructose, D-ribose, D-ribose-5-phosphate, D-ribulose, D-ribulose-5-phosphate, D-mannose, D-glucose, D-maltose, L-2-aminobutyric acid, L-3-phenyllactic acid, L-alanine, L-serine, L-acetylcarnitine, L-lactic acid, L-allothreonine, L-cysteine, L-homoserine, L-homocysteine, L-homocitrulline, L-pipecolic acid, L-aspartic acid, L-asparagine, L-sorbose, L-lignic acid, L-norleucine, L-kynurenine, L-thyronine, L-arginine, L-histidine, L-valine, L-cystine, L-cystathionine, L-proline, L-tryptophan, L-threonine, L-phenylalanine, L-malic acid, L-methionine, L-glutamine, L-glutamic acid, L-lysine, L-tyrosine, L-arabinose, N-(3-phenylpropionyl)glycine, N-acetyl-L-alanine, N-acetyl-L-aspartic acid, N-acetyl-L-phenylalanine, N-acetyl-L-methionine, N-acetyl-L-tyrosine, N-acetylserine, N-acetyl-D-glucosamine, N-acetyl-L-phenylalanine, N-acetylmannosamine, N-acetylneuraminic acid, N-acetylhistidine, N-acetylhydroxytryptamine, N-acetyltryptophan, N-acetylglutamine, N-acetyllysine, N-acetylornithine, N-methylnicotinamide, N-phenylacetyl-glutamine, N-phenylacetylphenylalanine, S-adenosine homocysteine, α-D-glucose, α-lactose, α-linolenic acid, α-hydroxyisobutyric acid, α-ketoglutaric acid, α-muricholic acid, β-D-trehalose, β-alanine, β-ursocholic acid, β-muricholic acid, γ-L-glutamyl-L-alanine, γ-linolenic acid, γ-aminobutyric acid, ω-muricholic acid, butyric acid, trimethylamine nitroxide, adenosine triphosphate, malonic acid, propionic acid, acetoacetic acid, acetylcysteine, acetylglycine, acetylornithine, acetic acid, guanidine acetate, glycolic acid, lactulose, lactoylglutathione, heneicosanoic acid, cis-12-heneicosenoic acid, heptacosanoic acid, tricosanoic acid, cis-14-tricosenoic acid, docosanoic acid, docosatrienoic acid, cis-13,16-docosadienoic acid, docosapentaenoic acid, docosahexaenoic acid, docosatetraenoic acid, trans-13-docosaenoic acid, cis-13-docosaenoic acid, pentacosanoic acid, octacosanoic acid, hexacosanoic acid, tetracosanoic acid, eicosanoic acid, eicosatrienoic acid, eicosadienoic acid, eicosapentaenoic acid, trans-II-eicosenoic acid, cis-ii-eicosenoic acid, cis-5-eicosenoic acid, cis-8-eicosenoic acid, dimethylglycine, adenosine diphosphate, linoleic acid, leucine, alloisoleucine, allolithocholic acid, allocholic acid, undecenoic acid, heptadecanoic acid, tridecanoic acid, nonadecanoic acid, nonadecadienoic acid, pentadecanoic acid, galactonic acid, galactitol, mecysteine, protocatechuic acid, apocholic acid, dehydrolithocholic acid, nordeoxycholic acid, trans-9-tetradecenoic acid, trans-aconitic acid, trans-4-hydroxyproline, trans-9-heptadecenoic acid, trans-9-pentadecenoic acid, trans-9-hexadecenoic acid, trans-linolenic acid, trans-cinnamic acid, elaidic acid, homocysteine, pyrrole-2-carboxylic acid, picolinic acid, indole, indole-3-methyl acetate, indole-3-carboxylic acid, indoleacetic acid, purine, azelaic acid, nonanoic acid, dopa, dopamine, melibiose, fumaric acid, acetaminophen, p-aminohippuric acid, p-cresol sulfate, symmetrical dimethylarginine, p-hydroxymandelic acid, p-hydroxyphenylacetic acid, homogentisic acid, adipic acid, pimelic acid, isobutyric acid, isoleucine, isovaleric acid, citric acid, isoursodeoxycholic acid, isohyodeoxycholic acid, isolithocholic acid, isocholic acid, isodeoxycholic acid, glutaric acid, glutaconic acid, valeric acid, mandelic acid, lauric acid, fructose-6-phosphate, citraconic acid, citric acid, citramalic acid, ribonolactone, ribonic acid, raffinose, palmitoleic acid, palmitic acid, norvaline, n-hydroxyphenylacetic acid, oxidized glutathione, aminoadipic acid, aminocaproic acid, salicyluric acid, oleic acid, trehalose, nicotinic acid, pyroglutamic acid, ursocholic acid, ursodeoxycholic acid, tauro-a-muricholic acid, tauro-b-muricholic acid, tauro-w-muricholic acid, tauroursodeoxycholic acid, taurohyocholic acid, taurohyodeoxycholic acid, taurolithocholic acid, taurocholic acid, taurodehydrocholic acid, taurochenodeoxycholic acid, hyocholic acid, hyodeoxycholic acid, succinylacetone, succinic acid, citrulline, glycodehydrocholic acid, glycolithocholic acid, glycodeoxycholic acid, glycine, glycochenodeoxycholic acid, glyceraldehyde, glyceraldehyde-3-phosphate, choline glycerophosphate, glycoursodeoxycholic acid, glycohyocholic acid, glycohyodeoxycholic acid, glycocholic acid, glyproline, glycyl-L-leucine, mannose-6-phosphoric acid, mannitol, methylmalonic acid, methylsuccinic acid, formic acid, capric acid, lithocholic acid, selenomethionine, thiamine, glycolithocholic sulfate, stearic acid, liothyronine, dihydroxyacetone phosphate, phosphoribosyl pyrophosphate, creatine phosphate, hydroxypyruvic acid, glycine hydroxyphenylacetate, cinnamic acid, sarcosine, carnosine, creatine, inositol, epinephrine, choline, cholic acid, dehydrocholic acid, demethylcholic acid, adenosine monophosphate, arachidonic acid, phenylpyruvic acid, phenethylamine, phenylpropionic acid, phenyllactic acid, benzamide, benzoic acid, oxalic acid, shikimic acid, glucaric acid, glucose-6-phosphate, glucose lactone, sebacic acid, ricinoleic acid, sucrose, methionine sulfoxide, itaconic acid, melatonin, glutathione, myristic acid, erythronic acid, erythrose, suberic acid, caprylic acid, phthalic acid, tartaric acid, tyramine, quinic acid, m-aminobenzoic acid, asymmetric dimethylarginine, tannic acid, cis-10,12-octadecadienoic acid, cis-12,15-heneicosadienoic acid, cis-12-tridecenoic acid, cis-15-tetracosenic acid, cis-2-hydroxycinnamic acid, cis-9-tetradecenoic acid, cis-10-heptadecenoic acid, cis-11-dodecenoic acid, cis-4-hydroxyproline, cis-5-dodecenoic acid, cis-7-hexadecenoic acid, cis-9-heptadecenoic acid, cis-aconitic acid, vanillic acid, hippuric acid, maleic acid, homovanillic acid, ornithine, guanosine monophosphate, guanosine triphosphate, anserine, chenodeoxycholic acid, maltotriose, maltitol, rhamnose and murideoxycholic acid.

The detection method of the present invention specifically includes the following steps:
a) collecting a biological sample;
b) extracting the biological sample with a mixed solvent of methanol, chloroform, and water, performing centrifugation, and taking a supernatant, namely a biological sample extract;
c) adding the same volume of a 3-nitrophenylhydrazine methanol solution and a 1-(3-dimethylaminopropyl)-3-ethylcarbodiimide pyridine solution into the biological sample extract obtained in b), and performing uniform vortex mixing and heating for derivatization, where the concentration of used 3-nitrophenylhydrazine is 100-320 mmol/L (in this patent application, “mM” represents “mmol/L”), the concentration of 1-(3-dimethylaminopropyl)-3-ethylcarbodiimide is 50-200 mmol/L, the reaction temperature is 20-60° C., and the reaction time is 10-120 minutes;
d) adding a carbon-13 labeled isotope internal standard solution obtained from the reaction of 3-nitrophenylhydrazine and 1-(3-dimethylaminopropyl)-3-ethylcarbodiimide into the derivatized biological sample extract obtained in c); and
e) adding a methanol-water mixed solution into a sample in d) for dilution, and determining amino acid, phenol, phenyl or benzyl derivative, indole, organic acid, fatty acid, sugar, and bile acid by liquid chromatography-mass spectrometry.

Preferably, in step c), the concentration of used 3-nitrophenylhydrazine is 150-220 mM, the concentration of 1-(3-dimethylaminopropyl)-3-ethylcarbodiimide is 80-120 mM, the reaction temperature is 20-40° C., and the reaction time is 30-60 minutes.

Preferably, the volume ratio of methanol to water in step e) is 1:1.

When the biological sample is urine, blood, saliva or cerebrospinal fluid, a treatment method of the biological sample in step b) preferably includes: taking an appropriate amount of the biological sample, extracting the biological sample with a mixed solvent of cold methanol, chloroform and water at a volume ratio of 3:1:1, shaking the mixture for a few seconds, performing centrifugation on the sample at a rotation speed of 10000-20000 rpm at a low temperature for 5-15 minutes, and transferring a supernatant into an autosampler glass vial for subsequent derivatization determination.

When the biological sample is a fecal sample, a treatment method of the biological sample in step b) preferably includes: freeze-drying the fecal sample; uniformly mixing an appropriate amount of the freeze-dried fecal sample and an appropriate amount of a mixed solvent of cold methanol, chloroform and water at a volume ratio of 3:1:1, performing centrifugation on the sample at a rotation speed of 10000-20000 rpm at a low temperature for 15-30 minutes, and transferring a supernatant into an autosampler glass vial for subsequent derivatization treatment.

When the biological sample is a tissue or cell sample, a treatment method of the biological sample in step b) preferably includes: adding an appropriate amount of a mixed solvent of cold methanol, chloroform, and water at a volume ratio of 3:1:1 into the sample for homogenizing, performing centrifugation on the sample at a rotation speed of 10000-20000 rpm at 4° C. for 15-30 minutes, and transferring a supernatant into an autosampler glass vial for subsequent derivatization treatment.

According to the detection method provided in the present invention, 3-nitrophenylhydrazine is used to undergo a derivatization reaction with amino acid, phenol, phenyl or benzyl derivative, indole, organic acid, fatty acid, sugar, and bile acid in the sample in the presence of 1-(3-dimethylaminopropyl)-3-ethylcarbodiimide to produce corresponding derivatives, the detection sensitivity is improved, the detection difficulty is reduced, quantitative detection of multiple substances of different magnitudes in the biological sample can be achieved, defects in the prior art are overcome, and a high-throughput quantitative detection effect is achieved. In addition, in the present invention, a commonly labeled derivatization reagent and a carbon-13 labeled derivatization reagent are used to undergo a reaction with the standard product and the sample solution so that chromatographic behaviors, ionization efficiency of mass spectrometry and matrix effect can be completely consistent, and thus systematic errors are avoided.

The present invention provides a quantitative detection method of multiple metabolic components of different magnitudes in a biological sample. As understood by a person of ordinary skill, the content of index components in the sample can be calculated by drawing a standard curve in the present invention. On this basis, the applicant further provides a metabolic chip used in the detection method. The metabolic chip includes

The present invention further provides a metabolic chip used in the detection method. The metabolic chip of the present invention is a device for efficient quantitative detection of multiple metabolic components by using the detection method of the present invention, including a chip carrier, a filter device and dry solid powder of a standard product and a quality control product. The chip carrier is a microtiter plate, and the microtiter plate may be a commercially available 48-well plate, a 96-well plate, and a 384-well microtiter plate suitable for liquid chromatography determination. Each well of the microtiter plate is provided with an independent filter device, and the filter device is a filter membrane made of polyvinylidene fluoride, cellulose acetate, or nylon with a pore size of 0.20-0.45 micron (m). Each well of the microtiter plate is divided into upper and lower parts by the filter device.

The dry solid powder of the standard product and the quality control product is powder obtained by dehydrating or freeze-drying solutions of the standard product and the quality control product and is placed on the filter device in each well of the microtiter plate. As understood by a person of ordinary skill in the art, when the powder of the standard product is prepared, different standard product solutions are prepared first according to required standard product concentration gradients based on the drawn standard curve, then dehydrating or freeze-drying is performed to obtain the powder, and the powder is placed on the corresponding filter devices in the wells of the metabolic chip. As understood by a person of ordinary skill in the art, the quality control product is prepared into a corresponding solution and then dehydrated or freeze-dried to obtain the powder, and the powder is placed on the filter device in each well of the metabolic chip.

The standard product is selected from one or more of amino acid, phenol, phenyl or benzyl derivative, indole, organic acid, fatty acid, sugar, and bile acid standard products. The quality control product is selected from one or more of amino acid, phenol, phenyl or benzyl derivative, indole, organic acid, fatty acid, sugar, and bile acid standard products corresponding to the standard products above.

The multiple metabolic components in the standard product are selected from amino acid, phenol, phenyl or benzyl derivative, indole, organic acid, fatty acid, sugar, and bile acid standard products. The multiple metabolic components in the quality control product are selected from amino acid, phenol, phenyl or benzyl derivative, indole, organic acid, fatty acid, sugar, and bile acid standard products corresponding to the standard products above.

Specifically, the standard product and the quality control product may be selected from the following components: fructose 1,6-diphosphate, 10Z-nonadecenoic acid, trans-11-octadecenoic acid, cis-11-octadecenoic acid, 12-dehydrocholic acid, 12-hydroxystearic acid, 12-ketolithocholic acid, 1-methylhistidine, 2,2-dimethylsuccinic acid, 2,3-diaminopropionic acid, glucoside 24-chenodeoxycholic acid, 2-butenoic acid, 2-oxoadipic acid, 2-methyl-4-pentenoic acid, 2-methyl-β-alanine, 2-methylbutyric acid, 2-methylhexanoic acid, 2-methylglutaric acid, 2-methylglutamic acid, 2-furoic acid, 2-hydroxy-2-methylbutyric acid, 2-hydroxy-3-methylbutyric acid, 2-hydroxybutyric acid, 2-hydroxycaproic acid, 2-hydroxycinnamic acid, 2-hydroxyphenylacetic acid, 2-hydroxyhippuric acid, 2-phenylpropionic acid, 2-phenylglycine, 3-(3-hydroxyphenyl)-3-hydroxypropionic acid, 3-(4-hydroxyphenyl)lactic acid, 3,4-dihydroxymandelic acid, 3,4-dihydroxyphenylpropionic acid, 3,4-dehydro-DL-proline, 3,5-diiodo-L-thyroxine, 30-cholic acid, 3-pyridineacetic acid, 3-indoleacrylic acid, 3-indolepropionic acid, 3-indoleacetamide, 3-oxyalanine, 3-aminosalicylic acid, 3-chloro-L-tyrosine, 3-methyl-2-oxobutyric acid, 3-methyl-2-oxovaleric acid, 3-methylindole, 3-methyladipic acid, 3-methylglutamic acid, 3-nitrotyrosine, sulfate 3-taurolithocholic acid, sulfate 3-lithocholic acid, 3-hydroxy-2-aminobenzoic acid, 3-hydroxybutyric acid, 3-hydroxypropionic acid, 3-hydroxyisovaleric acid, 3-hydroxyphenylacetic acid, 3-hydroxyhippuric acid, 3-dehydrocholic acid, 3-phenylbutyric acid, 4-methyl-2-oxovaleric acid, 4-methylhexanoic acid, 4-methoxyphenylacetic acid, 4-hydroxy-3-methoxymandelic acid, 4-hydroxycinnamic acid, 4-hydroxybenzoic acid, 4-hydroxyhippuric acid, 4-hydroxyphenyllactic acid, 4-hydroxyphenylpyruvic acid, 4-phenylbutyric acid, 5-aminolevulinic acid, 5-hydroxytryptophan, 5-hydroxytryptamine, 5-hydroxylysine, 6,7-diketolithocholic acid, 6-phosphogluconic acid, 6-ketolithocholic acid, 7,12-diketolithocholic acid, 7-ketolithocholic acid, 7-ketodeoxycholic acid, 9,11-conjugated linoleic acid, D-2-hydroxyglutaric acid, D-galactose, D-xylose, D-xylulose, D-fructose, D-ribose, D-ribose-5-phosphate, D-ribulose, D-ribulose-5-phosphate, D-mannose, D-glucose, D-maltose, L-2-aminobutyric acid, L-3-phenyllactic acid, L-alanine, L-serine, L-acetylcarnitine, L-lactic acid, L-allothreonine, L-cysteine, L-homoserine, L-homocysteine, L-homocitrulline, L-pipecolic acid, L-aspartic acid, L-asparagine, L-sorbose, L-lignic acid, L-norleucine, L-kynurenine, L-thyronine, L-arginine, L-histidine, L-valine, L-cystine, L-cystathionine, L-proline, L-tryptophan, L-threonine, L-phenylalanine, L-malic acid, L-methionine, L-glutamine, L-glutamic acid, L-lysine, L-tyrosine, L-arabinose, N-(3-phenylpropionyl)glycine, N-acetyl-L-alanine, N-acetyl-L-aspartic acid, N-acetyl-L-phenylalanine, N-acetyl-L-methionine, N-acetyl-L-tyrosine, N-acetylserine, N-acetyl-D-glucosamine, N-acetyl-L-phenylalanine, N-acetylmannosamine, N-acetylneuraminic acid, N-acetylhistidine, N-acetylhydroxytryptamine, N-acetyltryptophan, N-acetylglutamine, N-acetyllysine, N-acetylornithine, N-methylnicotinamide, N-phenylacetyl-glutamine, N-phenylacetylphenylalanine, S-adenosine homocysteine, α-D-glucose, α-lactose, α-linolenic acid, α-hydroxyisobutyric acid, α-ketoglutaric acid, α-muricholic acid, β-D-trehalose, β-alanine, β-ursocholic acid, β-muricholic acid, γ-L-glutamyl-L-alanine, γ-linolenic acid, γ-aminobutyric acid, ω-muricholic acid, butyric acid, trimethylamine nitroxides, adenosine triphosphate, malonic acid, propionic acid, acetoacetic acid, acetylcysteine, acetylglycine, acetylornithine, acetic acid, guanidine acetate, glycolic acid, lactulose, lactoylglutathione, heneicosanoic acid, cis-12-heneicosenoic acid, heptacosanoic acid, tricosanoic acid, cis-14-tricosenoic acid, docosanoic acid, docosatrienoic acid, cis-13,16-docosadienoic acid, docosapentaenoic acid, docosahexaenoic acid, docosatetraenoic acid, trans-13-docosaenoic acid, cis-13-docosaenoic acid, pentacosanoic acid, octacosanoic acid, hexacosanoic acid, tetracosanoic acid, eicosanoic acid, eicosatrienoic acid, eicosadienoic acid, eicosapentaenoic acid, trans-II-eicosenoic acid, cis-ii-eicosenoic acid, cis-5-eicosenoic acid, cis-8-eicosenoic acid, dimethylglycine, adenosine diphosphate, linoleic acid, leucine, alloisoleucine, allolithocholic acid, allocholic acid, undecenoic acid, heptadecanoic acid, tridecanoic acid, nonadecanoic acid, nonadecadienoic acid, pentadecanoic acid, galactonic acid, galactitol, mecysteine, protocatechuic acid, apocholic acid, dehydrolithocholic acid, nordeoxycholic acid, trans-9-tetradecenoic acid, trans-aconitic acid, trans-4-hydroxyproline, trans-9-heptadecenoic acid, trans-9-pentadecenoic acid, trans-9-hexadecenoic acid, trans-linolenic acid, trans-cinnamic acid, elaidic acid, homocysteine, pyrrole-2-carboxylic acid, picolinic acid, indole, indole-3-methyl acetate, indole-3-carboxylic acid, indoleacetic acid, purine, azelaic acid, nonanoic acid, dopa, dopamine, melibiose, fumaric acid, acetaminophen, p-aminohippuric acid, p-cresol sulfate, symmetrical dimethylarginine, p-hydroxymandelic acid, p-hydroxyphenylacetic acid, homogentisic acid, adipic acid, pimelic acid, isobutyric acid, isoleucine, isovaleric acid, citric acid, isoursodeoxycholic acid, isohyodeoxycholic acid, isolithocholic acid, isocholic acid, isodeoxycholic acid, glutaric acid, glutaconic acid, valeric acid, mandelic acid, lauric acid, fructose-6-phosphate, citraconic acid, citric acid, citramalic acid, ribonolactone, ribonic acid, raffinose, palmitoleic acid, palmitic acid, norvaline, n-hydroxyphenylacetic acid, oxidized glutathione, aminoadipic acid, aminocaproic acid, salicyluric acid, oleic acid, trehalose, nicotinic acid, pyroglutamic acid, ursocholic acid, ursodeoxycholic acid, tauro-a-muricholic acid, tauro-b-muricholic acid, tauro-w-muricholic acid, tauroursodeoxycholic acid, taurohyocholic acid, taurohyodeoxycholic acid, taurolithocholic acid, taurocholic acid, taurodehydrocholic acid, taurochenodeoxycholic acid, hyocholic acid, hyodeoxycholic acid, succinylacetone, succinic acid, citrulline, glycodehydrocholic acid, glycolithocholic acid, glycodeoxycholic acid, glycine, glycochenodeoxycholic acid, glyceraldehyde, glyceraldehyde-3-phosphate, choline glycerophosphate, glycoursodeoxycholic acid, glycohyocholic acid, glycohyodeoxycholic acid, glycocholic acid, glyproline, glycyl-L-leucine, mannose-6-phosphoric acid, mannitol, methylmalonic acid, methylsuccinic acid, formic acid, capric acid, lithocholic acid, selenomethionine, thiamine, glycyllithocholic sulfate, stearic acid, liothyronine, dihydroxyacetone phosphate, phosphoribosyl pyrophosphate, creatine phosphate, hydroxypyruvic acid, glycine hydroxyphenylacetate, cinnamic acid, sarcosine, carnosine, creatine, inositol, epinephrine, choline, cholic acid, dehydrocholic acid, demethylcholic acid, adenosine monophosphate, arachidonic acid, phenylpyruvic acid, phenethylamine, phenylpropionic acid, phenyllactic acid, benzamide, benzoic acid, oxalic acid, shikimic acid, glucaric acid, glucose-6-phosphate, glucose lactone, sebacic acid, ricinoleic acid, sucrose, methionine sulfoxide, itaconic acid, melatonin, glutathione, myristic acid, erythronic acid, erythrose, suberic acid, caprylic acid, phthalic acid, tartaric acid, tyramine, quinic acid, m-aminobenzoic acid, asymmetric dimethylarginine, tannic acid, cis-10,12-octadecadienoic acid, cis-12,15-heneicosadienoic acid, cis-12-tridecenoic acid, cis-15-tetracosenic acid, cis-2-hydroxycinnamic acid, cis-9-tetradecenoic acid, cis-10-heptadecenoic acid, cis-11-dodecenoic acid, cis-4-hydroxyproline, cis-5-dodecenoic acid, cis-7-hexadecenoic acid, cis-9-heptadecenoic acid, cis-aconitic acid, vanillic acid, hippuric acid, maleic acid, homovanillic acid, ornithine, guanosine monophosphate, guanosine triphosphate, anserine, chenodeoxycholic acid, maltotriose, maltitol, rhamnose and murideoxycholic acid.

Specifically, the multiple metabolic components in the standard product and the quality control product may be selected from: fructose 1,6-diphosphate, 10Z-nonadecenoic acid, trans-11-octadecenoic acid, cis-11-octadecenoic acid, 12-dehydrocholic acid, 12-hydroxystearic acid, 12-ketolithocholic acid, 1-methylhistidine, 2,2-dimethylsuccinic acid, 2,3-diaminopropionic acid, glucoside 24-chenodeoxycholic acid, 2-butenoic acid, 2-oxoadipic acid, 2-methyl-4-pentenoic acid, 2-methyl-β-alanine, 2-methylbutyric acid, 2-methylhexanoic acid, 2-methylglutaric acid, 2-methylglutamic acid, 2-furoic acid, 2-hydroxy-2-methylbutyric acid, 2-hydroxy-3-methylbutyric acid, 2-hydroxybutyric acid, 2-hydroxycaproic acid, 2-hydroxycinnamic acid, 2-hydroxyphenylacetic acid, 2-hydroxyhippuric acid, 2-phenylpropionic acid, 2-phenylglycine, 3-(3-hydroxyphenyl)-3-hydroxypropionic acid, 3-(4-hydroxyphenyl)lactic acid, 3,4-dihydroxymandelic acid, 3,4-dihydroxyphenylpropionic acid, 3,4-dehydro-DL-proline, 3,5-diiodo-L-thyroxine, 30-cholic acid, 3-pyridineacetic acid, 3-indoleacrylic acid, 3-indolepropionic acid, 3-indoleacetamide, 3-oxyalanine, 3-aminosalicylic acid, 3-chloro-L-tyrosine, 3-methyl-2-oxobutyric acid, 3-methyl-2-oxovaleric acid, 3-methylindole, 3-methyladipic acid, 3-methylglutamic acid, 3-nitrotyrosine, sulfate 3-taurolithocholic acid, sulfate 3-lithocholic acid, 3-hydroxy-2-aminobenzoic acid, 3-hydroxybutyric acid, 3-hydroxypropionic acid, 3-hydroxyisovaleric acid, 3-hydroxyphenylacetic acid, 3-hydroxyhippuric acid, 3-dehydrocholic acid, 3-phenylbutyric acid, 4-methyl-2-oxovaleric acid, 4-methylhexanoic acid, 4-methoxyphenylacetic acid, 4-hydroxy-3-methoxymandelic acid, 4-hydroxycinnamic acid, 4-hydroxybenzoic acid, 4-hydroxyhippuric acid, 4-hydroxyphenyllactic acid, 4-hydroxyphenylpyruvic acid, 4-phenylbutyric acid, 5-aminolevulinic acid, 5-hydroxytryptophan, 5-hydroxytryptamine, 5-hydroxylysine, 6,7-diketolithocholic acid, 6-phosphogluconic acid, 6-ketolithocholic acid, 7,12-diketolithocholic acid, 7-ketolithocholic acid, 7-ketodeoxycholic acid, 9,11-conjugated linoleic acid, D-2-hydroxyglutaric acid, D-galactose, D-xylose, D-xylulose, D-fructose, D-ribose, D-ribose-5-phosphate, D-ribulose, D-ribulose-5-phosphate, D-mannose, D-glucose, D-maltose, L-2-aminobutyric acid, L-3-phenyllactic acid, L-alanine, L-serine, L-acetylcarnitine, L-lactic acid, L-allothreonine, L-cysteine, L-homoserine, L-homocysteine, L-homocitrulline, L-pipecolic acid, L-aspartic acid, L-asparagine, L-sorbose, L-lignic acid, L-norleucine, L-kynurenine, L-thyronine, L-arginine, L-histidine, L-valine, L-cystine, L-cystathionine, L-proline, L-tryptophan, L-threonine, L-phenylalanine, L-malic acid, L-methionine, L-glutamine, L-glutamic acid, L-lysine, L-tyrosine, L-arabinose, N-(3-phenylpropionyl)glycine, N-acetyl-L-alanine, N-acetyl-L-aspartic acid, N-acetyl-L-phenylalanine, N-acetyl-L-methionine, N-acetyl-L-tyrosine, N-acetylserine, N-acetyl-D-glucosamine, N-acetyl-L-phenylalanine, N-acetylmannosamine, N-acetylneuraminic acid, N-acetylhistidine, N-acetylhydroxytryptamine, N-acetyltryptophan, N-acetylglutamine, N-acetyllysine, N-acetylornithine, N-methylnicotinamide, N-phenylacetyl-glutamine, N-phenylacetylphenylalanine, S-adenosine homocysteine, α-D-glucose, α-lactose, α-linolenic acid, α-hydroxyisobutyric acid, α-ketoglutaric acid, α-muricholic acid, β-D-trehalose, β-alanine, β-ursocholic acid, β-muricholic acid, γ-L-glutamyl-L-alanine, γ-linolenic acid, γ-aminobutyric acid, ω-muricholic acid, butyric acid, trimethylamine nitroxides, adenosine triphosphate, malonic acid, propionic acid, acetoacetic acid, acetylcysteine, acetylglycine, acetylornithine, acetic acid, guanidine acetate, glycolic acid, lactulose, lactoylglutathione, heneicosanoic acid, cis-12-heneicosenoic acid, heptacosanoic acid, tricosanoic acid, cis-14-tricosenoic acid, docosanoic acid, docosatrienoic acid, cis-13,16-docosadienoic acid, docosapentaenoic acid, docosahexaenoic acid, docosatetraenoic acid, trans-13-docosaenoic acid, cis-13-docosaenoic acid, pentacosanoic acid, octacosanoic acid, hexacosanoic acid, tetracosanoic acid, eicosanoic acid, eicosatrienoic acid, eicosadienoic acid, eicosapentaenoic acid, trans-II-eicosenoic acid, cis-ii-eicosenoic acid, cis-5-eicosenoic acid, cis-8-eicosenoic acid, dimethylglycine, adenosine diphosphate, linoleic acid, leucine, alloisoleucine, allolithocholic acid, allocholic acid, undecenoic acid, heptadecanoic acid, tridecanoic acid, nonadecanoic acid, nonadecadienoic acid, pentadecanoic acid, galactonic acid, galactitol, mecysteine, protocatechuic acid, apocholic acid, dehydrolithocholic acid, nordeoxycholic acid, trans-9-tetradecenoic acid, trans-aconitic acid, trans-4-hydroxyproline, trans-9-heptadecenoic acid, trans-9-pentadecenoic acid, trans-9-hexadecenoic acid, trans-linolenic acid, trans-cinnamic acid, elaidic acid, homocysteine, pyrrole-2-carboxylic acid, picolinic acid, indole, indole-3-methyl acetate, indole-3-carboxylic acid, indoleacetic acid, purine, azelaic acid, nonanoic acid, dopa, dopamine, melibiose, fumaric acid, acetaminophen, p-aminohippuric acid, p-cresol sulfate, symmetrical dimethylarginine, p-hydroxymandelic acid, p-hydroxyphenylacetic acid, homogentisic acid, adipic acid, pimelic acid, isobutyric acid, isoleucine, isovaleric acid, citric acid, isoursodeoxycholic acid, isohyodeoxycholic acid, isolithocholic acid, isocholic acid, isodeoxycholic acid, glutaric acid, glutaconic acid, valeric acid, mandelic acid, lauric acid, fructose-6-phosphate, citraconic acid, citric acid, citramalic acid, ribonolactone, ribonic acid, raffinose, palmitoleic acid, palmitic acid, norvaline, n-hydroxyphenylacetic acid, oxidized glutathione, aminoadipic acid, aminocaproic acid, salicyluric acid, oleic acid, trehalose, nicotinic acid, pyroglutamic acid, ursocholic acid, ursodeoxycholic acid, tauro-a-muricholic acid, tauro-b-muricholic acid, tauro-w-muricholic acid, tauroursodeoxycholic acid, taurohyocholic acid, taurohyodeoxycholic acid, taurolithocholic acid, taurocholic acid, taurodehydrocholic acid, taurochenodeoxycholic acid, hyocholic acid, hyodeoxycholic acid, succinylacetone, succinic acid, citrulline, glycodehydrocholic acid, glycolithocholic acid, glycodeoxycholic acid, glycine, glycochenodeoxycholic acid, glyceraldehyde, glyceraldehyde-3-phosphate, choline glycerophosphate, glycoursodeoxycholic acid, glycohyocholic acid, glycohyodeoxycholic acid, glycocholic acid, glyproline, glycyl-L-leucine, mannose-6-phosphoric acid, mannitol, methylmalonic acid, methylsuccinic acid, formic acid, capric acid, lithocholic acid, selenomethionine, thiamine, glycyllithocholic sulfate, stearic acid, liothyronine, dihydroxyacetone phosphate, phosphoribosyl pyrophosphate, creatine phosphate, hydroxypyruvic acid, glycine hydroxyphenylacetate, cinnamic acid, sarcosine, carnosine, creatine, inositol, epinephrine, choline, cholic acid, dehydrocholic acid, demethylcholic acid, adenosine monophosphate, arachidonic acid, phenylpyruvic acid, phenethylamine, phenylpropionic acid, phenyllactic acid, benzamide, benzoic acid, oxalic acid, shikimic acid, glucaric acid, glucose-6-phosphate, glucose lactone, sebacic acid, ricinoleic acid, sucrose, methionine sulfoxide, itaconic acid, melatonin, glutathione, myristic acid, erythronic acid, erythrose, suberic acid, caprylic acid, phthalic acid, tartaric acid, tyramine, quinic acid, m-aminobenzoic acid, asymmetric dimethylarginine, tannic acid, cis-10,12-octadecadienoic acid, cis-12,15-heneicosadienoic acid, cis-12-tridecenoic acid, cis-15-tetracosenic acid, cis-2-hydroxycinnamic acid, cis-9-tetradecenoic acid, cis-10-heptadecenoic acid, cis-11-dodecenoic acid, cis-4-hydroxyproline, cis-5-dodecenoic acid, cis-7-hexadecenoic acid, cis-9-heptadecenoic acid, cis-aconitic acid, vanillic acid, hippuric acid, maleic acid, homovanillic acid, ornithine, guanosine monophosphate, guanosine triphosphate, anserine, chenodeoxycholic acid, maltotriose, maltitol, rhamnose and murideoxycholic acid.

In some embodiments, the number of the multiple metabolic components in the standard product and the quality control product is 50 or more, preferably 70 or more, further preferably 90 or more.

In some embodiments, the number of the multiple metabolic components in the standard product and the quality control product is 80 or more, preferably 90 or more, further preferably 100 or more.

In some embodiments, the number of the multiple metabolic components in the standard product and the quality control product is 200 or more, preferably 300 or more, further preferably 400 or more.

In some embodiments, the number of the multiple metabolic components in the standard product and the quality control product is 50 or more, preferably 100 or more, more preferably 200 or more, further preferably 600.

In some embodiments, the number of the multiple metabolic components in the standard product and the quality control product is 1000 or more.

A use method of the metabolic chip in the present invention includes the following steps:
1. collecting a biological sample;
2. according to the sample type, preparing a corresponding biological sample extract by using the corresponding method;
3. adding the prepared biological sample extract into each well of the metabolic chip in an equal amount, adding the same volume of a 3-nitrophenylhydrazine methanol solution and a 1-(3-dimethylaminopropyl)-3-ethylcarbodiimide pyridine solution into each well, and performing uniform vortex mixing and heating for derivatization, where the concentration of used 3-nitrophenylhydrazine is 100-320 nM, the concentration of 1-(3-dimethylaminopropyl)-3-ethylcarbodiimide is 50-200 nM, the reaction temperature is 20-60° C., and the reaction time is 10-120 minutes;
4. adding a carbon-13 labeled isotope internal standard solution obtained from the reaction of 3-nitrophenylhydrazine and 1-(3-dimethylaminopropyl)-3-ethylcarbodiimide into the derivatized biological sample extract obtained in step 3;
5. adding a methanol-water mixed solution into each well in the metabolic chip in step 4 for dilution, placing the metabolic chip in a tandem mass spectrometer for liquid chromatography-mass spectrometry for determination of amino acid, phenol, phenyl or benzyl derivative, indole, organic acid, fatty acid, sugar, and bile acid by liquid chromatography-mass spectrometry, and calculating concentrations of target metabolites in the sample based on results.

As understood by a person of ordinary skill in the art, the content of each target detection substance can be calculated based on detection results and the standard curve. The detection results obtained by the metabolic chip can also be obtained by calculation with a metabolite batch quantification software developed by Shenzhen Huiyun Biotechnology Co., Ltd. to quickly obtain the content of each target component. By combining the calculation software with the metabolic chip of the present invention, the work efficiency is greatly improved.

Traditional targeted metabolomics is constrained by predefined ion pair libraries, limiting its capacity for large-scale, precise metabolite quantification. This study proposes a revolutionary strategy—derivatization-AI synergy—to break the throughput bottleneck of metabolite quantification on triple quadrupole mass spectrometers for the first time. Core innovations include: (1) broad-spectrum functional group modification via derivatization reagents to significantly enhance ionization efficiency and generate predictable fragments; (2) GNN-based retention time prediction for high-confidence metabolite identification; and (3) TQMS-specific fragment ion scanning to improve quantitative specificity. The method overcomes the traditional coverage-precision conflict in metabolomics, enabling simultaneous quantification of >1,000 metabolites. AI-driven retention time calibration eliminates reliance on predefined spectral libraries, ensuring compatibility with any LC-MS platform and adaptability to diverse derivatization reagents. Validation in serum metabolome analysis identified novel low-abundance metabolite biomarkers linked to metabolic syndrome. This framework establishes a new paradigm for large-scale precision metabolomics.

### BRIEF DESCRIPTION OF THE DRAWINGS

The accompanying drawings are chromatograms of typical amino acid, organic acid, fatty acid, sugar, and bile acid detected in blood samples in embodiments. It can be seen that multiple target detection components can be effectively separated and detected, and thus high-throughput quantitative detection is achieved.

FIG. 1 is a chromatogram of typical short-chain fatty acid;

FIG. 2 is a chromatogram of typical amino acid;

FIG. 3 is a chromatogram of typical organic acid;

FIG. 4 is a chromatogram of typical sugar;

FIG. 5 is a chromatogram of typical bile acid.

### DETAILED DESCRIPTION OF THE INVENTION

### Example 1

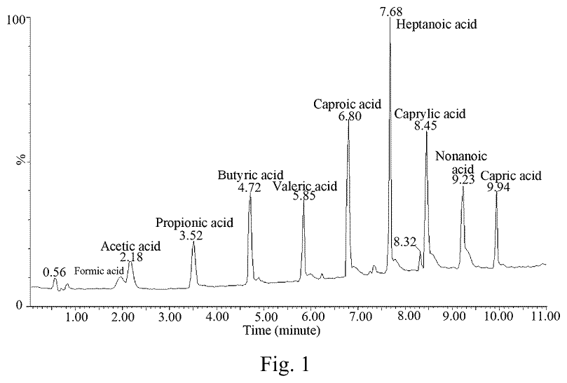

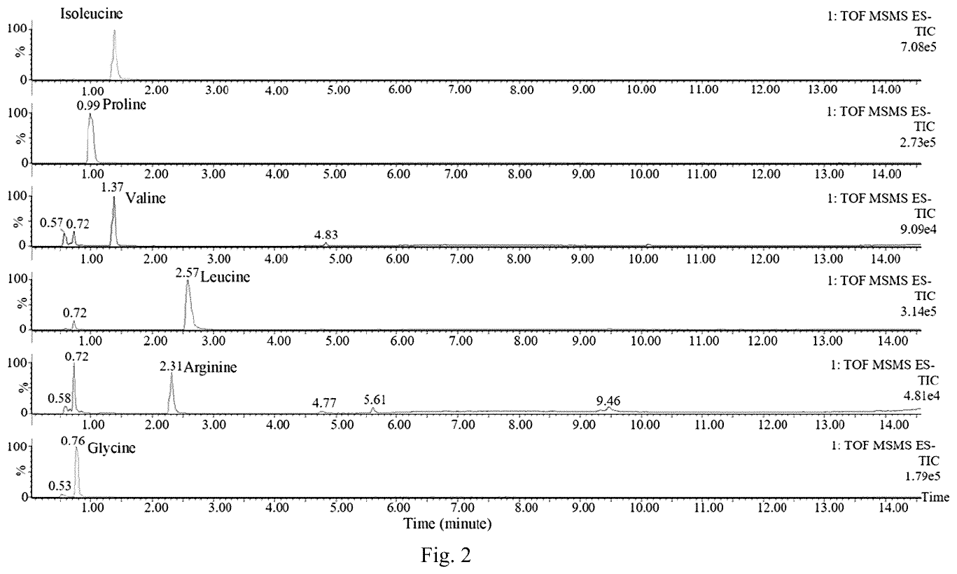

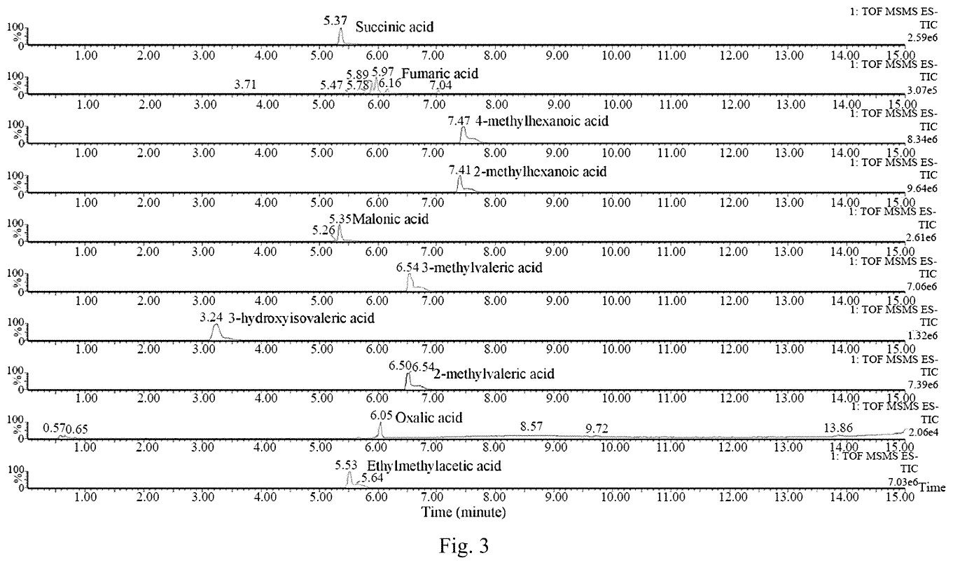

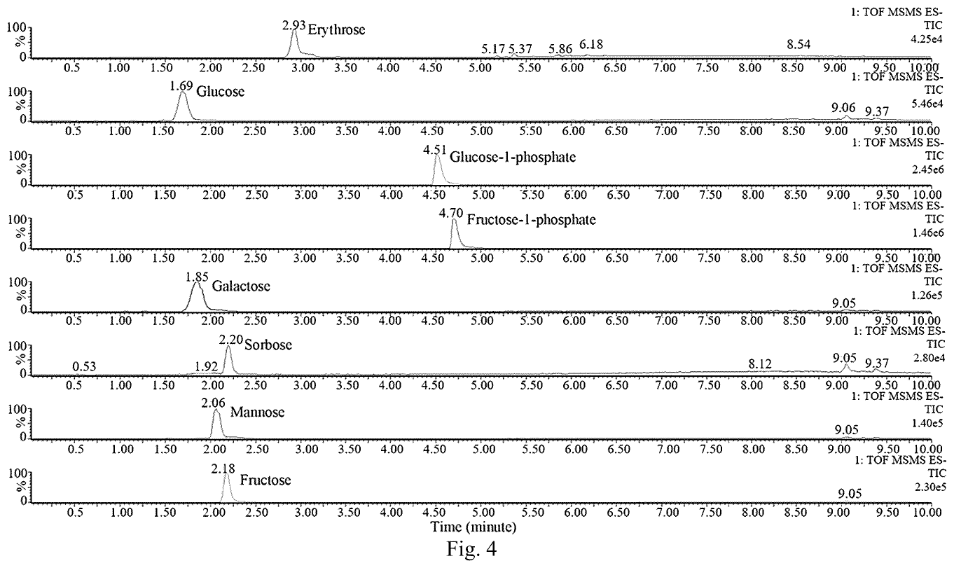

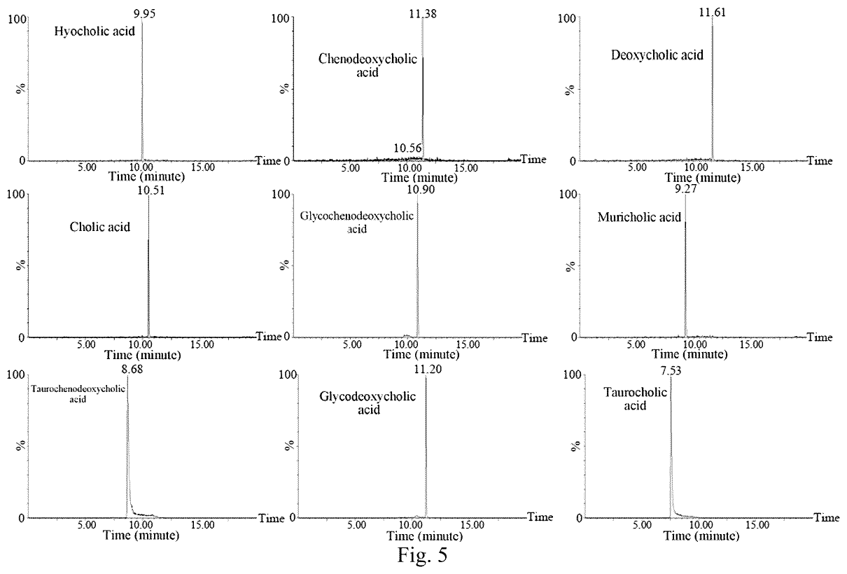

A metabolic chip is used to detect multiple index components in 10 human blood and fecal samples. FIGS. 1-5 are chromatograms of typical amino acid, organic acid, fatty acid, sugar, and bile acid detected in blood samples in this Example.

### 1. Instrument

Liquid chromatography-tandem mass spectrometer (LC-MS/MS) equipped with an electrospray ionization source (ESI).

### 2. Sample Preparation

Serum sample: Avenous whole blood sample is collected, placed in an anticoagulation tube, then immediately shaken up and down for uniform mixing 5-6 times, and centrifugated within 30 minutes to separate plasma. The sample is placed in a centrifuge tube, extracted with a mixed solvent of cold methanol, chloroform and water at a volume ratio of 3:1:1, shaken for a few seconds and then centrifugated at a rotation speed of 10000-20000 rpm at 4° C. for 5-15 minutes to obtain a supernatant. The supernatant is transferred into an autosampler glass vial. All water-containing serum or urine sample extracts are used for subsequent derivatization treatment.

Fecal sample: A fecal sample is freeze-dried. An appropriate amount of the freeze-dried fecal sample and an appropriate amount of a mixed solvent of cold methanol, chloroform, and water at a volume ratio of 3:1:1 are homogenized. The sample is centrifugated at a rotation speed of 10000-20000 rpm at 4° C. for 15-30 minutes to obtain a supernatant. The supernatant is transferred into an autosampler glass vial for subsequent derivatization treatment.

### 3. Reagent Preparation

Preparation of standard product solution: Standard products, including amino acid, phenol, phenyl or benzyl derivative, indole, organic acid, fatty acid, sugar, and bile acid, of metabolites in the description are taken, fully dissolved in methanol and uniformly mixed to prepare a 1 mg/ml solution, namely a concentrated stock solution which is prepared into a series of concentrations of solutions to draw a standard curve.

Preparation of quality control product solution: A corresponding quality control product is taken, fully dissolved in methanol and uniformly mixed to prepare a 1 mg/ml solution, namely a concentrated stock solution, and then the solution is diluted to a certain concentration and reacts with carbon-13 labeled 3-nitrophenylhydrazine and 1-(3-dimethylaminopropyl)-3-ethylcarbodiimide to obtain the quality control product solution.

Preparation of derivatization reagent (3-nitrophenylhydrazine): A derivatization reagent is uniformly mixed with a 75% methanol aqueous solution to prepare a 200 mM solution, and the solution is sealed and stored at 4° C. for later use.

Preparation of derivatization reaction catalyst (1-(3-dimethylaminopropyl)-3-ethylcarbodiimide): A reaction catalyst is prepared into a 120 millimolar solution with pyridine, and the solution is sealed and stored at 4° C. for later use.

### 4. Inspection Method

5 μL of a treated biological sample is taken, 20 μl of a derivatization reagent and 20 μl of a derivatization catalyst are added for reaction at 30° C. for 60 minutes, a methanol-water mixed solution is added into a reaction solution for dilution, centrifugation is performed at 13200 rpm for 15 minutes, and 5 μL of a supernatant is taken and introduced for LC-MS/MS analysis.

### Mass Spectrometry Conditions:

Ion source: Multi-reaction detection conditions for detection of multiple substances by tandem mass spectrometry are shown in Table 1. A negative ion scanning mode (ESI−) is adopted for an electrospray ion source, and specific conditions are as follows: The capillary voltage is 1.2 kV, the cone voltage is 55 V, the extraction cone voltage is 4 V, the ion source temperature is 150° C., the desolvent gas temperature is 550° C., the reverse cone gas flow is 50 L/h, the desolvent gas is 650 L/h, the resolution of a low mass zone is 4.7, the resolution of a high mass zone is 15, and a multi-reaction detection mode is used to collect data.

Gradient elution conditions: A UPLC BEH C18 chromatographic column (100 mm*2.1 mm, 1.7 μm) is used; the column temperature is 40° C.; a mobile phase A includes water (0.1% formic acid), and a mobile phase B includes acetonitrile (0.1% formic acid) and isopropanol at a ratio of (1-2):1; the flow rate is 0.4 mL/min; the injection volume is 5 microliters; and gradient elution conditions: 0-1 min (5% B), 1-5 min (5-30% B), 5-9 min (30-50% B), 9-12 min (50-75% B), 12-15 min (75-95% B), 15-16 min (95-100% B), 16-18 min (100% B), 18-20 min (5% B).

### 5. Determination Results

Concentrations of substances in 10 human blood and fecal samples are detected by using the detection method of the present invention (see Table 1 and Table 2 respectively). It can be seen that by using the method of the present invention to detect a single sample, multiple substances of different magnitudes and different properties can be quantitatively detected at one time.

TABLE 1
Determination results of concentrations
of substances in blood of normal people

| | Concentration | Concentration |
|---|---|---|
| Determined target metabolites | value | unit |
| 2-methylvaleric acid | 120.32 ± 5.18 | μg/mL |
| 2-hydroxybutyric acid | 2.32 ± 0.44 | μg/mL |
| 3-(3-hydroxyphenyl)-3- | 10.31 ± 0.19 | μg/mL |
| hydroxypropionic acid | | |
| 3-hydroxyphenylacetic acid | 6.07 ± 6.12 | μg/mL |
| 3-indoleacetonitrile | 2.51 ± 0.15 | μg/mL |
| 3-methyl-2-oxovaleric acid | 4.37 ± 5.08 | μg/mL |
| 4-methylhexanoic acid | 2.96 ± 0.07 | μg/mL |
| Linolenic acid | 97.19 ± 33.87 | μg/mL |
| Arachidonic acid | 5.54 ± 0.45 | μg/mL |
| Arachidonic acid | 5.62 ± 2.4 | μg/mL |
| Docosanoic acid | 8.96 ± 0.27 | μg/mL |
| β-alanine | 10.37 ± 0.44 | μg/mL |
| Capric acid | 1.22 ± 0.19 | μg/mL |
| Caprylic acid | 1.39 ± 0.5 | μg/mL |
| Citric acid | 7.03 ± 1.56 | μg/mL |
| Docosahexaenoic acid | 15.1 ± 4.36 | μg/mL |
| Docosapentaenoic acid n6 | 15.31 ± 4.16 | μg/mL |
| Docosatrienoic acid | 8.76 ± 0.95 | μg/mL |
| Dodecanoic acid | 0.91 ± 0.16 | μg/mL |
| Dopamine | 23.53 ± 3.93 | μg/mL |
| Eicosenoic acid | 11.18 ± 3.38 | μg/mL |
| Erucic acid | 15.25 ± 11.34 | μg/mL |
| γ-aminobutyric acid | 14.68 ± 2.7 | μg/mL |
| Glutathione | 6.7 ± 1.62 | μg/mL |
| Glycolic acid | 159.46 ± 56.76 | μg/mL |
| Heptadecanoic acid | 5.78 ± 6.5 | μg/mL |
| L-α-aminobutyric acid | 2.41 ± 0.48 | μg/mL |
| L-asparagine | 12.32 ± 0.57 | μg/mL |
| L-aspartic acid | 3.8 ± 0.64 | μg/mL |
| L-glutamic acid | 8.93 ± 0.53 | μg/mL |
| L-histidine | 16.76 ± 1.54 | μg/mL |
| L-homoserine | 12.53 ± 7.81 | μg/mL |
| L-isoleucine | 5.9 ± 2.18 | μg/mL |
| L-leucine | 5.88 ± 3.97 | μg/mL |
| L-lysine | 33.73 ± 4.42 | μg/mL |
| L-methionine | 6.78 ± 0.73 | μg/mL |
| L-norleucine | 3.33 ± 1.25 | μg/mL |
| L-phenylalanine | 8.57 ± 0.84 | μg/mL |
| L-proline | 11.32 ± 5.94 | μg/mL |
| L-serine | 9.78 ± 0.68 | μg/mL |
| L-tryptophan | 30.49 ± 2.93 | μg/mL |
| L-tyrosine | 20.02 ± 4.37 | μg/mL |
| L-valine | 28.87 ± 7.87 | μg/mL |
| Linoleic acid | 162.6 ± 57.21 | μg/mL |
| Methylsuccinic acid | 34.76 ± 29.31 | μg/mL |
| Myristic acid | 2.91 ± 0.52 | μg/mL |
| Myristic acid | 3.39 ± 0.51 | μg/mL |
| N-acetyltryptophan | 29.5 ± 1.24 | μg/mL |
| Nervonic acid | 82.71 ± 4.32 | μg/mL |
| Dodecanoic acid | 5.9 ± 2.13 | μg/mL |
| Norvaline | 0.86 ± 0.01 | μg/mL |
| Ornithine | 22.56 ± 5.54 | μg/mL |
| Carbonyladipic acid | 11.55 ± 2.52 | μg/mL |
| Oxoglutaric acid | 21.89 ± 5.22 | μg/mL |
| Palmitic acid | 87.29 ± 30.74 | μg/mL |
| Palmitoleic acid | 86.36 ± 30.14 | μg/mL |
| Pentadecanoic acid | 1.81 ± 0.12 | μg/mL |
| Pimelic acid | 4.47 ± 1.23 | μg/mL |
| Propionic acid | 0.16 ± 0.03 | μg/mL |
| Putrescine | 19.96 ± 1.32 | μg/mL |
| Pyroglutamic acid | 5.59 ± 2 | μg/mL |
| Stearic acid | 38.06 ± 14.04 | μg/mL |
| Succinic acid | 36.45 ± 12.11 | μg/mL |
| Cis-aconitic acid | 2.66 ± 0.11 | μg/mL |
| P-hydroxyphenylacetic acid | 2.51 ± 0.15 | μg/mL |
| Palmitoleic acid | 19.32 ± 11.42 | uM |
| Nervonic acid | 0.25 ± 0.06 | uM |
| Cholic acid | 67.57 ± 38.23 | nM |
| Chenodeoxycholic acid | 383 ± 559.28 | nM |
| Deoxycholic acid | 241.97 ± 197.98 | nM |
| Fructose | 5.23 ± 1.18 | uM |
| Glucose | 3.29 ± 0.66 | mM |
| Dohomo-g-linoleic acid | 2.51 ± 1.01 | uM |
| Mannose | 34.87 ± 9.22 | uM |
| Glycocholic acid | 314.89 ± 345.38 | nM |
| Glycochenodeoxycholic acid | 750.99 ± 574.2 | nM |
| Glycohyocholic acid | 16.31 ± 10.67 | nM |
| Glycolithocholic acid | 12.41 ± 10.41 | nM |
| Glycoursocholic acid | 123.97 ± 124.56 | nM |
| Hyocholic acid | 26.09 ± 11.26 | nM |
| Lithocholic acid | 12.61 ± 4.68 | nM |
| Oleic acid | 239.05 ± 84.26 | uM |
| Palmitoleic acid | 19.32 ± 11.42 | uM |
| Taurocholic acid | 88.59 ± 66.4 | nM |
| Taurodeoxycholic acid | 68.66 ± 47.85 | nM |
| Teracosanoic acid | 2.28 ± 0.89 | uM |
| Taurohyocholic acid | 4.06 ± 4.7 | nM |
| Tauroursocholic acid | 26.16 ± 0.81 | nM |
| Ursocholic acid | 69.29 ± 44.3 | nM |
| g-linoleic acid | 20.47 ± 8.36 | uM |

TABLE 2
Determination results of concentrations
of substances in feces of normal people

| | Concentration | Concentration |
|---|---|---|
| Detected target metabolites | value | unit |
| (1)-2-methylvaleric acid | 0.1 ± 0.19 | μg/mL |
| 1H-indole-3-acetamide | 0.54 ± 0.07 | μg/mL |
| 2-hydroxybutyric acid | 29.5 ± 7.89 | ng/mL |
| 3-(3-hydroxyphenyl)-3- | 0.19 ± 0.11 | μg/mL |
| hydroxypropionic acid | | |
| 3-hydroxybutyric acid | 0.3 ± 0.29 | μg/mL |
| 3-hydroxyphenylacetic acid | 0.39 ± 0.36 | μg/mL |
| 3-indoleacetonitrile | 0.17 ± 0.11 | μg/mL |
| 3-isopropionic acid | 0.32 ± 0.09 | μg/mL |
| 3-methyl-2-oxovaleric acid | 0.5 ± 0.2 | ng/mL |
| 3-methylvaleric acid | 0.2 ± 0.3 | ng/mL |
| 4-hydroxybenzoic acid | 0.42 ± 0.55 | μg/mL |
| 4-hydroxycinnamic acid | 0.25 ± 0.07 | μg/mL |
| 4-methylhexanoic acid | 0.7 ± 0.6 | ng/mL |
| 5-dodecenoic acid | 0.84 ± 2.1 | μg/mL |
| Adipic acid | 88.6 ± 10.6 | ng/mL |
| α-linolenic acid | 8.07 ± 9.95 | μg/mL |
| Aminoadipic acid | 0.14 ± 0.04 | μg/mL |
| Arachidonic acid | 3.83 ± 2.82 | μg/mL |
| Arachidonic acid | 0.97 ± 0.88 | μg/mL |
| Docosanoic acid | 0.33 ± 0.32 | μg/mL |
| β-alanine | 0.41 ± 0.12 | μg/mL |
| Butyric acid | 0.88 ± 0.69 | μg/mL |
| Capric acid | 0.13 ± 0.22 | μg/mL |
| Caproic acid | 0.53 ± 0.53 | μg/mL |
| Caprylic acid | 0.37 ± 0.55 | μg/mL |
| Citraconic acid | 45.8 ± 4.97 | ng/mL |
| 2-methylmalic acid | 0.18 ± 0.07 | μg/mL |
| Citric acid | 0.34 ± 0.32 | μg/mL |
| D-2-hydroxyglutaric acid | 0.14 ± 0.06 | μg/mL |
| Docosahexaenoic acid | 0.43 ± 0.3 | μg/mL |
| Docosapentaenoic acid n6 | 0.45 ± 0.41 | μg/mL |
| Docosatrienoic acid | 0.16 ± 0.02 | μg/mL |
| Dodecanoic acid | 1.07 ± 2.33 | μg/mL |
| Eicosenoic acid | 8.91 ± 6.15 | μg/mL |
| Erucic acid | 0.41 ± 0.36 | μg/mL |
| Ethylmethylacetic acid | 0.73 ± 0.38 | μg/mL |
| Fumaric acid | 0.12 ± 0.04 | μg/mL |
| γ-aminobutyric acid | 0.81 ± 0.37 | μg/mL |
| Glutaric acid | 0.32 ± 0.27 | μg/mL |
| Glutathione | 3.33 ± 5.08 | μg/mL |
| Glyceric acid | 4.11 ± 2.07 | μg/mL |
| Glycolic acid | 0.27 ± 0.21 | μg/mL |
| Heptadecanoic acid | 1.06 ± 1.15 | μg/mL |
| Heptanoic acid | 0.12 ± 0.15 | μg/mL |
| Homocysteine | l.l ± 1.01 | μg/mL |
| Hydrocinnamic acid | 0.36 ± 0.21 | μg/mL |
| Hydroxyphenyllactic acid | 0.48 ± 0.27 | μg/mL |
| Hydroxypropionic acid | 0.17 ± 0.2 | μg/mL |
| Indole | 0.4 ± 0.34 | μg/mL |
| Indoleacetic acid | 0.29 ± 0.02 | μg/mL |
| Isocitric acid | 89.5 ± 5.67 | ng/mL |
| Itaconic acid | 92.4 ± 15.4 | ng/mL |
| L-α-aminobutyric acid | 74.7 ± 16.7 | ng/mL |
| L-asparagine | 0.88 ± 0.59 | μg/mL |
| L-aspartic acid | 2.44 ± 1.03 | μg/mL |
| L-glutamic acid | 10.35 ± 8.3 | μg/mL |
| L-histidine | 0.39 ± 0.07 | μg/mL |
| L-homoserine | 0.45 ± 0.42 | μg/mL |
| L-isoleucine | 0.23 ± 0.11 | μg/mL |
| L-leucine | 0.25 ± 0.42 | μg/mL |
| L-lysine | 2.29 ± 0.65 | μg/mL |
| L-methionine | 1.54 ± 0.91 | μg/mL |
| L-norleucine | 0.13 ± 0.25 | μg/mL |
| L-phenylalanine | 0.85 ± 0.91 | μg/mL |
| L-proline | 0.4 ± 0.21 | μg/mL |
| L-serine | 0.42 ± 0.31 | μg/mL |
| L-tryptophan | 0.62 ± 0.16 | μg/mL |
| L-tyrosine | 2.19 ± 1.06 | μg/mL |
| L-valine | 1.05 ± 0.6 | μg/mL |
| Linoleic acid | 0.012 ± 0.05 | mg/mL |
| Malic acid | 0.38 ± 0.21 | μg/mL |
| Malonic acid | 0.12 ± 0.06 | μg/mL |
| Methylsuccinic acid | 0.13 ± 0.02 | μg/mL |
| Myristic acid | 0.54 ± 0.54 | μg/mL |
| Myristic acid | 0.53 ± 0.53 | μg/mL |
| N-acetyl | 0.61 ± 0.07 | μg/mL |
| Nervonic acid | 1.33 ± 0.9 | μg/mL |
| Nicotinic acid | 0.31 ± 0.09 | μg/mL |
| Dodecanoic acid | 0.21 ± 0.11 | μg/mL |
| Norvaline | 29.5 ± 4.56 | ng/mL |
| Ornithine | 1.29 ± 1.12 | μg/mL |
| Oxalic acid | 3.49 ± 5.07 | μg/mL |
| Carbonyladipic acid | 0.72 ± 0.41 | μg/mL |
| Oxoglutaric acid | 0.17 ± 0.17 | μg/mL |
| Palmitic acid | 3.79 ± 2.29 | μg/mL |
| Palmitoleic acid | 3.69 ± 2.19 | μg/mL |
| Nonanoic acid | 87.4 ± 12.5 | ng/mL |
| Pentadecanoic acid | 0.31 ± 0.24 | μg/mL |
| Phenol | 58.7 ± 6.78 | ng/mL |
| Phenylacetic acid | 1.49 ± 1.14 | μg/mL |
| Phenyllactic acid | 71.5 ± 8.79 | ng/mL |
| Pimelic acid | 0.31 ± 0.22 | μg/mL |
| Propionic acid | 10.09 ± 6.03 | μg/mL |
| Pyroglutamic acid | 2.6 ± 3.95 | μg/mL |
| Stearic acid | 1.22 ± 1.63 | μg/mL |
| Suberic acid | 0.11 ± 0.03 | μg/mL |
| Succinic acid | 0.42 ± 0.32 | μg/mL |
| Tartaric acid | 80.3 ± 9.97 | ng/mL |
| Thiamine | 2.23 ± 4.06 | μg/mL |
| Valeric acid | 1.39 ± 1.36 | μg/mL |
| Vanillic acid | 0.17 ± 0.04 | μg/mL |
| Cis-aconitic acid | 70.05 ± 10.08 | ng/mL |
| P-cresol | 0.24 ± 0.19 | μg/mL |
| P-hydroxyphenylacetic acid | 0.3 ± 0.22 | μg/mL |

### Example 2

A metabolic chip is used to detect multiple index components in 6 human blood and fecal samples.

### 1. Reagent Preparation

Preparation of standard product solution: Standard products, including amino acid, phenol, phenyl or benzyl derivative, indole, organic acid, fatty acid, sugar, and bile acid, of metabolites in the description are taken, fully dissolved in methanol and uniformly mixed to prepare a 1 mg/ml solution, namely a concentrated stock solution which is prepared into a series of concentrations of solutions to draw a standard curve.

Preparation of derivatization reagent (3-nitrophenylhydrazine): A derivatization reagent is uniformly mixed with a 75% methanol aqueous solution to prepare a 200 mM solution, and the solution is sealed and stored at 4° C. for later use.

Preparation of derivatization reaction catalyst (1-(3-dimethylaminopropyl)-3-ethylcarbodiimide): A reaction catalyst is prepared into a 120 millimolar solution with pyridine, and the solution is sealed and stored at 4° C. for later use.

### 2. Sample Preparation

Preparation of serum extraction: An aliquot of standard solution or 20 μL of serum was mixed with 120 μL of methanol. Centrifugation at 13,500 g and 4° C. for 10 min.

Preparation of feces extraction: Feces samples were freeze-dried, and approximately 5 mg of each sample was weighed. The samples were homogenized with 20 μL of ultrapure water and extracted with 120 μL of methanol. Centrifugation at 13,500 g and 4° C. for 10 min. L of a treated biological supernatant is taken, 20 μl of a derivatization reagent and 20 μl of a derivatization catalyst are added for reaction at 30° C. for 60 minutes, a methanol-water mixed solution is added into a reaction solution for dilution, centrifugation is performed at 13200 rpm for 15 minutes, and 5 μL of a supernatant is taken and introduced for LC-MS/MS analysis.

### 3. Inspection Method

### UHPLC/Q-Orbitrap-MS Analysis

High-resolution mass spectrometry (HRMS) data for multicomponent characterization and untargeted metabolomics were acquired using a Vanquish Flex UHPLC system coupled to a Q Extractive Focus Orbitrap mass spectrometer equipped with a heated electrospray ionization (ESI) source (Thermo Fisher Scientific, San Jose, CA, USA). Chromatographic separation was performed on a reversed-phase ACQUITY UPLC BEH C18 column (2.1 mm×100 mm, 1.7 m particle size) using a binary gradient elution with mobile phases: 0.1% formic acid in water (solvent A) and acetonitrile containing 0.1% formic acid/isopropanol (70:30, v/v, solvent B). The column temperature is 40° C. The flow rate is 0.4 mL/min; the injection volume is 5 microliters. The gradient program was as follows: 0-1 min (5% B), 1-5 min (5-30% B), 5-9 min (30-50% B), 9-12 min (50-75% B), 12-15 min (75-95% B), 15-16 min (95-100% B), 16-18 min (100% B), 18-20 min (5% B).

Mass Spectrometry Parameters: The mass spectrometry scan parameters were set as follows: S-lens RF level, 50; mass range, 100 to 1200 m/z; full MS resolution, 70,000; MS/MS resolution, 17,500; NCE/stepped NCE was set at 10, 20, and 40 eV. The ESI ion source parameters were set as follows: spray voltage, 3800 V; sheath gas flow rate, 40 for ESI+ and 5 for ESI−; capillary temperature, 320° C.; probe heater temperature, 350° C.; aux gas flow rate, 10.

### 4. Determination Results

Concentrations of substances in 6 human serum and fecal samples are detected by using the detection method of the present invention (Table 3). It can be seen that by using the method of the present invention to detect a single sample, multiple substances of different magnitudes and different properties can be quantitatively detected at one time.

TABLE 3
Determination results of concentrations of substances in blood and feces of normal people

| | | | RT | Reference | Fecal | Serum |
|---|---|---|---|---|---|---|
| NO. | Name | m/z | (min) | Ion | (Intensity) | (Intensity) |
| 1 | Propynoic acid | 264.06 | 6.246 | [M − H + HAc] − 1 | 2510269 ± | 841952 ± |
| | | | | | 92554 | 28455 |
| 2 | Acrylic acid | 208.07 | 0.883 | [M + H] + 1 | 13865154 ± | 14228426 ± |
| | | | | | 12993928 | 23777442 |
| 3 | Glyoxylic acid | 208.04 | 5.832 | [M − H] − 1 | 12000471 ± | 2144384 ± |
| | | | | | 2000381 | 228813 |
| 4 | Isocrotonic acid | 239.11 | 0.434 | [M + NH4] + 1 | 266059225 ± | 218886 ± |
| | | | | | 460019188 | 43040 |
| 5 | 2-Aminoacrylic acid | 221.07 | 6.247 | [M − H] − 1 | 3464018 ± | 6160226 ± |
| | | | | | 800230 | 1903288 |
| 6 | Malonic semialdehyde | 222.05 | 9.361 | [M − H] − 1 | 8953281 ± | 1962295 ± |
| | | | | | 3833020 | 1202549 |
| 7 | 2-hydroxyacrylic Acid | 222.05 | 7.364 | [M − H] − 1 | 40949997 ± | 9472056 ± |
| | | | | | 4052812 | 1132094 |
| 8 | (alpha-D-mannosyl)7-beta-D-mannosyl- | 223.08 | 1.516 | [M − H] − 1 | 458487665 ± | 148636884 ± |
| | diacetylchitobiosyl-L-asparagine, | | | | 63646212 | 13164304 |
| | isoform A (protein) | | | | | |
| 9 | Monoethyl carbonate | 224.07 | 4.177 | [M − H] − 1 | 246941092 ± | 71459981 ± |
| | | | | | 7837139 | 3414768 |
| 10 | Methoxyacetic acid | 224.07 | 3.726 | [M − H] − 1 | 5184592776 ± | 2172177007 ± |
| | | | | | 72558533 | 122437118 |
| 11 | 2-hydroxypropanoic acid | 224.07 | 5.824 | [M − H] − 1 | 157271490 ± | 8346423 ± |
| | | | | | 13475327 | 299209 |
| 12 | Senecioic acid | 236.10 | 2.004 | [M + H] + 1 | 76683919 ± | 10848175 ± |
| | | | | | 56264980 | 10483810 |
| 13 | Tiglic acid | 253.13 | 0.6 | [M + NH4] + 1 | 1509117642 ± | 58052130 ± |
| | | | | | 2611743446 | 35789141 |
| 14 | 2-Ethylacrylic acid | 253.13 | 1.254 | [M + NH4] + 1 | 1862909805 ± | 2059982 ± |
| | | | | | 1650227504 | 695343 |
| 15 | Angelic acid | 253.13 | 1.784 | [M + NH4] + 1 | 50362708 ± | 9238657 ± |
| | | | | | 86502238 | 4771737 |
| 16 | 2-Ketobutyric acid | 236.07 | 7.196 | [M − H] − 1 | 1787233 ± | 3482001 ± |
| | | | | | 91095 | 1733281 |
| 17 | Succinic acid semialdehyde | 236.07 | 4.368 | [M − H] − 1 | 16202483 ± | 4515125 ± |
| | | | | | 4170048 | 168205 |
| 18 | 4-Hydroxycrotonic acid | 236.07 | 8.057 | [M − H] − 1 | 142453769 ± | 31505827 ± |
| | | | | | 11877298 | 3548011 |
| 19 | (S)-2-Methylbutanoic acid | 236.10 | 6.982 | [M − H] − 1 | 3463810327 ± | 7900237 ± |
| | | | | | 108796706 | 1585336 |
| 20 | Pivalic acid | 236.10 | 7.443 | [M − H] − 1 | 6972066992 ± | 8738812 ± |
| | | | | | 105310392 | 1653469 |
| 21 | Pyruvatoxime | 237.06 | 2.504 | [M − H] − 1 | 5796134 ± | 248976 ± |
| | | | | | 360009 | 125681 |
| 22 | (R)-beta-Aminoisobutyric acid | 237.10 | 3.625 | [M − H] − 1 | 378160 ± | 176939 ± |
| | | | | | 31006 | 18988 |
| 23 | N-Ethylglycine | 237.10 | 2.174 | [M − H] − 1 | 12053298 ± | 812656 ± |
| | | | | | 1665099 | 97973 |
| 24 | N-Methylalanine | 237.10 | 2.6 | [M − H] − 1 | 7789054 ± | 2567964 ± |
| | | | | | 1095566 | 546229 |
| 25 | L-alpha-Aminobutyric acid | 237.10 | 1.67 | [M − H] − 1 | 49720914 ± | 3302799 ± |
| | | | | | 41479142 | 417895 |
| 26 | Tartronate semialdehyde | 238.05 | 4.388 | [M − H] − 1 | 3874452 ± | 1242695 ± |
| | | | | | 286591 | 81426 |
| 27 | 3-Hydroxybutyric acid | 238.08 | 4.952 | [M − H] − 1 | 301806216 ± | 103894796 ± |
| | | | | | 5243093 | 15933411 |
| 28 | (S)-3-Hydroxyisobutyric acid | 238.08 | 4.035 | [M − H] − 1 | 102000710 ± | 198892671 ± |
| | | | | | 2677646 | 6454186 |
| 29 | (R)-3-Hydroxyisobutyric acid | 238.08 | 4.434 | [M − H] − 1 | 5691133 ± | 8508157 ± |
| | | | | | 4272373 | 1139426 |
| 30 | 4-Carboxypyrazole | 306.08 | 2.842 | [M − H + HAc] − 1 | 4643029 ± | 54817 ± |
| | | | | | 487793 | 2815 |
| 31 | (2E,4E)-2,4-Hexadienoic acid | 248.10 | 1.793 | [M + H] + 1 | 440676 ± | 353192 ± |
| | | | | | 407639 | 230407 |
| 32 | 1-Pyrroline-2-carboxylic acid | 247.08 | 5.843 | [M − H] − 1 | 510503052 ± | 726932 ± |
| | | | | | 6210566 | 150549 |
| 33 | cis-Acetylacrylate | 248.07 | 7.356 | [M − H] − 1 | 474385 ± | 15651520 ± |
| | | | | | 176040 | 2408852 |
| 34 | L-3-Cyanoalanine | 248.07 | 9.49 | [M − H] − 1 | 1059622 ± | 5370099 ± |
| | | | | | 22694 | 434557 |
| 35 | 4-Amino-2-methylenebutanoic acid | 249.10 | 2.188 | [M − H] − 1 | 87350177 ± | 56666811 ± |
| | | | | | 13490729 | 6935788 |
| 36 | Levulinic acid | 250.08 | 6.464 | [M − H] − 1 | 178720991 ± | 176955 ± |
| | | | | | 147448038 | 11387 |
| 37 | 2-Oxovaleric acid | 250.08 | 9.01 | [M − H] − 1 | 9445509 ± | 945025 ± |
| | | | | | 2098125 | 61720 |
| 38 | 2-Methylacetoacetic acid | 250.08 | 7.484 | [M − H] − 1 | 7951379 ± | 8911936 ± |
| | | | | | 880664 | 4062180 |
| 39 | 2-Ethylbutanoic acid | 250.12 | 9.023 | [M − H] − 1 | 12672521 ± | 4951315 ± |
| | | | | | 4077090 | 786439 |
| 40 | L-2-Amino-3-oxobutanoic acid | 253.09 | 1.836 | [M + H] + 1 | 73838201 ± | 320289 ± |
| | | | | | 63551844 | 140244 |
| 41 | L-Aspartate-semialdehyde | 251.08 | 3.026 | [M − H] − 1 | 436917309 ± | 4211220 ± |
| | | | | | 36351677 | 762056 |
| 42 | N-Methyl-a-aminoisobutyric acid | 251.11 | 1.92 | [M − H] − 1 | 121887846 ± | 61259 ± |
| | | | | | 11507581 | 3837 |
| 43 | 5-Aminopentanoic acid | 253.13 | 0.668 | [M + H] + 1 | 3918736 ± | 25332320 ± |
| | | | | | 6415609 | 20914439 |
| 44 | 4-Hydroxy-2-oxobutanoic acid | 252.06 | 4.852 | [M − H] − 1 | 27476587 ± | 343028 ± |
| | | | | | 1089113 | 141601 |
| 45 | xi-3-Hydroxy-2-oxobutanoic acid | 312.08 | 3.66 | [M − H + HAc] − 1 | 29764174 ± | 77247104 ± |
| | | | | | 6501749 | 9010510 |
| 46 | 3-methoxy-3-oxopropanoic acid | 312.08 | 3.087 | [M − H + HAc] − 1 | 10120248 ± | 51191 ± |
| | | | | | 2417413 | 3026 |
| 47 | Fatty acids and conjugates | 254.11 | 2.641 | [M + H] + 1 | 31771483 ± | 215413 ± |
| | | | | | 19499829 | 173051 |
| 48 | Fatty acids and conjugates | 254.11 | 2.596 | [M + H] + 1 | 34971729 ± | 974769 ± |
| | | | | | 59722626 | 785010 |
| 49 | Fatty acids and conjugates | 252.10 | 6.113 | [M − H] − 1 | 2254633676 ± | 72759156 ± |
| | | | | | 58768592 | 8909091 |
| 50 | Fatty acids and conjugates | 252.10 | 4.507 | [M − H] − 1 | 358893190 ± | 618874 ± |
| | | | | | 5309460 | 77632 |
| 51 | Fatty acids and conjugates | 252.10 | 5.158 | [M − H] − 1 | 14144656 ± | 3494450 ± |
| | | | | | 3243962 | 686593 |
| 52 | Fatty acids and conjugates | 252.10 | 4.92 | [M − H] − 1 | 49093638 ± | 7131098 ± |
| | | | | | 4340581 | 2311977 |
| 53 | L-Allothreonine | 253.09 | 1.592 | [M − H] − 1 | 23010815 ± | 3858495 ± |
| | | | | | 3438091 | 570302 |
| 54 | 3-Methylthiopropionic acid | 254.06 | 6.098 | [M − H] − 1 | 167272734 ± | 122956 ± |
| | | | | | 7300773 | 12506 |
| 55 | (S)-3,4-Dihydroxybutyric acid | 254.08 | 2.666 | [M − H] − 1 | 10373529 ± | 2146916 ± |
| | | | | | 1019102 | 252088 |
| 56 | 2,4-Dihydroxybutanoic acid | 254.08 | 3.154 | [M − H] − 1 | 14557796 ± | 1523900 ± |
| | | | | | 2164021 | 145535 |
| 57 | 4-Deoxyerythronic acid | 254.08 | 2.641 | [M − H] − 1 | 10369551 ± | 2127538 ± |
| | | | | | 972695 | 272618 |
| 58 | 4-Deoxythreonic acid | 254.08 | 2.92 | [M − H] − 1 | 809070 ± | 3564964 ± |
| | | | | | 292135 | 438578 |
| 59 | A,b-Dihydroxyisobutyric acid | 254.08 | 5.21 | [M − H] − 1 | 23965796 ± | 10407027 ± |
| | | | | | 543134 | 2942333 |
| 60 | Isonicotinic acid | 259.08 | 2.651 | [M + H] + 1 | 102305718 ± | 105725 ± |
| | | | | | 82584803 | 114332 |
| 61 | 5-Methylfuran-2-carboxylic acid | 262.08 | 2.396 | [M + H] + 1 | 188653836 ± | 8356972 ± |
| | | | | | 132888236 | 9881604 |
| 62 | Imidazoleacetic acid | 262.08 | 1.284 | [M + H] + 1 | 2506979 ± | 69477 ± |
| | | | | | 1328936 | 14881 |
| 63 | 2-Heptenoic acid | 262.12 | 8.472 | [M − H] − 1 | 1240023698 ± | 284926 ± |
| | | | | | 112948294 | 39986 |
| 64 | 1-Pyrroline-4-hydroxy-2-carboxylate | 282.12 | 1.05 | [M + NH4] + 1 | 33383990 ± | 392110 ± |
| | | | | | 33487341 | 450883 |
| 65 | Pyrroline hydroxycarboxylic acid | 265.09 | 2.251 | [M + H] + 1 | 384364073 ± | 29327068 ± |
| | | | | | 43796349 | 48411341 |
| 66 | (3R,5S)-1-pyrroline-3-hydroxy-5- | 263.08 | 3.694 | [M − H] − 1 | 3855850257 ± | 714755282 ± |
| | carboxylic Acid | | | | 223920707 | 102960754 |
| 67 | Vigabatrin | 265.13 | 1.761 | [M + H] + 1 | 37085635 ± | 38956215 ± |
| | | | | | 29307615 | 33732957 |
| 68 | Methyl hydrogen fumarate | 264.06 | 4.297 | [M − H] − 1 | 94878164 ± | 1018471 ± |
| | | | | | 6244568 | 65304 |
| 69 | 2-Hydroxyglutaric acid lactone | 264.10 | 11.6 | [M − H] − 1 | 25655400 ± | 22813723 ± |
| | | | | | 17304663 | 6117694 |
| 70 | Adipate semialdehyde | 264.10 | 10.037 | [M − H] − 1 | 20240698 ± | 4271382 ± |
| | | | | | 888382 | 220403 |
| 71 | 4-Acetylbutyrate | 264.10 | 8.366 | [M − H] − 1 | 1595053 ± | 3000545 ± |
| | | | | | 102771 | 823663 |
| 72 | 2-Methyl-3-ketovaleric acid | 264.10 | 7.226 | [M − H] − 1 | 2291298 ± | 1838653 ± |
| | | | | | 171975 | 443476 |
| 73 | 2-Ketohexanoic acid | 264.14 | 9.335 | [M − H] − 1 | 1911246 ± | 328137 ± |
| | | | | | 82034 | 170919 |
| 74 | 3-Oxohexanoic acid | 264.14 | 9.786 | [M − H] − 1 | 11778657 ± | 6661033 ± |
| | | | | | 265936 | 1148748 |
| 75 | 2,5-Dioxopentanoate | 264.14 | 9.499 | [M − H] − 1 | 2791835 ± | 590708 ± |
| | | | | | 847837 | 306700 |
| 76 | L-Glutamic gamma-semialdehyde | 265.09 | 3.779 | [M − H] − 1 | 1534024418 ± | 2205185 ± |
| | | | | | 48867745 | 1409406 |
| 77 | 3-Hydroxy-L-proline | 265.09 | 1.517 | [M − H] − 1 | 5432848 ± | 14927015 ± |
| | | | | | 611545 | 2122487 |
| 78 | 5-Amino-2-oxopentanoic acid | 265.09 | 4.176 | [M − H] − 1 | 593323230 ± | 361362 ± |
| | | | | | 14981948 | 32619 |
| 79 | Beta-Guanidinopropionic acid | 267.12 | 0.553 | [M + H] + 1 | 35379585 ± | 10444853 ± |
| | | | | | 58571246 | 7341921 |
| 80 | N-methylvaline | 265.13 | 4.995 | [M − H] − 1 | 224420851 ± | 58040577 ± |
| | | | | | 18191555 | 9624895 |
| 81 | L-Alloisoleucine | 265.13 | 4.809 | [M − H] − 1 | 29819470 ± | 3491846 ± |
| | | | | | 542615 | 741341 |
| 82 | Beta-Leucine | 325.15 | 1.786 | [M − H + HAc] − 1 | 16061225 ± | 223094 ± |
| | | | | | 2695564 | 69164 |
| 83 | (S)-Methylbutanethioic acid | 268.09 | 2.947 | [M + H] + 1 | 78649134 ± | 50836 ± |
| | | | | | 12298198 | 18687 |
| 84 | 2-Hydroxy-4-oxopentanoic acid | 268.09 | 3.054 | [M + H] + 1 | 12626453 ± | 12529 ± |
| | | | | | 1716166 | 469 |
| 85 | 2-Acetolactate | 266.08 | 5.355 | [M − H] − 1 | 498744073 ± | 1437981 ± |
| | | | | | 31880606 | 311915 |
| 86 | N-Carbamoylsarcosine | 266.11 | 7.145 | [M − H] − 1 | 6865075012 ± | 3705564 ± |
| | | | | | 289785550 | 2438069 |
| 87 | 5-Hydroxyhexanoic acid | 266.11 | 5.321 | [M − H] − 1 | 16277930 ± | 262350 ± |
| | | | | | 290543 | 129908 |
| 88 | 2-Ethyl-2-Hydroxybutyric acid | 266.11 | 7.407 | [M − H] − 1 | 272231157 ± | 546354 ± |
| | | | | | 18134695 | 366058 |
| 89 | L-2-Amino-5-hydroxypentanoic acid | 267.11 | 1.416 | [M − H] − 1 | 1698430 ± | 90915 ± |
| | | | | | 352622 | 8586 |
| 90 | 2,3-Dihydroxyvaleric acid | 268.09 | 5.419 | [M − H] − 1 | 1061686411 ± | 104712 ± |
| | | | | | 20065806 | 24588 |
| 91 | 2-Methylbenzoic acid | 270.09 | 7.638 | [M − H] − 1 | 2676986841 ± | 10257108 ± |
| | | | | | 122859059 | 1274107 |
| 92 | M-toluic Acid | 270.09 | 10.502 | [M − H] − 1 | 8083724 ± | 5066654 ± |
| | | | | | 500455 | 742473 |
| 93 | 2-Pyridylacetic acid | 271.08 | 6.732 | [M − H] − 1 | 515650 ± | 54619 ± |
| | | | | | 130925 | 36348 |
| 94 | 2-Aminonicotinic acid | 274.09 | 1.901 | [M + H] + 1 | 353328922 ± | 2232782 ± |
| | | | | | 301069464 | 1384730 |
| 95 | Trans-urocanate | 274.09 | 1.327 | [M + H] + 1 | 97073030 ± | 1579413 ± |
| | | | | | 166209235 | 861675 |
| 96 | Methylimidazoleacetic acid | 276.11 | 1.208 | [M + H] + 1 | 299197480 ± | 10759423 ± |
| | | | | | 289848389 | 961891 |
| 97 | Pi-Methylimidazoleacetic acid | 276.11 | 0.581 | [M + H] + 1 | 165875087 ± | 1786900 ± |
| | | | | | 286015596 | 899069 |
| 98 | 2-Propyl-2,4-pentadienoic acid | 274.09 | 1.884 | [M − H] − 1 | 115357069 ± | 508197 ± |
| | | | | | 12378801 | 352127 |
| 99 | Arecaidine | 275.11 | 8.359 | [M − H] − 1 | 95267782 ± | 24186 ± |
| | | | | | 6940621 | 626 |
| 100 | L-Hypoglycin A | 335.14 | 8.481 | [M − H + HAc] − 1 | 10331411 ± | 32943 ± |
| | | | | | 597912 | 1634 |
| 101 | 2-Octenoic acid | 276.14 | 9.801 | [M − H] − 1 | 9276052 ± | 4416796 ± |
| | | | | | 374158 | 736373 |
| 102 | 6-Oxopiperidine-2-carboxylic acid | 277.09 | 6.043 | [M − H] − 1 | 38049394 ± | 367162 ± |
| | | | | | 1546962 | 53838 |
| 103 | L-2-Amino-3-methylenehexanoic acid | 279.14 | 2.665 | [M + H] + 1 | 10544073 ± | 630369 ± |
| | | | | | 2839726 | 1006667 |
| 104 | 3-Hydroxyadipic acid 3,6-lactone | 297.12 | 2.587 | [M + NH4] + 1 | 21236735 ± | 78280 ± |
| | | | | | 21931240 | 103829 |
| 105 | 4-Hydroxycyclohexylcarboxylic acid | 278.11 | 4.88 | [M − H] − 1 | 157503606 ± | 183149 ± |
| | | | | | 4691297 | 40988 |
| 106 | 2-Methylheptanoic acid | 278.15 | 10.759 | [M − H] − 1 | 20338229 ± | 20473965 ± |
| | | | | | 1864306 | 2435501 |
| 107 | Allysine | 279.11 | 1.108 | [M − H] − 1 | 2807182 ± | 314493 ± |
| | | | | | 696717 | 20222 |
| 108 | L-trans-5-Hydroxy-2- | 279.11 | 3.341 | [M − H] − 1 | 1423756 ± | 37975 ± |
| | piperidinecarboxylic acid | | | | 268610 | 2412 |
| 109 | N-(2-Carboxymethyl)-morpholine | 279.11 | 4.728 | [M − H] − 1 | 1095546044 ± | 407933 ± |
| | | | | | 71038961 | 163145 |
| 110 | N-Propionylalanine | 279.11 | 5.296 | [M − H] − 1 | 497524956 ± | 48065 ± |
| | | | | | 29571905 | 8053 |
| 111 | 4-Guanidinobutanoic acid | 279.12 | 2.486 | [M − H] − 1 | 1581679 ± | 88408 ± |
| | | | | | 213311 | 5947 |
| 112 | 2-Aminoheptanoate | 281.16 | 1.415 | [M + H] + 1 | 27567023 ± | 178365 ± |
| | | | | | 23590026 | 42513 |
| 113 | Monomethyl glutaric acid | 282.11 | 3.182 | [M + H] + 1 | 26040569 ± | 389393 ± |
| | | | | | 6174601 | 14572 |
| 114 | (3S)-3,6-Diaminohexanoate | 280.14 | 0.785 | [M − H] − 1 | 14874828 ± | 1136438 ± |
| | | | | | 3770143 | 221340 |
| 115 | (3S,5S)-3,5-Diaminohexanoate | 282.16 | 0.271 | [M + H] + 1 | 3320125 ± | 17033 ± |
| | | | | | 5722971 | 943 |
| 116 | L-4-Hydroxyglutamate semialdehyde | 283.10 | 1.575 | [M + H] + 1 | 87944654 ± | 8731200 ± |
| | | | | | 75853946 | 12166455 |
| 117 | O-Acetylserine | 283.10 | 1.075 | [M + H] + 1 | 157295107 ± | 14428675 ± |
| | | | | | 269629227 | 11991693 |
| 118 | (2R,3R,4R)-2-Amino-4-hydroxy-3- | 341.15 | 1.511 | [M − H + HAc] − 1 | 2434963 ± | 12879539 ± |
| | methylpentanoic acid | | | | 635237 | 3090466 |
| 119 | 3-phenylprop-2-enoic acid | 284.10 | 3.829 | [M + H] + 1 | 356811 ± | 69236 ± |
| | | | | | 97593 | 13797 |
| 120 | trans-Cinnamic acid | 282.09 | 8.72 | [M − H] − 1 | 674959 ± | 229561 ± |
| | | | | | 103340 | 287646 |
| 121 | Penicillamine | 283.09 | 4.195 | [M − H] − 1 | 150845352 ± | 7917189 ± |
| | | | | | 705082 | 696903 |
| 122 | (\xc2\xb1)-2-Hydroxy-4- | 284.07 | 6.022 | [M − H] − 1 | 251478688 ± | 101178 ± |
| | (methylthio)butanoic acid | | | | 25531414 | 33234 |
| 123 | alpha-Fluoro-beta-ureidopropionic acid | 286.10 | 2.635 | [M + H] + 1 | 114904443 ± | 41101 ± |
| | | | | | 68266446 | 24897 |
| 124 | 2-Deoxypentonic acid | 286.10 | 2.664 | [M + H] + 1 | 43720321 ± | 112407 ± |
| | | | | | 29766910 | 13529 |
| 125 | 2-Deoxyribonic acid | 286.10 | 2.589 | [M + H] + 1 | 63405399 ± | 19779 ± |
| | | | | | 88251992 | 4806 |
| 126 | 3-hydroxy-2,2- | 284.09 | 2.71 | [M − H] − 1 | 14187916 ± | 298762 ± |
| | bis(hydroxymethyl)propanoic acid | | | | 2142313 | 101073 |
| 127 | 3,4-Dimethylbenzoic acid | 284.10 | 8.475 | [M − H] − 1 | 1373693518 ± | 4377638 ± |
| | | | | | 133937468 | 830103 |
| 128 | 4-Ethylbenzoic acid | 285.10 | 4.733 | [M − H] − 1 | 391105 ± | 61527 ± |
| | | | | | 6989 | 46564 |
| 129 | 3-Cresotinic acid | 286.08 | 6.152 | [M − H] − 1 | 76223888 ± | 214577 ± |
| | | | | | 2500612 | 139919 |
| 130 | 4-Hydroxy-3-methylbenzoic acid | 286.08 | 5.692 | [M − H] − 1 | 117011496 ± | 1211709 ± |
| | | | | | 1446748 | 282448 |
| 131 | 2-Methoxybenzoic acid | 286.08 | 6.66 | [M − H] − 1 | 21130041 ± | 603276 ± |
| | | | | | 921451 | 485961 |
| 132 | L-2-Amino-3-(1-pyrazolyl)propanoic | 289.11 | 0.634 | [M − H] − 1 | 17310723 ± | 15429847 ± |
| | acid | | | | 2509618 | 3088778 |
| 133 | 2-Nonenoic acid | 290.15 | 11.431 | [M − H] − 1 | 938653 ± | 16719871 ± |
| | | | | | 192209 | 3136814 |
| 134 | Tranexamic Acid | 292.17 | 11.374 | [M − H] − 1 | 17643544 ± | 33307648 ± |
| | | | | | 2945163 | 1609740 |
| 135 | Medicanine | 295.14 | 3.02 | [M + H] + 1 | 114383667 ± | 27010 ± |
| | | | | | 2429148 | 24846 |
| 136 | Mono-methyl-adipate | 294.11 | 4.03 | [M − H] − 1 | 164606166 ± | 345531 ± |
| | | | | | 10744545 | 28815 |
| 137 | 2-Indolecarboxylic acid | 297.12 | 2.517 | [M + H] + 1 | 46182768 ± | 731536 ± |
| | | | | | 3188500 | 280725 |
| 138 | trans-S-(1-Propenyl)-L-cysteine | 295.10 | 4.136 | [M − H] − 1 | 139677933 ± | 864747 ± |
| | | | | | 6290835 | 119490 |
| 139 | Glutamic acid gamma-methyl ester | 295.10 | 4.243 | [M − H] − 1 | 136680935 ± | 23031084 ± |
| | | | | | 6256752 | 1842721 |
| 140 | hydroxybutyrylglycine | 295.10 | 2.966 | [M − H] − 1 | 11551708 ± | 215419 ± |
| | | | | | 2218830 | 45852 |
| 141 | Acetylhomoserine | 295.10 | 3.436 | [M − H] − 1 | 15606445 ± | 374548 ± |
| | | | | | 1140507 | 73028 |
| 142 | 4-Hydroxycinnamic acid | 298.08 | 7.291 | [M − H] − 1 | 405911 ± | 1277594 ± |
| | | | | | 6763 | 433397 |
| 143 | Enol-phenylpyruvate | 298.08 | 9.173 | [M − H] − 1 | 1357110 ± | 69868 ± |
| | | | | | 607861 | 80016 |
| 144 | 2-Phenylbutyric acid | 300.13 | 4.103 | [M + H] + 1 | 39695 ± | 17431 ± |
| | | | | | 944 | 1723 |
| 145 | 2-methyl-3-phenylpropanoic acid | 298.12 | 9.068 | [M − H] − 1 | 461003 ± | 191382 ± |
| | | | | | 90219 | 197450 |
| 146 | Ethiin | 299.08 | 1.487 | [M − H] − 1 | 15453907 ± | 1797654 ± |
| | | | | | 4516907 | 326811 |
| 147 | 3-Pyridinebutanoic acid | 299.11 | 5.575 | [M − H] − 1 | 247479044 ± | 63752454 ± |
| | | | | | 29561791 | 14251447 |
| 148 | xe2\x80\x8bD-\xe2\x80\x8bXylonic | 300.08 | 3.361 | [M − H] − 1 | 2756231 ± | 49636 ± |
| | acid | | | | 298648 | 3297 |
| 149 | \xe2\x80\x8bL-\xe2\x80\x8bLyxonic acid | 300.08 | 1.772 | [M − H] − 1 | 104447225 ± | 1779717 ± |
| | | | | | 15622140 | 282898 |
| 150 | Arabinonic acid | 300.08 | 2.748 | [M − H] − 1 | 15623924 ± | 104679 ± |
| | | | | | 3130934 | 5285 |
| 151 | 3-(3-Hydroxyphenyl)propanoic acid | 302.11 | 3.318 | [M + H] + 1 | 118424972 ± | 39503 ± |
| | | | | | 28282143 | 3265 |
| 152 | L-3-Phenyllactic acid | 302.11 | 3.093 | [M + H] + 1 | 264477683 ± | 129535 ± |
| | | | | | 5890811 | 5343 |
| 153 | 4-Methoxyphenylacetic acid | 300.10 | 6.158 | [M − H] − 1 | 736422947 ± | 241335 ± |
| | | | | | 9470657 | 318345 |
| 154 | Desaminotyrosine | 300.10 | 6.424 | [M − H] − 1 | 169566041 ± | 115990 ± |
| | | | | | 1708402 | 7464 |
| 155 | 3-(2-Hydroxyphenyl)propanoic acid | 300.10 | 6.667 | [M − H] − 1 | 1048006863 ± | 96793 ± |
| | | | | | 65341318 | 24317 |
| 156 | 4-Hydroxyphenyl-2-propionic acid | 300.10 | 7.58 | [M − H] − 1 | 454523791 ± | 1456452 ± |
| | | | | | 7092875 | 255258 |
| 157 | 2,4,7-Decatrienoic acid | 300.14 | 10.717 | [M − H] − 1 | 2917876 ± | 23001139 ± |
| | | | | | 128098 | 1659257 |
| 158 | 3-Methoxyanthranilate | 303.11 | 5.247 | [M + H] + 1 | 602388 ± | 38221145 ± |
| | | | | | 520287 | 5162239 |
| 159 | 3,5-Dihydroxyphenylacetic acid | 302.08 | 4.991 | [M − H] − 1 | 52800191 ± | 115985 ± |
| | | | | | 718656 | 31839 |
| 160 | 3-hydroxy-5-methoxybenzoic acid | 302.08 | 4.622 | [M − H] − 1 | 36256751 ± | 240292 ± |
| | | | | | 2482980 | 137457 |
| 161 | 2-hydroxy-4-methoxybenzoic acid | 302.08 | 8.429 | [M − H] − 1 | 22207132 ± | 86833441 ± |
| | | | | | 2765128 | 17088956 |
| 162 | cis-4-Decenoic acid | 304.17 | 11.425 | [M − H] − 1 | 2370592 ± | 21248227 ± |
| | | | | | 152654 | 574540 |
| 163 | 9-Oxo-nonanoic acid | 308.16 | 4.817 | [M + H] + 1 | 9520814 ± | 33059 ± |
| | | | | | 3588394 | 14513 |
| 164 | 4-Methylnonanoic acid | 306.18 | 11.823 | [M − H] − 1 | 12847373 ± | 14568161 ± |
| | | | | | 907376 | 1428350 |
| 165 | Gly-Norvaline | 310.15 | 1.318 | [M + H] + 1 | 23644773 ± | 167234 ± |
| | | | | | 16247984 | 23136 |
| 166 | Argininic acid | 311.15 | 0.344 | [M + H] + 1 | 4320252 ± | 281839 ± |
| | | | | | 6922250 | 320176 |
| 167 | 2-Keto-3-deoxy-D-gluconic acid | 312.08 | 3.655 | [M − H] − 1 | 29764174 ± | 77247104 ± |
| | | | | | 6501749 | 9010510 |
| 168 | 4-Methoxycinnamic acid | 312.10 | 10.662 | [M − H] − 1 | 32116255 ± | 232400 ± |
| | | | | | 2003232 | 98866 |
| 169 | 3-(4-Methylphenyl)oxiranecarboxylic | 313.09 | 11.622 | [M − H] − 1 | 2827472 ± | 16637152 ± |
| | acid | | | | 199823 | 1965749 |
| 170 | (Z)-10-Hydroxy-8-decene-4,6-diynoic | 313.09 | 6.476 | [M − H] − 1 | 30002271 ± | 10315091 ± |
| | acid | | | | 138304 | 1734816 |
| 171 | 3-Deoxyarabinohexonic acid | 316.11 | 2.405 | [M + H] + 1 | 351310931 ± | 6215669 ± |
| | | | | | 231028882 | 2120326 |
| 172 | 2-Methyl-3-hydroxy-5-formylpyridine-4- | 315.07 | 8.2 | [M − H] − 1 | 101162767 ± | 26850547 ± |
| | carboxylate | | | | 9157274 | 1483653 |
| 173 | 3,4-Dimethoxybenzoic acid | 316.09 | 5.064 | [M − H] − 1 | 186522028 ± | 120696 ± |
| | | | | | 1420536 | 33735 |
| 174 | 3,5-dimethoxybenzoic acid | 316.09 | 5.412 | [M − H] − 1 | 133874508 ± | 2104614 ± |
| | | | | | 841918 | 674483 |
| 175 | 2-(3-hydroxy-5-methoxyphenyl)acetic | 316.09 | 5.974 | [M − H] − 1 | 30487593 ± | 45370 ± |
| | acid | | | | 417392 | 21447 |
| 176 | 2-hydroxy-2-(3-methoxyphenyl)acetic | 316.09 | 5.809 | [M − H] − 1 | 6557547 ± | 112869 ± |
| | acid | | | | 2121983 | 99540 |
| 177 | 2-hydroxy-2-(4-methoxyphenyl)acetic | 316.09 | 5.491 | [M − H] − 1 | 96542022 ± | 110346 ± |
| | acid | | | | 65077753 | 87059 |
| 178 | 3-(sulfooxy)butanoic acid | 318.04 | 1.911 | [M − H] − 1 | 12219282 ± | 61577 ± |
| | | | | | 4023568 | 3857 |
| 179 | 4-O-Methylgallic acid | 318.07 | 7.051 | [M − H] − 1 | 3039537 ± | 38872274 ± |
| | | | | | 36750 | 5252002 |
| 180 | (Z)-3-Methyl-4-decenoic acid | 318.18 | 11.741 | [M − H] − 1 | 2177468 ± | 4713008 ± |
| | | | | | 206122 | 667305 |
| 181 | 2-Hepteneoylglycine | 319.14 | 6.624 | [M − H] − 1 | 107551942 ± | 54158 ± |
| | | | | | 1217197 | 3408 |
| 182 | Pyroglutamylglycine | 320.10 | 3.259 | [M − H] − 1 | 27875922 ± | 16318665 ± |
| | | | | | 3809371 | 3069977 |
| 183 | 4,6-Dimethylnonanoic acid | 320.20 | 12.19 | [M − H] − 1 | 8577731 ± | 2548002 ± |
| | | | | | 885997 | 2169931 |
| 184 | N-Heptanoylglycine | 323.17 | 3.735 | [M + H] + 1 | 7592240 ± | 10424 ± |
| | | | | | 194459 | 745 |
| 185 | 8-Amino-7-oxononanoic acid | 323.17 | 3.75 | [M + H] + 1 | 21001484 ± | 10221 ± |
| | | | | | 153920 | 650 |
| 186 | Glycyl-Isoleucine | 322.15 | 3.35 | [M − H] − 1 | 5635341 ± | 52481 ± |
| | | | | | 1421294 | 30081 |
| 187 | 5-Hydroxyindoleacetic acid | 325.09 | 6.749 | [M − H] − 1 | 80976425 ± | 47719 ± |
| | | | | | 4891662 | 3762 |
| 188 | 4-Anilino-4-oxobutanoic acid | 327.11 | 6.873 | [M − H] − 1 | 6392717 ± | 235533 ± |
| | | | | | 140654 | 172660 |
| 189 | 3-Dehydro-L-gulonate | 328.08 | 1.927 | [M − H] − 1 | 19632441 ± | 203936 ± |
| | | | | | 1938316 | 8229 |
| 190 | Monoethyl phthalate | 328.09 | 7.029 | [M − H] − 1 | 14467813 ± | 44235 ± |
| | | | | | 356071 | 2248 |
| 191 | 5-(4-hydroxyphenyl)pentanoic acid | 328.13 | 8.105 | [M − H] − 1 | 183963030 ± | 40100 ± |
| | | | | | 15285663 | 3044 |
| 192 | Leucodopachrome | 331.10 | 3.228 | [M + H] + 1 | 526698 ± | 19969 ± |
| | | | | | 420159 | 961 |
| 193 | Dopaquinone | 329.09 | 7.028 | [M − H] − 1 | 1725391 ± | 94224 ± |
| | | | | | 581361 | 80698 |
| 194 | L-Dopaquinone | 329.09 | 5.186 | [M − H] − 1 | 1919681 ± | 117223 ± |
| | | | | | 65458 | 123659 |
| 195 | Metyrosine | 329.09 | 5.53 | [M − H] − 1 | 1512896 ± | 55444 ± |
| | | | | | 881048 | 30166 |
| 196 | Gulonic acid | 330.09 | 4.457 | [M − H] − 1 | 1760105861 ± | 8090776 ± |
| | | | | | 129866114 | 614995 |
| 197 | Gluconic acid | 330.09 | 1.694 | [M − H] − 1 | 141707008 ± | 13400913 ± |
| | | | | | 20621354 | 3618568 |
| 198 | (S)-2-(4-Methoxyphenoxy)propanoic | 332.12 | 3.182 | [M + H] + 1 | 91293128 ± | 448672 ± |
| | acid | | | | 2062863 | 16791 |
| 199 | 2-(3,5-dimethoxyphenyl)acetic acid | 390.13 | 6.593 | [M − H + HAc] − 1 | 88547292 ± | 54152 ± |
| | | | | | 2053671 | 4997 |
| 200 | (S)-Batatic acid | 330.11 | 6.246 | [M − H] − 1 | 231228281 ± | 57698 ± |
| | | | | | 5392957 | 4298 |
| 201 | L-alpha-Amino-1H-pyrrole-1-hexanoic | 332.17 | 2.958 | [M + H] + 1 | 19478437 ± | 17172 ± |
| | acid | | | | 2667138 | 1147 |
| 202 | 2-methyl-4-(sulfooxy)butanoic acid | 332.06 | 4.804 | [M − H] − 1 | 83155581 ± | 39828 ± |
| | | | | | 3838889 | 1554 |
| 203 | 5-(sulfooxy)pentanoic acid | 332.06 | 4.37 | [M − H] − 1 | 36907998 ± | 67315 ± |
| | | | | | 1866491 | 1757 |
| 204 | trans-Dodec-2-enoic acid | 332.20 | 12.151 | [M − H] − 1 | 3414780 ± | 3556575 ± |
| | | | | | 701029 | 3855169 |
| 205 | 2-octenoylglycine | 335.17 | 3.801 | [M + H] + 1 | 3815752 ± | 13985 ± |
| | | | | | 56993 | 831 |
| 206 | 3-octenoylglycine | 335.17 | 3.763 | [M + H] + 1 | 3247986 ± | 16512 ± |
| | | | | | 95378 | 1154 |
| 207 | N-Acetylaminooctanoic acid | 335.17 | 8.436 | [M − H] − 1 | 254623752 ± | 124045 ± |
| | | | | | 38033276 | 7782 |
| 208 | N-(5-Methyl-3-oxohexyl)alanine | 335.17 | 8.352 | [M − H] − 1 | 32078566 ± | 31831 ± |
| | | | | | 869294 | 3475 |
| 209 | R-2-Hydroxy-3-methylbutanoic acid 3- | 336.16 | 6.468 | [M − H] − 1 | 8555654 ± | 117204 ± |
| | Methylbutanoyl | | | | 847309 | 11661 |
| 210 | N-Acetylisoputreanine | 338.18 | 3.003 | [M + H] + 1 | 14410756 ± | 12787 ± |
| | | | | | 680847 | 1274 |
| 211 | D-Tryptophan | 338.13 | 5.705 | [M − H] − 1 | 22623825 ± | 17866954 ± |
| | | | | | 3536393 | 3968802 |
| 212 | Zeanic acid | 339.07 | 7.299 | [M − H] − 1 | 4030088 ± | 92055 ± |
| | | | | | 355673 | 40593 |
| 213 | 3-Indolehydracrylic acid | 339.11 | 7.3 | [M − H] − 1 | 46977371 ± | 2360537 ± |
| | | | | | 1342344 | 426453 |
| 214 | 5-Methoxyindoleacetate | 339.11 | 6.338 | [M − H] − 1 | 32803368 ± | 31758 ± |
| | | | | | 1490625 | 1898 |
| 215 | N-Propionylmethionine | 341.13 | 3.291 | [M + H] + 1 | 3791811 ± | 11764 ± |
| | | | | | 435019 | 981 |
| 216 | 2-Amino-4-hydroxy-6- | 343.10 | 2.643 | [M + H] + 1 | 13643172 ± | 73043 ± |
| | pteridinecarboxylic acid | | | | 11605388 | 89828 |
| 217 | 4-(2-Aminophenyl)-2,4-dioxobutanoic | 343.10 | 3.032 | [M + H] + 1 | 4926716 ± | 14291 ± |
| | acid | | | | 185015 | 2885 |
| 218 | triazolopropionic acid | 341.10 | 9.072 | [M − H] − 1 | 2178392 ± | 84917 ± |
| | | | | | 1048846 | 6258 |
| 219 | 6-Amino-9H-purine-9-propanoic acid | 401.13 | 4.9 | [M − H + HAc] − 1 | 175959193 ± | 256652 ± 1 |
| | | | | | 9078502 | 19357 |
| 220 | 3-Phenylpropionylglycine | 343.14 | 3.686 | [M + H] + 1 | 47607214 ± | 36444 ± |
| | | | | | 1077980 | 14447 |
| 221 | Dihydrolipoate | 402.12 | 5.421 | [M − H + HAc] − 1 | 622208098 ± | 38010336 ± |
| | | | | | 38919379 | 10167513 |
| 222 | 3-(3,4-Dimethoxyphenyl)-2-propenoic | 344.12 | 2.918 | [M + H] + 1 | 17638660 ± | 123198 ± |
| | acid | | | | 859455 | 156293 |
| 223 | 3,4-Dihydroxyphenylvaleric acid | 346.14 | 3.305 | [M + H] + 1 | 25948519 ± | 13913 ± |
| | | | | | 2294846 | 1062 |
| 224 | 2-Methoxy-3-(4- | 344.13 | 6.59 | [M − H] − 1 | 205245407 ± | 52629 ± |
| | methoxyphenyl)propanoic acid | | | | 2715599 | 4856 |
| 225 | Cucurbic acid | 348.19 | 4.795 | [M + H] + 1 | 15736700 ± | 178444 ± |
| | | | | | 4166507 | 41304 |
| 226 | Dihydrojasmonic acid | 348.19 | 4.732 | [M + H] + 1 | 18681975 ± | 261157 ± |
| | | | | | 17465330 | 57845 |
| 227 | 3-hydroxy-2-methyl-2- | 348.05 | 1.76 | [M − H] − 1 | 112459094 ± | 110962 ± |
| | [(sulfooxy)methyl]propanoic acid | | | | 15500812 | 5421 |
| 228 | 9-Methyldodecanoic acid | 348.23 | 12.844 | [M − H] − 1 | 277094 ± | 2426122 ± |
| | | | | | 225202 | 1251444 |
| 229 | 2-hydroxyoct-2-enoylglycine | 349.15 | 10.059 | [M − H] − 1 | 32712097 ± | 95923 ± |
| | | | | | 966406 | 8313 |
| 230 | xi-5-Hydroxydodecanoic acid | 350.21 | 11.513 | [M − H] − 1 | 9600303 ± | 501983 ± |
| | | | | | 233980 | 83437 |
| 231 | Leucyl-Serine | 352.16 | 5.02 | [M − H] − 1 | 35981421 ± | 89528 ± |
| | | | | | 1015854 | 24768 |
| 232 | (2Z)-2-(phenylmethylidene)heptanoic | 352.16 | 5.233 | [M − H] − 1 | 26862526 ± | 69676 ± |
| | acid | | | | 899769 | 29103 |
| 233 | Glycyl-Phenylalanine | 358.15 | 3.029 | [M + H] + 1 | 6341464 ± | 9407 ± |
| | | | | | 2542718 | 1899 |
| 234 | 4-(2-Amino-3-hydroxyphenyl)-2,4- | 357.08 | 3.61 | [M − H] − 1 | 19369899 ± | 127600 ± |
| | dioxobutanoic acid | | | | 1809789 | 17641 |
| 235 | Salsolinol 1-carboxylate | 357.12 | 5.318 | [M − H] − 1 | 160824385 ± | 77612 ± |
| | | | | | 5190562 | 63646 |
| 236 | 2-hydroxyphenylpropionylglycine | 357.12 | 9.578 | [M − H] − 1 | 36220356 ± | 72015 ± |
| | | | | | 8157783 | 29263 |
| 237 | Goshuyic acid | 358.21 | 12.391 | [M − H] − 1 | 4858876 ± | 5590352 ± |
| | | | | | 242757 | 3093250 |
| 238 | 3-(3,4-dihydroxy-5- | 360.08 | 1.919 | [M − H] − 1 | 440578 ± | 12277 ± |
| | methoxyphenyl)oxirane-2-carboxylic | | | | 173758 | 1054 |
| | acid | | | | | |
| 239 | 2,3,4,5,6,7-Hexahydroxyheptanoic acid | 360.10 | 4.269 | [M − H] − 1 | 371551509 ± | 70261443 ± |
| | | | | | 26033213 | 7432650 |
| 240 | 5-(3,4,5-trihydroxyphenyl)pentanoic acid | 362.13 | 1.317 | [M + H] + 1 | 281456 ± | 18467 ± |
| | | | | | 413594 | 4098 |
| 241 | 5-(3,5-dihydroxyphenyl)-4- | 362.13 | 2.919 | [M + H] + 1 | 106945309 ± | 124907 ± |
| | hydroxypentanoic acid | | | | 3957680 | 158460 |
| 242 | b“4-Hydroxy-5-(3,5-dihydroxyphenyl)- | 360.12 | 5.279 | [M − H] − 1 | 287090966 ± | 26714 ± |
| | valeric acid” | | | | 16864207 | 629 |
| 243 | b“4-Hydroxy-(3,4-dihydroxyphenyl)- | 360.12 | 5.514 | [M − H] − 1 | 60249031 ± | 30572 ± |
| | valeric acid” | | | | 1804785 | 1395 |
| 244 | 5-Tetradecenoic acid | 360.23 | 12.667 | [M − H] − 1 | 7452323 ± | 5224125 ± |
| | | | | | 1568864 | 377259 |
| 245 | 5Z-Tetradecenoic acid | 360.23 | 12.829 | [M − H] − 1 | 496992 ± | 3050486 ± |
| | | | | | 395093 | 1241632 |
| 246 | Tsuzuic acid | 360.23 | 12.75 | [M − H] − 1 | 1284972 ± | 5741761 ± |
| | | | | | 605943 | 1385457 |
| 247 | Mevalonic acid-5P | 362.08 | 3.137 | [M − H] − 1 | 1116119 ± | 35967 ± |
| | | | | | 860148 | 2409 |
| 248 | 2-hydroxy-3,4,5-trimethoxybenzoic acid | 362.10 | 5.061 | [M − H] − 1 | 75884655 ± | 117575 ± |
| | | | | | 1386412 | 32863 |
| 249 | 12-Methyltridecanoic acid | 362.24 | 13.168 | [M − H] − 1 | 73882862 ± | 67741975 ± |
| | | | | | 24830050 | 11433835 |
| 250 | 2,6,10-Trimethylundecanoic acid | 362.25 | 13.078 | [M − H] − 1 | 19542478 ± | 407767 ± |
| | | | | | 6376340 | 77424 |
| 251 | Glycyl-Arginine | 365.18 | 8.09 | [M − H] − 1 | 2358321 ± | 36761 ± |
| | | | | | 81605 | 1927 |
| 252 | Salsoline-1-carboxylate | 373.15 | 3.047 | [M + H] + 1 | 6362589 ± | 13105 ± |
| | | | | | 1683811 | 5140 |
| 253 | 3-(3,4,5-Trimethoxyphenyl)propanoic | 374.14 | 8.104 | [M − H] − 1 | 48924645 ± | 38687 ± |
| | acid | | | | 4581942 | 2937 |
| 254 | 2-Carboxy-4-dodecanolide | 376.19 | 11.704 | [M − H] − 1 | 9322395 ± | 131970 ± |
| | | | | | 531703 | 6679 |
| 255 | 13-Methylmyristic acid | 376.26 | 13.381 | [M − H] − 1 | 223486451 ± | 1364882 ± |
| | | | | | 76004756 | 279573 |
| 256 | beta-Alanyl-L-arginine | 379.20 | 9.022 | [M − H] − 1 | 6000099 ± | 100267 ± |
| | | | | | 441874 | 4839 |
| 257 | 3-[3-(Sulfooxy)phenyl]propanoic acid | 380.06 | 5.557 | [M − H] − 1 | 277496418 ± | 88361 ± |
| | | | | | 5803661 | 18966 |
| 258 | 3-[4-(sulfooxy)phenyl]propanoic acid | 380.06 | 5.146 | [M − H] − 1 | 155334437 ± | 51223 ± |
| | | | | | 4568783 | 1624 |
| 259 | Dihydrowyerone acid | 382.15 | 3.675 | [M + H] + 1 | 18988081 ± | 43576 ± |
| | | | | | 5619119 | 4298 |
| 260 | Hydroxynalidixic acid | 382.12 | 9.365 | [M − H] − 1 | 1644804 ± | 3354488 ± |
| | | | | | 59028 | 457929 |
| 261 | 2-{[hydroxy(4-methoxy-1-benzofuran-5- | 383.10 | 10.939 | [M − H] − 1 | 6323472 ± | 118488 ± |
| | yl)methylidene]amino}acetic acid | | | | 14619 | 20811 |
| 262 | Prolyl-Histidine | 386.16 | 5.532 | [M − H] − 1 | 3446983 ± | 54918 ± |
| | | | | | 205990 | 2734 |
| 263 | 7Z,10Z-Hexadecadienoic acid | 386.25 | 12.789 | [M − H] − 1 | 17375911 ± | 2254613 ± |
| | | | | | 6513568 | 385728 |
| 264 | Hypogeic acid | 388.26 | 13.372 | [M − H] − 1 | 2049467 ± | 3205581 ± |
| | | | | | 1062478 | 4187612 |
| 265 | 3-hydroxy-3-(3,4,5- | 390.13 | 6.59 | [M − H] − 1 | 88547292 ± | 53525 ± |
| | trimethoxyphenyl)propanoic acid | | | | 2053671 | 4939 |
| 266 | Isopalmitic acid | 390.28 | 14.227 | [M − H] − 1 | 798566 ± | 6387985 ± |
| | | | | | 908460 | 970468 |
| 267 | Homovanillic acid sulfate | 396.05 | 4.572 | [M − H] − 1 | 49914729 ± | 240546 ± |
| | | | | | 1799733 | 29755 |
| 268 | Dihydrocaffeic acid 3-sulfate | 396.05 | 5.158 | [M − H] − 1 | 9903409 ± | 44531 ± |
| | | | | | 585484 | 1412 |
| 269 | Cinoxacin | 396.10 | 5.28 | [M − H] − 1 | 144214615 ± | 28628 ± |
| | | | | | 17603505 | 1112 |
| 270 | Neuraminic acid | 401.13 | 4.405 | [M − H] − 1 | 895204947 ± | 756226 ± |
| | | | | | 22167360 | 218967 |
| 271 | N-(1-Deoxy-1-fructosyl)serine | 401.13 | 4.742 | [M − H] − 1 | 247932841 ± | 205194 ± |
| | | | | | 4530101 | 152930 |
| 272 | 3-Deoxy-D-glycero-D-galacto-2- | 404.13 | 2.649 | [M + H] + 1 | 284073 ± | 14630952 ± |
| | nonulosonic acid | | | | 225961 | 12664054 |
| 273 | 9E-Heptadecenoic acid | 402.28 | 13.561 | [M − H] − 1 | 6881694 ± | 7020470 ± |
| | | | | | 2783189 | 639263 |
| 274 | Cyclohexaneundecanoic acid | 402.28 | 13.66 | [M − H] − 1 | 11737860 ± | 6261104 ± |
| | | | | | 4834761 | 07290 |
| 275 | (S)-14-Methylhexadecanoic acid | 404.29 | 14.132 | [M − H] − 1 | 1727447 ± | 5804610 ± |
| | | | | | 777515 | 1960928 |
| 276 | 16-Hydroxy hexadecanoic acid | 406.27 | 13.431 | [M − H] − 1 | 41800724 ± | 6689669 ± |
| | | | | | 17205726 | 353350 |
| 277 | (R)-3-Hydroxy-hexadecanoic acid | 406.27 | 12.08 | [M − H] − 1 | 143844245 ± | 7884362 ± |
| | | | | | 16926271 | 2287029 |
| 278 | 3-Hydroxyhexadecanoic acid | 406.27 | 13.173 | [M − H] − 1 | 10222183 ± | 2508112 ± |
| | | | | | 3406065 | 241374 |
| 279 | 5-Hydroxyhexadecanoic acid | 406.27 | 11.916 | [M − H] − 1 | 2615889 ± | 12988902 ± |
| | | | | | 214887 | 3274590 |
| 280 | 7-Hydroxyhexadecanoic acid | 406.27 | 12.695 | [M − H] − 1 | 197513 ± | 5452616 ± |
| | | | | | 66936 | 1190164 |
| 281 | 8-Hydroxyhexadecanoic acid | 406.27 | 11.847 | [M − H] − 1 | 2132446 ± | 4991423 ± |
| | | | | | 223916 | 1237979 |
| 282 | 3,4,5-trihydroxy-6-[(oxolan-2- | 414.15 | 4.637 | [M + H] + 1 | 19217658 ± | 90521 ± |
| | yl)methoxy]oxane-2-carboxylic acid | | | | 4411895 | 6965 |
| 283 | 3,4,5-trihydroxy-6-[(3- | 412.14 | 9.422 | [M − H] − 1 | 19450281 ± | 58025 ± |
| | methylbutanoyl)oxy]oxane-2-carboxylic | | | | 943617 | 2795 |
| | acid | | | | | |
| 284 | Calendic acid | 414.27 | 8.649 | [M + H] + 1 | 102647771 ± | 5100486 ± |
| | | | | | 27477508 | 488517 |
| 285 | Punicic acid | 414.27 | 8.461 | [M + H] + 1 | 48950252 ± | 2630668 ± |
| | | | | | 19169432 | 1109662 |
| 286 | Linolenelaidic acid | 414.27 | 7.48 | [M + H] + 1 | 3003039 ± | 27593590 ± |
| | | | | | 923342 | 4955418 |
| 287 | 3,4,5-trihydroxy-6-[(3- | 414.12 | 8.168 | [M − H] − 1 | 706597767 ± | 136155 ± |
| | hydroxybutanoyl)oxy]oxane-2- | | | | 24989472 | 54460 |
| | carboxylic acid | | | | | |
| 288 | Tyrosyl-Valine | 414.18 | 4.492 | [M − H] − 1 | 1247190 ± | 24904 ± |
| | | | | | 658172 | 4829 |
| 289 | Bovinic acid | 416.29 | 9.488 | [M + H] + 1 | 562037780 ± | 2307000 ± |
| | | | | | 83001976 | 798698 |
| 290 | (9E,11E)-Octadecadienoic acid | 416.29 | 9.822 | [M + H] + 1 | 174914828 ± | 3328836 ± |
| | | | | | 21737726 | 4605771 |
| 291 | Dihomolinoleic acid | 416.29 | 9.712 | [M + H] + 1 | 488647177 ± | 1620981 ± |
| | | | | | 80759240 | 160741 |
| 292 | 5-Octadecynoic acid | 416.29 | 11.458 | [M + H] + 1 | 5607810 ± | 57252158 ± |
| | | | | | 3308500 | 8867798 |
| 293 | 6Z,9Z-octadecadienoic acid | 416.29 | 13.896 | [M + H] + 1 | 67451068 ± | 665948 ± |
| | | | | | 30079106 | 344192 |
| 294 | Octadecadienoate | 416.29 | 9.615 | [M + H] + 1 | 259619642 ± | 1555275 ± |
| | | | | | 130161609 | 347600 |
| 295 | (10E,12Z)-Octadecadienoic acid | 414.28 | 13.498 | [M − H] − 1 | 57038511 ± | 33771010 ± |
| | | | | | 23912024 | 3873450 |
| 296 | Mangiferic acid | 414.28 | 13.414 | [M − H] − 1 | 676271339 ± | 824087609 ± |
| | | | | | 266834677 | 110539718 |
| 297 | Elaidic acid | 416.29 | 13.89 | [M − H] − 1 | 345423297 ± | 589882789 ± |
| | | | | | 155279070 | 97391985 |
| 298 | Vaccenic acid | 416.29 | 13.978 | [M − H] − 1 | 123869005 ± | 27280370 ± |
| | | | | | 54460008 | 5217242 |
| 299 | 16-Methylheptadecanoic acid | 418.31 | 14.424 | [M − H] − 1 | 10439852 ± | 128642288 ± |
| | | | | | 3930146 | 34500866 |
| 300 | xanthurenic acid 8-O-sulfate | 419.03 | 10.048 | [M − H] − 1 | 517087 ± | 205207 ± |
| | | | | | 514753 | 88643 |
| 301 | 12-hydroxyheptadecanoic acid | 420.29 | 12.39 | [M − H] − 1 | 20657364 ± | 171209 ± |
| | | | | | 2632544 | 72238 |
| 302 | Orotidine | 422.10 | 3.158 | [M − H] − 1 | 8172693 ± | 30250 ± |
| | | | | | 6969485 | 1926 |
| 303 | Porric acid | 422.10 | 5.508 | [M − H] − 1 | 36755714 ± | 67250 ± |
| | | | | | 3624668 | 15454 |
| 304 | 3,5-Dihydroxyphenylvaleric acid sulfate | 424.08 | 5.899 | [M − H] − 1 | 72314336 ± | 47565 ± |
| | | | | | 3330294 | 1244 |
| 305 | 3,4,5-trihydroxy-6-[(4-hydroxy-3- | 445.16 | 3.859 | [M + NH4] + 1 | 2047231 ± | 96163 ± |
| | methylbut-2-enoyl)oxy]oxane-2- | | | | 27120 | 3734 |
| | carboxylic acid | | | | | |
| 306 | 4-Hydroxyproline galactoside | 487.17 | 5.655 | [M − H + HAc] − 1 | 131390623 ± | 426038 ± |
| | | | | | 5723075 | 109801 |
| 307 | 17-Hydroxylinolenic acid | 428.26 | 11.762 | [M − H] − 1 | 2567010 ± | 5148556 ± |
| | | | | | 1301141 | 134212 |
| 308 | (Z)-13-Oxo-9-octadecenoic acid | 430.27 | 12.372 | [M − H] − 1 | 4527380 ± | 8539429 ± |
| | | | | | 934039 | 1171168 |
| 309 | Avenoleic acid | 430.27 | 12.1 | [M − H] − 1 | 29642015 ± | 92203928 ± |
| | | | | | 2317532 | 33278090 |
| 310 | 12-Hydroxy-8,10-octadecadienoic acid | 430.27 | 12.67 | [M − H] − 1 | 916569 ± | 6382616 ± |
| | | | | | 1164755 | 1198719 |
| 311 | (10E,12Z)-9-HODE | 430.27 | 12.474 | [M − H] − 1 | 5124568 ± | 11353262 ± |
| | | | | | 898704 | 4357069 |
| 312 | 13-HODE | 430.27 | 12.167 | [M − H] − 1 | 26291952 ± | 77040779 ± |
| | | | | | 1780901 | 12269888 |
| 313 | alpha-Dimorphecolic acid | 430.27 | 11.929 | [M − H] − 1 | 6069564 ± | 21720795 ± |
| | | | | | 2188196 | 3055423 |
| 314 | 9,10-Epoxyoctadecenoic acid | 430.27 | 11.858 | [M − H] − 1 | 4797819 ± | 11283123 ± |
| | | | | | 1032732 | 2319925 |
| 315 | 12,13-EpOME | 430.27 | 12.875 | [M − H] − 1 | 2267111 ± | 4829389 ± |
| | | | | | 2281884 | 847226 |
| 316 | Phenethylamine glucuronide | 430.27 | 12.257 | [M − H] − 1 | 9251186 ± | 32181745 ± |
| | | | | | 315903 | 41126080 |
| 317 | 3-Oxooctadecanoic acid | 432.29 | 12.823 | [M − H] − 1 | 1572760 ± | 2572358 ± |
| | | | | | 174555 | 511016 |
| 318 | 9-Oxooctadecanoic acid | 432.29 | 12.469 | [M − H] − 1 | 20085576 ± | 11604607 ± |
| | | | | | 4030884 | 1765007 |
| 319 | 10-Oxooctadecanoic acid | 432.29 | 12.339 | [M − H] − 1 | 510669565 ± | 9396774 ± |
| | | | | | 89374880 | 8198094 |
| 320 | 11-Oxooctadecanoic acid | 432.29 | 12.277 | [M − H] − 1 | 122641516 ± | 22288090 ± |
| | | | | | 17057886 | 15318462 |
| 321 | 5-Hexyltetrahydro-2-furanoctanoic acid | 432.29 | 12.883 | [M − H] − 1 | 399730 ± | 1123125 ± |
| | | | | | 64764 | 334858 |
| 322 | (R)-3-Hydroxy-Octadecanoic acid | 434.30 | 12.56 | [M − H] − 1 | 1284577242 ± | 7545156 ± |
| | | | | | 305296771 | 4668860 |
| 323 | xi-10-Hydroxyoctadecanoic acid | 434.30 | 12.695 | [M − H] − 1 | 27938700 ± | 2261349 ± |
| | | | | | 5715126 | 91408 |
| 324 | Pyrogallol-2-O-glucuronide | 436.10 | 11.189 | [M − H] − 1 | 539805 ± | 3253620 ± |
| | | | | | 160230 | 562979 |
| 325 | 6-(2,4-dihydroxyphenoxy)-3,4,5- | 436.10 | 11.349 | [M − H] − 1 | 2213769 ± | 21151840 ± |
| | trihydroxyoxane-2-carboxylic acid | | | | 719244 | 2364104 |
| 326 | 6-(2,5-dihydroxyphenoxy)-3,4,5- | 436.10 | 11.63 | [M − H] − 1 | 2068500 ± | 22519723 ± |
| | trihydroxyoxane-2-carboxylic acid | | | | 373176 | 1015590 |
| 327 | 6-(3,4-dihydroxyphenoxy)-3,4,5- | 436.10 | 11.46 | [M − H] − 1 | 3247448 ± | 29391873 ± |
| | trihydroxyoxane-2-carboxylic acid | | | | 664244 | 2501473 |
| 328 | Porric acid A | 436.11 | 5.843 | [M − H] − 1 | 92312578 ± | 35725 ± |
| | | | | | 3795837 | 431 |
| 329 | Cis-8,11,14,17-Eicosatetraenoic acid | 440.29 | 11.266 | [M + H] + 1 | 1733568 ± | 53280633 ± |
| | | | | | 489736 | 41031354 |
| 330 | 2-(3,4-Dihydroxybenzoyloxy)-4,6- | 440.07 | 13.6 | [M − H] − 1 | 356155 ± | 82829 ± |
| | dihydroxybenzoate | | | | 250378 | 2856 |
| 331 | 5-(3,4-dihydroxyphenyl)-4- | 440.08 | 4.794 | [M − H] − 1 | 176423351 ± | 34249 ± |
| | (sulfooxy)pentanoic acid | | | | 2727594 | 1355 |
| 332 | 3,4-dihydroxy-2-(4-hydroxy-3,7- | 440.18 | 0.955 | [M − H] − 1 | 21206815 ± | 535907 ± |
| | dimethylocta-2,6-dien-1-yl)benzoic acid | | | | 18068363 | 30832 |
| 333 | 3,4-dihydroxy-2-[(2Z)-4-hydroxy-3-(4- | 440.18 | 0.91 | [M − H] − 1 | 34747475 ± | 1427681 ± |
| | methylpent-3-en-1-yl)but-2-en-1- | | | | 7183172 | 727074 |
| | yl]benzoic acid | | | | | |
| 334 | b“5-Carboxy-gamma-chromanol” | 442.23 | 5.534 | [M + H] + 1 | 39410744 ± | 232039 ± |
| | | | | | 770524 | 10693 |
| 335 | 5,8,11-Eicosatrienoic acid | 440.29 | 13.577 | [M − H] − 1 | 10228924 ± | 16089071 ± |
| | | | | | 5254138 | 2598430 |
| 336 | Corchorifatty acid A | 442.23 | 3.328 | [M − H] − 1 | 39191074 ± | 16582274 ± |
| | | | | | 8328846 | 2020810 |
| 337 | Eicosadienoic acid | 442.31 | 13.969 | [M − H] − 1 | 1632999 ± | 5330577 ± |
| | | | | | 1028883 | 825514 |
| 338 | 3,4,5-trihydroxy-6-{[3-hydroxy-2- | 446.14 | 3.287 | [M + H] + 1 | 14686973 ± | 144711 ± |
| | (hydroxymethyl)-2- | | | | 385197 | 36494 |
| | methylpropanoyl]oxy}oxane-2- | | | | | |
| | carboxylic acid | | | | | |
| 339 | (R)-2-Hydroxysterculic acid | 444.29 | 12.867 | [M − H] − 1 | 2656442 ± | 3269807 ± |
| | | | | | 1286306 | 212336 |
| 340 | 8(R)-Hydroperoxylinoleic acid | 446.27 | 12.188 | [M − H] − 1 | 12593976 ± | 37783556 ± |
| | | | | | 1999137 | 11286390 |
| 341 | (\xc2\xb1)-(E)-13-Hydroxy-10-oxo-11- | 446.27 | 12.051 | [M − H] − 1 | 2831245 ± | 8969604 ± |
| | octadecenoic acid | | | | 905725 | 2221456 |
| 342 | Beta-D-Glucopyranuronic acid | 448.10 | 12.379 | [M − H] − 1 | 317677 ± | 8922364 ± |
| | | | | | 165203 | 674867 |
| 343 | 3,4,5-trihydroxy-6-(3- | 448.10 | 11.648 | [M − H] − 1 | 422340 ± | 10693456 ± |
| | hydroxybenzoyloxy)oxane-2-carboxylic | | | | 54500 | 5702913 |
| | acid | | | | | |
| 344 | 9,10-Epoxyoctadecanoic acid | 448.28 | 11.641 | [M − H] − 1 | 87004989 ± | 1374409 ± |
| | | | | | 3973815 | 184155 |
| 345 | 12,13-DHOME | 448.28 | 12.358 | [M − H] − 1 | 1026380 ± | 2297055 ± |
| | | | | | 608068 | 1494177 |
| 346 | 9,10-DHOME | 448.28 | 12.418 | [M − H] − 1 | 1898753 ± | 3003796 ± |
| | | | | | 312906 | 1371028 |
| 347 | (+)-15,16-Dihydroxyoctadecanoic acid | 452.31 | 7.996 | [M + H] + 1 | 182080968 ± | 1005144 ± |
| | | | | | 49497922 | 477658 |
| 348 | (9S,10S)-9,10-dihydroxyoctadecanoate | 452.31 | 7.597 | [M + H] + 1 | 33056391 ± | 961508 ± |
| | | | | | 2714072 | 142134 |
| 349 | Valproic acid glucuronide | 456.20 | 3.676 | [M + H] + 1 | 32799856 ± | 123805 ± |
| | | | | | 3113321 | 7280 |
| 350 | 2-Hydroxy-6-tridecylbenzoic acid | 454.27 | 12.179 | [M − H] − 1 | 371798 ± | 10320632 ± |
| | | | | | 232645 | 7951616 |
| 351 | 19(S)-HETE | 454.27 | 12.35 | [M − H] − 1 | 267167 ± | 11165932 ± |
| | | | | | 38896 | 1027869 |
| 352 | 10-HETE | 454.27 | 12.615 | [M − H] − 1 | 203539 ± | 41648687 ± |
| | | | | | 48719 | 2147758 |
| 353 | 13-HETE | 454.27 | 12.428 | [M − H] − 1 | 437565 ± | 15692639 ± |
| | | | | | 96921 | 3729459 |
| 354 | 12 Hydroxy arachidonic acid | 454.27 | 12.282 | [M − H] − 1 | 222454 ± | 11264070 ± |
| | | | | | 74787 | 2210161 |
| 355 | 12S-hydroxy-5E,8Z,10Z,14Z- | 454.27 | 12.451 | [M − H] − 1 | 100756 ± | 4218983 ± |
| | eicosatetraenoic acid | | | | 17609 | 1545730 |
| 356 | Lepidimoic acid | 458.14 | 3.472 | [M + H] + 1 | 24066028 ± | 19662 ± |
| | | | | | 5739940 | 1735 |
| 357 | 3,4-Dimethyl-5-pentyl-2-furannonanoic | 456.29 | 12.047 | [M − H] − 1 | 23661841 ± | 256504 ± |
| | acid | | | | 1984650 | 95569 |
| 358 | Corchorifatty acid F | 463.29 | 12.897 | [M − H] − 1 | 8172175 ± | 135246 ± |
| | | | | | 2165706 | 6453 |
| 359 | 9,12,13-TriHOME | 464.28 | 9.733 | [M − H] − 1 | 54386843 ± | 592064 ± |
| | | | | | 3628022 | 27726 |
| 360 | 9,10,13-TriHOME | 464.28 | 9.812 | [M − H] − 1 | 111662018 ± | 1929528 ± |
| | | | | | 7179078 | 597563 |
| 361 | 4,8,12,15,19-Docosapentaenoic acid | 464.29 | 13.374 | [M − H] − 1 | 3022443 ± | 2386925 ± |
| | | | | | 1435794 | 484525 |
| 362 | 9,10,13-Trihydroxystearic acid | 466.29 | 10.34 | [M − H] − 1 | 15563703 ± | 109258 ± |
| | | | | | 377280 | 6956 |
| 363 | Prostaglandin B2 | 468.25 | 11.539 | [M − H] − 1 | 10813788 ± | 184849 ± |
| | | | | | 1406868 | 117650 |
| 364 | 3,4,5-trihydroxy-6-{[(2E)-3-(3- | 474.12 | 12.01 | [M − H] − 1 | 285412 ± | 10033224 ± |
| | hydroxyphenyl)prop-2- | | | | 36657 | 561452 |
| | enoyl]oxy}oxane-2-carboxylic acid | | | | | |
| 365 | 3,4,5-trihydroxy-6-{[(2E)-3-(4- | 474.12 | 11.83 | [M − H] − 1 | 561451 ± | 15328466 ± |
| | hydroxyphenyl)prop-2- | | | | 154877 | 2978586 |
| | enoyl]oxy}oxane-2-carboxylic acid | | | | | |
| 366 | 3,4,5-trihydroxy-6-{[3-(3- | 474.12 | 12.27 | [M − H] − 1 | 230347 ± | 4180602 ± |
| | hydroxyphenyl)prop-2- | | | | 28396 | 2422104 |
| | enoyl]oxy}oxane-2-carboxylic acid | | | | | |
| 367 | 3,4,5-trihydroxy-6-[3-(4- | 478.18 | 3.776 | [M + H] + 1 | 6144013 ± | 8744 ± |
| | methoxyphenyl)propoxy]oxane-2- | | | | 619614 | 750 |
| | carboxylic acid | | | | | |
| 368 | 13-Hydroxy-9-methoxy-10-oxo-11- | 476.28 | 12.356 | [M − H] − 1 | 707597 ± | 11725314 ± |
| | octadecenoic acid | | | | 99637 | 9946207 |
| 369 | 2-Hydroxy-6-pentadecylbenzoic acid | 482.30 | 11.881 | [M − H] − 1 | 12770057 ± | 151740 ± |
| | | | | | 1591691 | 2572 |
| 370 | 4-Methylumbelliferone glucuronide | 486.12 | 11.739 | [M − H] − 1 | 749110 ± | 27793207 ± |
| | | | | | 44634 | 946668 |
| 371 | Cryptochlorogenic acid | 488.13 | 11.943 | [M − H] − 1 | 1338251 ± | 52406067 ± |
| | | | | | 89179 | 6324506 |
| 372 | 5-Caffeoylquinic acid | 488.13 | 11.81 | [M − H] − 1 | 1001686 ± | 47163602 ± |
| | | | | | 629003 | 2046485 |
| 373 | 3,4,5-trihydroxy-6-{[3-(3- | 488.13 | 11.33 | [M − H] − 1 | 764298 ± | 65035070 ± |
| | methoxyphenyl)prop-2- | | | | 193256 | 4754398 |
| | enoyl]oxy}oxane-2-carboxylic acid | | | | | |
| 374 | 3,4,5-trihydroxy-6-{[(2E)-3-(3- | 488.13 | 12.013 | [M − H] − 1 | 358182 ± | 37143083 ± |
| | methoxyphenyl)prop-2- | | | | 28055 | 645807 |
| | enoyl]oxy}oxane-2-carboxylic acid | | | | | |
| 375 | 3,4,5-trihydroxy-6-{[(2E)-2- | 488.13 | 11.865 | [M − H] − 1 | 3288892 ± | 164304226 ± |
| | (hydroxymethyl)-3-phenylprop-2- | | | | 904240 | 19453671 |
| | enoyl]oxy}oxane-2-carboxylic acid | | | | | |
| 376 | 3,4,5-trihydroxy-6-{[(2E)-3-(3- | 488.13 | 11.716 | [M − H] − 1 | 5244846 ± | 257754514 ± |
| | hydroxyphenyl)-2-methylprop-2- | | | | 1462206 | 29782660 |
| | enoyl]oxy}oxane-2-carboxylic acid | | | | | |
| 377 | 3,4,5-trihydroxy-6-{[(2E)-3-(4- | 488.13 | 11.641 | [M − H] − 1 | 1183413 ± | 110937452 ± |
| | hydroxyphenyl)-2-methylprop-2- | | | | 344044 | 11980258 |
| | enoyl]oxy}oxane-2-carboxylic acid | | | | | |
| 378 | 3,4,5-trihydroxy-6-{[(2E)-3-(2- | 488.13 | 11.572 | [M − H] − 1 | 552018 ± | 52501894 ± |
| | hydroxyphenyl)-2-methylprop-2- | | | | 248208 | 2188003 |
| | enoyl]oxy}oxane-2-carboxylic acid | | | | | |
| 379 | 3,4,5-trihydroxy-6-{[3-(4- | 488.13 | 11.208 | [M − H] − 1 | 764298 ± | 11449342 ± |
| | methoxyphenyl)prop-2- | | | | 193256 | 2788799 |
| | enoyl]oxy}oxane-2-carboxylic acid | | | | | |
| 380 | Sulindac | 490.12 | 12.5 | [M − H] − 1 | 248999 ± | 1067937 ± |
| | | | | | 124486 | 44336 |
| 381 | 3,4,5-trihydroxy-6-[5-hydroxy-2- | 492.16 | 7.476 | [M + H] + 1 | 415073 ± | 32153032 ± |
| | methoxy-4-(prop-2-en-1- | | | | 50302 | 1481907 |
| | yl)phenoxy]oxane-2-carboxylic acid | | | | | |
| 382 | 3,4,5-trihydroxy-6-[5-hydroxy-4- | 492.16 | 6.333 | [M + H] + 1 | 265877 ± | 30343221 ± |
| | methoxy-2-(prop-2-en-1- | | | | 117576 | 1013625 |
| | yl)phenoxy]oxane-2-carboxylic acid | | | | | |
| 383 | 3,4,5-trihydroxy-6-{[3-(3- | 492.16 | 6.393 | [M + H] + 1 | 283221 ± | 80304902 ± |
| | methoxyphenyl)propanoyl]oxy}oxane-2- | | | | 98823 | 15929303 |
| | carboxylic acid | | | | | |
| 384 | 3,4,5-trihydroxy-6-{[2-(4- | 490.15 | 11.461 | [M − H] − 1 | 231668 ± | 2885292 ± |
| | methoxyphenyl)propanoyl]oxy}oxane-2- | | | | 24742 | 641625 |
| | carboxylic acid | | | | | |
| 385 | Tetracosahexaenoic acid | 492.32 | 7.847 | [M + H] + 1 | 7220101994 ± | 1726046 ± |
| | | | | | 144799958 | 120207 |
| 386 | 6,9,12,15,18,21-Tetracosahexaenoic acid | 492.32 | 6.284 | [M + H] + 1 | 171476795 ± | 201112 ± |
| | | | | | 42496040 | 263735 |
| 387 | Tetracosahexaenoic acid, n-3 | 492.32 | 5.731 | [M + H] + 1 | 275527792 ± | 517583 ± |
| | | | | | 16577519 | 407316 |
| 388 | 5-hydroxy-2-{[1-hydroxy-3-(4-hydroxy- | 493.13 | 6.238 | [M − H] − 1 | 83824095 ± | 53113 ± |
| | 3-methoxyphenyl)prop-2-en-1- | | | | 5817825 | 7086 |
| | ylidene]amino}-4-methoxybenzoic acid | | | | | |
| 389 | Rosmarinic acid | 494.12 | 6.676 | [M − H] − 1 | 69856616 ± | 37698 ± |
| | | | | | 5550646 | 3154 |
| 390 | Feruloyl C1-glucuronide | 504.13 | 11.828 | [M − H] − 1 | 1150529 ± | 4030880 ± |
| | | | | | 12608 | 859188 |
| 391 | Isoferuloyl C1-glucuronide | 504.13 | 10.569 | [M − H] − 1 | 761604 ± | 11722683 ± |
| | | | | | 15570 | 1247781 |
| 392 | 3,4,5-trihydroxy-6-{[3-(4-hydroxy-3- | 504.13 | 10.791 | [M − H] − 1 | 366531 ± | 4752867 ± |
| | methoxyphenyl)prop-2- | | | | 48454 | 340214 |
| | enoyl]oxy}oxane-2-carboxylic acid | | | | | |
| 393 | 3,4,5-trihydroxy-6-{[3-(3-hydroxy-5- | 504.13 | 11.361 | [M − H] − 1 | 978673 ± | 19126186 ± |
| | methoxyphenyl)prop-2- | | | | 64639 | 674346 |
| | enoyl]oxy}oxane-2-carboxylic acid | | | | | |
| 394 | 3,4,5-trihydroxy-6-{[(2E)-3-(4-hydroxy- | 504.13 | 11.203 | [M − H] − 1 | 541613 ± | 10096751 ± |
| | 3-methoxyphenyl)prop-2- | | | | 25396 | 2230534 |
| | enoyl]oxy}oxane-2-carboxylic acid | | | | | |
| 395 | 3,4,5-trihydroxy-6-{[3-(2-hydroxy-4- | 504.13 | 10.956 | [M − H] − 1 | 403501 ± | 9765280 ± |
| | methoxyphenyl)prop-2- | | | | 13037 | 2718087 |
| | enoyl]oxy}oxane-2-carboxylic acid | | | | | |
| 396 | 3,4,5-trihydroxy-6-{[3-(4- | 504.13 | 10.69 | [M − H] − 1 | 451786 ± | 5949088 ± |
| | methoxyphenyl)-2- | | | | 31638 | 1085216 |
| | oxopropanoyl]oxy}oxane-2-carboxylic | | | | | |
| | acid | | | | | |
| 397 | (3R)-3-(tetradecanoyloxy)-4- | 507.35 | 10.39 | [M + H] + 1 | 188234605 ± | 871188 ± |
| | (trimethylazaniumyl)butanoate | | | | 73194844 | 199932 |
| 398 | Ethyl gallate 3-glucuronide | 508.12 | 8.985 | [M − H] − 1 | 105752943 ± | 163314 ± |
| | | | | | 45846541 | 7882 |
| 399 | 2-(10-Heptadecenyl)-6-hydroxybenzoic | 508.32 | 13.021 | [M − H] − 1 | 12794494 ± | 97035 ± |
| | acid | | | | 4306336 | 3888 |
| 400 | 12b-Hydroxy-5b-cholanoic acid | 510.33 | 12.428 | [M − H] − 1 | 641446664 ± | 949703 ± |
| | | | | | 189084164 | 735439 |
| 401 | Allolithocholic acid | 510.33 | 12.025 | [M − H] − 1 | 115231454 ± | 359177 ± |
| | | | | | 38319237 | 362857 |
| 402 | 7a-Hydroxy-5b-cholanic acid | 510.33 | 11.916 | [M − H] − 1 | 1156055 ± | 250890 ± |
| | | | | | 338435 | 247541 |
| 403 | 6-[(4,7-dihydroxy-2,2-dimethyl-3,4- | 520.16 | 12.006 | [M − H] − 1 | 1870162 ± | 11915130 ± |
| | dihydro-2H-1-benzopyran-5-yl)oxy]- | | | | 475219 | 2335910 |
| | 3,4,5-trihydroxyoxane-2-carboxylic acid | | | | | |
| 404 | Treprostinil | 524.28 | 12.31 | [M − H] − 1 | 980525 ± | 8520699 ± |
| | | | | | 523501 | 451539 |
| 405 | b“D8-Merulinic acid A” | 524.31 | 11.366 | [M − H] − 1 | 541158745 ± | 449488 ± |
| | | | | | 374961606 | 279265 |
| 406 | 7-Hydroxy-3-oxocholanoic acid | 524.31 | 11.592 | [M − H] − 1 | 5235781 ± | 169193 ± |
| | | | | | 3448206 | 8232 |
| 407 | 11-Hydroperoxy-H4-neuroprostane | 526.26 | 11.574 | [M − H] − 1 | 331036 ± | 100458 ± |
| | | | | | 85161 | 838 |
| 408 | 3a,12b-Dihydroxy-5b-cholanoic acid | 526.33 | 11.944 | [M − H] − 1 | 1812605216 ± | 4679585 ± |
| | | | | | 192418223 | 378889 |
| 409 | 3b,12a-Dihydroxy-5a-cholanoic acid | 526.33 | 10.534 | [M − H] − 1 | 679106438 ± | 388461 ± |
| | | | | | 40615214 | 78335 |
| 410 | 3b,7a-Dihydroxy-5b-cholanoic acid | 526.33 | 11.778 | [M − H] − 1 | 548961777 ± | 2777820 ± |
| | | | | | 30873343 | 251814 |
| 411 | 3a,7a-Dihydroxycholanoic acid | 526.33 | 10.902 | [M − H] − 1 | 660581393 ± | 322672 ± |
| | | | | | 34926669 | 217922 |
| 412 | Allodeoxycholic acid | 526.33 | 11.604 | [M − H] − 1 | 44284334 ± | 506183 ± |
| | | | | | 852858 | 327197 |
| 413 | Allochenodeoxycholic acid | 526.33 | 11.871 | [M − H] − 1 | 29068727 ± | 186069 ± |
| | | | | | 3682524 | 5434 |
| 414 | Murocholic acid | 526.33 | 11.249 | [M − H] − 1 | 148446058 ± | 287273 ± |
| | | | | | 9847095 | 26392 |
| 415 | 7a,12b-dihydroxy-5b-Cholan-24-oic acid | 526.33 | 11.33 | [M − H] − 1 | 932019319 ± | 436568 ± |
| | | | | | 348382725 | 286077 |
| 416 | 3beta,12beta-Dihydroxy-5beta-cholanoic | 526.33 | 12.199 | [M − H] − 1 | 232158793 ± | 476448 ± |
| | acid | | | | 10792209 | 7844 |
| 417 | Sinapinic acid-O-glucuronide isomer | 534.14 | 11.937 | [M − H] − 1 | 1217503 ± | 3553889 ± |
| | | | | | 81133 | 730667 |
| 418 | 3,4,5-trihydroxy-6-{[6-(3-hydroxyprop- | 534.14 | 11.325 | [M − H] − 1 | 1832391 ± | 142882837 ± |
| | 1-en-1-yl)-5-methoxy-2H-1,3- | | | | 1084795 | 7978855 |
| | benzodioxol-4-yl]oxy}oxane-2- | | | | | |
| | carboxylic acid | | | | | |
| 419 | 3,4,5-trihydroxy-6-[2-hydroxy-3,4- | 534.14 | 11.57 | [M − H] − 1 | 634692 ± | 53948195 ± |
| | dimethoxy-5-(3-oxoprop-1-en-1- | | | | 486133 | 4992807 |
| | yl)phenoxy]oxane-2-carboxylic acid | | | | | |
| 420 | b“5-(3,4,5-Trihydroxyphenyl)-gamma- | 534.14 | 11.654 | [M − H] − 1 | 387193 ± | 51877403 ± |
| | valerolactone-3-O-glucuronide” | | | | 106043 | 14353027 |
| 421 | b“5-(3,4,5-Trihydroxyphenyl)-gamma- | 536.19 | 4.204 | [M + H] + 1 | 25890202 ± | 146487 ± |
| | valerolactone-4-O-glucuronide” | | | | 1901296 | 4740 |
| 422 | b“4-Hydroxy-5-(3,4-dihydroxyphenyl)- | 536.15 | 11.452 | [M − H] − 1 | 311804 ± | 39471028 ± |
| | valeric acid-O-glucuronide” | | | | 56347 | 2427874 |
| 423 | 6-{[5-(3,4-dihydroxyphenyl)-4- | 536.15 | 11.878 | [M − H] − 1 | 193855 ± | 18955563 ± |
| | hydroxypentanoyl]oxy}-3,4,5- | | | | 22678 | 1049628 |
| | trihydroxyoxane-2-carboxylic acid | | | | | |
| 424 | 3,4,5-trihydroxy-6-[2-hydroxy-3,4- | 536.15 | 11.51 | [M − H] − 1 | 343919 ± | 35256359 ± |
| | dimethoxy-5-(3- | | | | 116260 | 3190656 |
| | oxopropyl)phenoxy]oxane-2-carboxylic | | | | | |
| | acid | | | | | |
| 425 | 7-Ketodeoxycholic acid | 542.32 | 4.197 | [M + H] + 1 | 157854029 ± | 156172 ± |
| | | | | | 9462565 | 5053 |
| 426 | 3,7-Dihydroxy-12-oxocholanoic acid | 542.32 | 3.838 | [M + H] + 1 | 9016731 ± | 10773 ± |
| | | | | | 878392 | 1084 |
| 427 | 1b,3a,12a-Trihydroxy-5b-cholanoic acid | 542.32 | 10.137 | [M − H] − 1 | 167611063 ± | 114235 ± |
| | | | | | 4327022 | 4658 |
| 428 | 1b,3a,7a-Trihydroxy-5b-cholanoic acid | 542.32 | 11.148 | [M − H] − 1 | 544907160 ± | 3155128 ± |
| | | | | | 4565684 | 56662 |
| 429 | 3alpha,7alpha,12beta-Trihydroxy-5beta- | 542.32 | 9.526 | [M − H] − 1 | 1241848325 ± | 64094 ± |
| | cholanoic acid | | | | 21099752 | 2673 |
| 430 | 3a,4b,7a-Trihydroxy-5b-cholanoic acid | 542.32 | 10.707 | [M − H] − 1 | 3495175556 ± | 674030 ± |
| | | | | | 579880 | 577596 |
| 431 | 3a,4b,12a-Trihydroxy-5b-cholanoic acid | 542.32 | 9.706 | [M − H] − 1 | 195680988 ± | 224977 ± |
| | 12-Ketodeoxycholic acid | | | | 10657635 | 9852 |
| 432 | 1,3,12-Trihydroxycholan-24-oic acid | 542.32 | 8.65 | [M − H] − 1 | 925432007 ± | 88584 ± |
| | | | | | 59000243 | 12829 |
| 433 | 3b,7a,12a-Trihydroxy-5a-Cholanoic acid | 542.32 | 9.97 | [M − H] − 1 | 27588201 ± | 429960 ± |
| | | | | | 4722587 | 32149 |
| 434 | 3a,7b,12b-Trihydroxy-5b-cholanoic acid | 542.32 | 8.09 | [M − H] − 1 | 35540636 ± | 38071 ± |
| | | | | | 3548529 | 1995 |
| 435 | 3b,7b,12a-Trihydroxy-5b-cholanoic acid | 542.32 | 8.82 | [M − H] − 1 | 25513288 ± | 75397 ± |
| | | | | | 9016311 | 3160 |
| 436 | 2b,3a,7a-Trihydroxy-5b-cholanoic acid | 542.32 | 8.973 | [M − H] − 1 | 21080013 ± | 118675 ± |
| | | | | | 14979217 | 8489 |
| 437 | Trovafloxacin | 552.16 | 5.011 | [M + H] + 1 | 25987908 ± | 16523986 ± |
| | | | | | 5996264 | 1033800 |
| 438 | 3,4,5-trihydroxy-6-{4-hydroxy-2-[(2E)- | 550.14 | 10.286 | [M − H] − 1 | 48373249 ± | 18790255 ± |
| | 3-phenylprop-2-enoyl]phenoxy}oxane-2- | | | | 12447522 | 2443497 |
| | carboxylic acid | | | | | |
| 439 | 3,4,5-trihydroxy-6-[4-hydroxy-3-(3- | 550.14 | 10.386 | [M − H] − 1 | 44046888 ± | 14346146 ± |
| | phenylprop-2-enoyl)phenoxy]oxane-2- | | | | 12250562 | 2123539 |
| | carboxylic acid | | | | | |
| 440 | 7,14-dihydroxy-6-methoxy-4,12- | 562.14 | 11.722 | [M − H] − 1 | 199302 ± | 8211896 ± |
| | dimethyl-5-(3-methylbut-2-enoyl)-10- | | | | 25981 | 372731 |
| | oxo-2,9- | | | | | |
| | dioxatricyclo[9.4.0.0\xc2\xb3,\xe2\x81\ | | | | | |
| | xb8]pentadeca-1(11),3(8),4,6,12,14- | | | | | |
| | hexaene-15-carboxylic acid | | | | | |
| 441 | 6-[2-(benzoyloxy)-5-(prop-2-en-1- | 564.16 | 10.648 | [M − H] − 1 | 240806 ± | 196687 ± |
| | yl)phenoxy]-3,4,5-trihydroxyoxane-2- | | | | 7836 | 120183 |
| | carboxylic acid | | | | | |
| 442 | 3,4,5-trihydroxy-6-{2-[(1E)-3-(4- | 564.16 | 11.847 | [M − H] − 1 | 812681 ± | 54894976 ± |
| | methoxyphenyl)-3-oxoprop-1-en-1- | | | | 311959 | 17140906 |
| | yl]phenoxy}oxane-2-carboxylic acid | | | | | |
| 443 | 3,4,5-trihydroxy-6-{5-methoxy-2-[(2E)- | 564.16 | 11.781 | [M − H] − 1 | 879920 ± | 46776254 ± |
| | 3-phenylprop-2-enoyl]phenoxy}oxane-2- | | | | 197511 | 7914696 |
| | carboxylic acid | | | | | |
| 444 | 3,4,5-trihydroxy-6-{4-[(1E)-3-(4- | 564.16 | 11.532 | [M − H] − 1 | 1396695 ± | 54509330 ± |
| | methoxyphenyl)-3-oxoprop-1-en-1- | | | | 92231 | 19840841 |
| | yl]phenoxy}oxane-2-carboxylic acid | | | | | |
| 445 | 3,4,5-trihydroxy-6-{3-[(1E)-3-(4- | 564.16 | 11.953 | [M − H] − 1 | 1383091 ± | 10848077 ± |
| | methoxyphenyl)-3-oxoprop-1-en-1- | | | | 92167 | 3236227 |
| | yl]phenoxy}oxane-2-carboxylic acid | | | | | |
| 446 | Ketoprofen glucuronide | 564.16 | 11.213 | [M − H] − 1 | 2544589 ± | 4777722 ± |
| | | | | | 1554692 | 1434362 |
| 447 | 3,4,5-trihydroxy-6-{[8-methoxy-6-(3- | 570.17 | 4.615 | [M − H] − 1 | 228973368 ± | 600200 ± |
| | methylbut-2-en-1-yl)-2-oxo-2H- | | | | 15107157 | 74242 |
| | chromen-7-yl]oxy}oxane-2-carboxylic | | | | | |
| | acid | | | | | |
| 448 | simvastatin hydroxy acid | 570.32 | 11.389 | [M − H] − 1 | 34301301 ± | 166890 ± |
| | | | | | 5036658 | 11744 |
| 449 | Varanic acid | 570.35 | 11.427 | [M − H] − 1 | 3360794 ± | 99464 ± |
| | | | | | 262300 | 4778 |
| 450 | Formononetin 7-glucuronide | 580.15 | 3.987 | [M + H] + 1 | 77327 ± | 194116 ± |
| | | | | | 55313 | 131885 |
| 451 | 3,4,5-trihydroxy-6-[(8-methoxy-4-oxo-2- | 580.15 | 8.439 | [M − H] − 1 | 115818670 ± | 104279 ± |
| | phenyl-3,4-dihydro-2H-1-benzopyran-7- | | | | 14734095 | 6542 |
| | yl)oxy]oxane-2-carboxylic acid | | | | | |
| 452 | N-Stearoyl tyrosine | 581.37 | 12.27 | [M − H] − 1 | 12635191 ± | 120789 ± |
| | | | | | 2188071 | 2548 |
| 453 | 3alpha,7alpha,12alpha-trihydroxy-5beta- | 584.37 | 11.903 | [M − H] − 1 | 14617490 ± | 96856 ± |
| | cholestanate | | | | 1062188 | 3422 |
| 454 | 3,4,5-trihydroxy-6-{6-hydroxy-3-[3-(3- | 596.15 | 8.486 | [M − H] − 1 | 20764642 ± | 27169164 ± |
| | hydroxyphenyl)-3-oxoprop-1-en-1-yl]-2- | | | | 1225036 | 5774647 |
| | methoxyphenoxy}oxane-2-carboxylic | | | | | |
| | acid | | | | | |
| 455 | (3alpha,20R,24Z)-3-Hydroxy-21- | 606.39 | 9.368 | [M + H] + 1 | 98724208 ± | 4768874 ± |
| | oxoeupha-8,24-dien-26-oic acid | | | | 33043801 | 165596 |
| 456 | 23-Hydroxy-3-oxocycloart-24-en-26-oic | 604.38 | 12.257 | [M − H] − 1 | 7915874 ± | 95440 ± |
| | acid | | | | 2772630 | 9300 |
| 457 | Chenodeoxycholic acid 3-sulfate | 606.29 | 9.184 | [M − H] − 1 | 108202813 ± | 28217 ± |
| | | | | | 38912268 | 632 |
| 458 | Ursodeoxycholic acid 3-sulfate | 606.29 | 10.816 | [M − H] − 1 | 200146167 ± | 350728 ± |
| | | | | | 64217935 | 420289 |
| 459 | Chenodeoxycholic acid sulfate | 606.29 | 9.759 | [M − H] − 1 | 18347698 ± | 95308 ± |
| | | | | | 4837735 | 4174 |
| 460 | b“4-O-Methylepicatechin 7-O- | 614.16 | 7.92 | [M − H] − 1 | 65767168 ± | 92340 ± |
| | glucuronide” | | | | 3359931 | 17237 |
| 461 | 6-{5-[(E)-2-(2,4- | 622.20 | 10.042 | [M − H] − 1 | 1486863 ± | 171224 ± |
| | dihydroxyphenyl)ethenyl]-3-hydroxy-2- | | | | 459795 | 11362 |
| | (3-methylbut-2-en-1-yl)phenoxy}-3,4,5- | | | | | |
| | trihydroxyoxane-2-carboxylic acid | | | | | |
| 462 | 7-Sulfocholic acid | 622.28 | 9.981 | [M − H] − 1 | 21115252 ± | 339947 ± |
| | | | | | 1446294 | 25419 |
| 463 | Fexofenadine | 635.32 | 10.714 | [M − H] − 1 | 4092130 ± | 224486 ± |
| | | | | | 121573 | 4692 |
| 464 | 3,4,5-trihydroxy-6-({5,6,14-trimethoxy- | 640.18 | 10.132 | [M − H] − 1 | 83141191 ± | 132244 ± |
| | 8,17- | | | | 16073507 | 5392 |
| | dioxatetracyclo[8.7.0.0\xc2\xb2,\xe2\x81\ | | | | | |
| | xb7.0\xc2\xb9\xc2\xb9,\xc2\xb9\xe2\x81\ | | | | | |
| | xb6]heptadeca-2,4,6,11(16),12,14- | | | | | |
| | hexaen-4-yl}oxy)oxane-2-carboxylic | | | | | |
| | acid | | | | | |
| 465 | 4-{7-[(6-carboxy-3,4,5-trihydroxyoxan- | 648.12 | 12.011 | [M − H] − 1 | 337835 ± | 6657664 ± |
| | 2-yl)oxy]-3-hydroxy-5-sulfino-3,4- | | | | 43390 | 1417613 |
| | dihydro-2H-1-benzopyran-2-yl}-2- | | | | | |
| | hydroxybenzen-1-olate | | | | | |
| 466 | 3,3-Diiodothyronine | 658.93 | 7.666 | [M − H] − 1 | 778652 ± | 66407 ± |
| | | | | | 28870 | 38205 |
| 467 | 6-{[6-(3,4-dihydroxy-6-methyl-5- | 740.19 | 3.802 | [M + H] + 1 | 2510636 ± | 15222 ± |
| | oxooxan-2-yl)-5-hydroxy-2-(3- | | | | 93502 | 1211 |
| | methoxyphenyl)-4-oxo-4H-chromen-7- | | | | | |
| | yl]oxy}-3,4,5-trihydroxyoxane-2- | | | | | |
| | carboxylic acid | | | | | |

What is claimed is:

1. A quantitative detection method of multiple metabolic components in a biological sample, comprising performing derivatization treatment on the biological sample and then detecting the derivatized biological sample by liquid chromatography-mass spectrometry, wherein during derivatization treatment, 3-nitrophenylhydrazine is used as a derivatization reagent, and 1-(3-dimethylaminopropyl)-3-ethylcarbodiimide is used as a derivatization reaction catalyst; the biological sample is selected from urine, blood, cerebrospinal fluid, tissue, cells, saliva and fecal samples of a mammal; the multiple metabolic components in the biological sample detected by the quantitative detection method are selected from amino acid, phenol, phenyl or benzyl derivative, indole, organic acid, fatty acid, sugar, and bile acid and have different magnitudes in content;
the number of the multiple metabolic components in a biological sample detected by the quantitative detection method is 50 or more;
the liquid chromatography adopts a UPLC BEH C18 chromatographic column, the mobile phase includes: A: water containing 0.1% formic acid, B: acetonitrile containing 0.1% formic acid and isopropanol at a ratio of 1-2:1; and the gradient elution conditions: 0-1 min, 5% B; 1-5 min, 5-30% B; 5-9 min, 30-50% B; 9-12 min, 50-75% B; 12-15 min, 75-95% B; 15-16 min, 95-100% B; 16-18 min, 100% B; and 18-20 min, 5% B.

2. The detection method according to claim 1, wherein the number of the multiple metabolic components in a biological sample detected by the quantitative detection method is 100 or more.

3. The detection method according to claim 1, wherein the number of the multiple metabolic components in a biological sample detected by the quantitative detection method is 200 or more.

4. The detection method according to claim 1, wherein the number of the multiple metabolic components in a biological sample detected by the quantitative detection method is 600 or more.

5. The detection method according to claim 1, wherein the number of the multiple metabolic components in a biological sample detected by the quantitative detection method is 1000 or more.

6. The detection method according to claim 1, wherein scan parameters of the mass spectrometry are set as follows: S-lens RF level, 50; mass range, 100 to 1200 m/z; full MS resolution, 70,000; MS/MS resolution, 17,500; and NCE/stepped NCE is set at 10, 20, and 40 eV.

7. The detection method according to claim 1, wherein ESI ion source parameters of the mass spectrometry are set as follows: spray voltage, 3800 V; sheath gas flow rate, 40 for ESI+ and 5 for ESI−; capillary temperature, 320° C.; probe heater temperature, 350° C.; aux gas flow rate, 10.

8. The detection method according to claim 1, wherein the liquid chromatography-mass spectrometry is acquired using a Vanquish Flex UHPLC system coupled to a Q Extractive Focus Orbitrap mass spectrometer equipped with a heated electrospray ionization source.

9. The detection method according to claim 1, wherein the mobile phase includes: A: water containing 0.1% formic acid, B: acetonitrile containing 0.1% formic acid and isopropanol at a ratio of 7:3.

10. The detection method according to claim 1, comprising the following steps:
a) collecting a biological sample;
b) extracting the biological sample with a mixed solvent of methanol, chloroform, and water, performing centrifugation, and then taking a supernatant, namely a biological sample extract;
c) adding the same volume of a 3-nitrophenylhydrazine methanol solution and a 1-(3-dimethylaminopropyl)-3-ethylcarbodiimide pyridine solution into the biological sample extract obtained in b), and performing uniform vortex mixing and heating for derivatization, wherein the concentration of used 3-nitrophenylhydrazine is 100-320 mmol/L, the concentration of 1-(3-dimethylaminopropyl)-3-ethylcarbodiimide is 50-200 mmol/L, the reaction temperature is 20-60° C., and the reaction time is 10-120 minutes;
d) adding a carbon-13 labeled isotope internal standard solution obtained from the reaction of 3-nitrophenylhydrazine and 1-(3-dimethylaminopropyl)-3-ethylcarbodiimide into the derivatized biological sample extract obtained in c); and
e) adding a methanol-water mixed solution into the sample in d) for dilution, and determining amino acid, phenol, phenyl or benzyl derivative, indole, organic acid, fatty acid, sugar, and bile acid by liquid chromatography-mass spectrometry.

11. The detection method according to claim 10, wherein in step c), the concentration of used 3-nitrophenylhydrazine is 150-220 mmol/L, the concentration of 1-(3-dimethylaminopropyl)-3-ethylcarbodiimide is 80-120 mmol/L, the reaction temperature is 20-40° C., and the reaction time is 30-60 minutes.

12. The detection method according to claim 10, wherein when the biological sample is urine, blood, saliva or cerebrospinal fluid, a treatment method of the biological sample in step b) comprises: taking an appropriate amount of the biological sample, extracting the biological sample with a mixed solvent of cold methanol, chloroform and water at a volume ratio of 3:1:1, shaking the mixture for a few seconds, performing centrifugation on the sample at a rotation speed of 10000-20000 rpm at a low temperature for 5-15 minutes, and transferring a supernatant into an autosampler glass vial for subsequent derivatization treatment.

13. The detection method according to claim 10, wherein when the biological sample is a fecal sample, a treatment method of the biological sample in step b) comprises: freeze-drying the fecal sample; homogenizing an appropriate amount of the freeze-dried fecal sample with an appropriate amount of a mixed solvent of cold methanol, chloroform and water at a volume ratio of 3:1:1, performing centrifugation on the sample at a rotation speed of 10000-20000 rpm at a low temperature for 15-30 minutes, and transferring a supernatant into an autosampler glass vial for subsequent derivatization treatment.

14. The detection method according to claim 10, wherein when the biological sample is a tissue or cell sample, a treatment method of the biological sample in step b) comprises: adding an appropriate amount of a mixed solvent of cold methanol, chloroform and water at a volume ratio of 3:1:1 into the sample for homogenization, performing centrifugation on the sample at a rotation speed of 10000-20000 rpm at 4° C. for 15-30 minutes, and transferring a supernatant into an autosampler glass vial for subsequent derivatization treatment.

15. The detection method according to claim 10, wherein the volume ratio of methanol to water in step e) is 1:1.

16. The detection method according to claim 1, wherein the multiple metabolic components of the biological sample detected by the quantitative detection method are selected from the following multiple metabolites: fructose 1,6-diphosphate, 10Z-nonadecenoic acid, trans-11-octadecenoic acid, cis-11-octadecenoic acid, 12-dehydrocholic acid, 12-hydroxystearic acid, 12-ketolithocholic acid, 1-methylhistidine, 2,2-dimethylsuccinic acid, 2,3-diaminopropionic acid, glucoside 24-chenodeoxycholic acid, 2-butenoic acid, 2-oxoadipic acid, 2-methyl-4-pentenoic acid, 2-methyl-β-alanine, 2-methylbutyric acid, 2-methylhexanoic acid, 2-methylglutaric acid, 2-methylglutamic acid, 2-furoic acid, 2-hydroxy-2-methylbutyric acid, 2-hydroxy-3-methylbutyric acid, 2-hydroxybutyric acid, 2-hydroxycaproic acid, 2-hydroxycinnamic acid, 2-hydroxyphenylacetic acid, 2-hydroxyhippuric acid, 2-phenylpropionic acid, 2-phenylglycine, 3-(3-hydroxyphenyl)-3-hydroxypropionic acid, 3-(4-hydroxyphenyl)lactic acid, 3,4-dihydroxymandelic acid, 3,4-dihydroxyphenylpropionic acid, 3,4-dehydro-DL-proline, 3,5-diiodo-L-thyroxine, 30-cholic acid, 3-pyridineacetic acid, 3-indoleacrylic acid, 3-indolepropionic acid, 3-indoleacetamide, 3-oxyalanine, 3-aminosalicylic acid, 3-chloro-L-tyrosine, 3-methyl-2-oxobutyric acid, 3-methyl-2-oxovaleric acid, 3-methylindole, 3-methyladipic acid, 3-methylglutamic acid, 3-nitrotyrosine, sulfate 3-taurolithocholic acid, sulfate 3-lithocholic acid, 3-hydroxy-2-aminobenzoic acid, 3-hydroxybutyric acid, 3-hydroxypropionic acid, 3-hydroxyisovaleric acid, 3-hydroxyphenylacetic acid, 3-hydroxyhippuric acid, 3-dehydrocholic acid, 3-phenylbutyric acid, 4-methyl-2-oxovaleric acid, 4-methylhexanoic acid, 4-methoxyphenylacetic acid, 4-hydroxy-3-methoxymandelic acid, 4-hydroxycinnamic acid, 4-hydroxybenzoic acid, 4-hydroxyhippuric acid, 4-hydroxyphenyllactic acid, 4-hydroxyphenylpyruvic acid, 4-phenylbutyric acid, 5-aminolevulinic acid, 5-hydroxytryptophan, 5-hydroxytryptamine, 5-hydroxylysine, 6,7-diketolithocholic acid, 6-phosphogluconic acid, 6-ketolithocholic acid, 7,12-diketolithocholic acid, 7-ketolithocholic acid, 7-ketodeoxycholic acid, 9,11-conjugated linoleic acid, D-2-hydroxyglutaric acid, D-galactose, D-xylose, D-xylulose, D-fructose, D-ribose, D-ribose-5-phosphate, D-ribulose, D-ribulose-5-phosphate, D-mannose, D-glucose, D-maltose, L-2-aminobutyric acid, L-3-phenyllactic acid, L-alanine, L-serine, L-acetylcarnitine, L-lactic acid, L-allothreonine, L-cysteine, L-homoserine, L-homocysteine, L-homocitrulline, L-pipecolic acid, L-aspartic acid, L-asparagine, L-sorbose, L-lignic acid, L-norleucine, L-kynurenine, L-thyronine, L-arginine, L-histidine, L-valine, L-cystine, L-cystathionine, L-proline, L-tryptophan, L-threonine, L-phenylalanine, L-malic acid, L-methionine, L-glutamine, L-glutamic acid, L-lysine, L-tyrosine, L-arabinose, N-(3-phenylpropionyl)glycine, N-acetyl-L-alanine, N-acetyl-L-aspartic acid, N-acetyl-L-phenylalanine, N-acetyl-L-methionine, N-acetyl-L-tyrosine, N-acetylserine, N-acetyl-D-glucosamine, N-acetyl-L-phenylalanine, N-acetylmannosamine, N-acetylneuraminic acid, N-acetylhistidine, N-acetylhydroxytryptamine, N-acetyltryptophan, N-acetylglutamine, N-acetyllysine, N-acetylornithine, N-methylnicotinamide, N-phenylacetyl-glutamine, N-phenylacetylphenylalanine, S-adenosine homocysteine, α-D-glucose, α-lactose, α-linolenic acid, α-hydroxyisobutyric acid, α-ketoglutaric acid, α-muricholic acid, β-D-trehalose, β-alanine, β-ursocholic acid, β-muricholic acid, γ-L-glutamyl-L-alanine, γ-linolenic acid, γ-aminobutyric acid, ω-muricholic acid, butyric acid, trimethylamine nitroxide, adenosine triphosphate, malonic acid, propionic acid, acetoacetic acid, acetylcysteine, acetylglycine, acetylornithine, acetic acid, guanidine acetate, glycolic acid, lactulose, lactoylglutathione, heneicosanoic acid, cis-12-heneicosenoic acid, heptacosanoic acid, tricosanoic acid, cis-14-tricosenoic acid, docosanoic acid, docosatrienoic acid, cis-13,16-docosadienoic acid, docosapentaenoic acid, docosahexaenoic acid, docosatetraenoic acid, trans-13-docosaenoic acid, cis-13-docosaenoic acid, pentacosanoic acid, octacosanoic acid, hexacosanoic acid, tetracosanoic acid, eicosanoic acid, eicosatrienoic acid, eicosadienoic acid, eicosapentaenoic acid, trans-11-eicosenoic acid, cis-11-eicosenoic acid, cis-5-eicosenoic acid, cis-8-eicosenoic acid, dimethylglycine, adenosine diphosphate, linoleic acid, leucine, alloisoleucine, allolithocholic acid, allocholic acid, undecenoic acid, heptadecanoic acid, tridecanoic acid, nonadecanoic acid, nonadecadienoic acid, pentadecanoic acid, galactonic acid, galactitol, mecysteine, protocatechuic acid, apocholic acid, dehydrolithocholic acid, nordeoxycholic acid, trans-9-tetradecenoic acid, trans-aconitic acid, trans-4-hydroxyproline, trans-9-heptadecenoic acid, trans-9-pentadecenoic acid, trans-9-hexadecenoic acid, trans-linolenic acid, trans-cinnamic acid, elaidic acid, homocysteine, pyrrole-2-carboxylic acid, picolinic acid, indole, indole-3-methyl acetate, indole-3-carboxylic acid, indoleacetic acid, purine, azelaic acid, nonanoic acid, dopa, dopamine, melibiose, fumaric acid, acetaminophen, p-aminohippuric acid, p-cresol sulfate, symmetrical dimethylarginine, p-hydroxymandelic acid, p-hydroxyphenylacetic acid, homogentisic acid, adipic acid, pimelic acid, isobutyric acid, isoleucine, isovaleric acid, citric acid, isoursodeoxycholic acid, isohyodeoxycholic acid, isolithocholic acid, isocholic acid, isodeoxycholic acid, glutaric acid, glutaconic acid, valeric acid, mandelic acid, lauric acid, fructose-6-phosphate, citraconic acid, citric acid, citramalic acid, ribonolactone, ribonic acid, raffinose, palmitoleic acid, palmitic acid, norvaline, n-hydroxyphenylacetic acid, oxidized glutathione, aminoadipic acid, aminocaproic acid, salicyluric acid, oleic acid, trehalose, nicotinic acid, pyroglutamic acid, ursocholic acid, ursodeoxycholic acid, tauro-a-muricholic acid, tauro-b-muricholic acid, tauro-w-muricholic acid, tauroursodeoxycholic acid, taurohyocholic acid, taurohyodeoxycholic acid, taurolithocholic acid, taurocholic acid, taurodehydrocholic acid, taurochenodeoxycholic acid, hyocholic acid, hyodeoxycholic acid, succinylacetone, succinic acid, citrulline, glycodehydrocholic acid, glycolithocholic acid, glycodeoxycholic acid, glycine, glycochenodeoxycholic acid, glyceraldehyde, glyceraldehyde-3-phosphate, choline glycerophosphate, glycoursodeoxycholic acid, glycohyocholic acid, glycohyodeoxycholic acid, glycocholic acid, glyproline, glycyl-L-leucine, mannose-6-phosphoric acid, mannitol, methylmalonic acid, methylsuccinic acid, formic acid, capric acid, lithocholic acid, selenomethionine, thiamine, glycolithocholic sulfate, stearic acid, liothyronine, dihydroxyacetone phosphate, phosphoribosyl pyrophosphate, creatine phosphate, hydroxypyruvic acid, glycine hydroxyphenylacetate, cinnamic acid, sarcosine, carnosine, creatine, inositol, epinephrine, choline, cholic acid, dehydrocholic acid, demethylcholic acid, adenosine monophosphate, arachidonic acid, phenylpyruvic acid, phenethylamine, phenylpropionic acid, phenyllactic acid, benzamide, benzoic acid, oxalic acid, shikimic acid, glucaric acid, glucose-6-phosphate, glucose lactone, sebacic acid, ricinoleic acid, sucrose, methionine sulfoxide, itaconic acid, melatonin, glutathione, myristic acid, erythronic acid, erythrose, suberic acid, caprylic acid, phthalic acid, tartaric acid, tyramine, quinic acid, m-aminobenzoic acid, asymmetric dimethylarginine, tannic acid, cis-10,12-octadecadienoic acid, cis-12,15-heneicosadienoic acid, cis-12-tridecenoic acid, cis-15-tetracosenic acid, cis-2-hydroxycinnamic acid, cis-9-tetradecenoic acid, cis-10-heptadecenoic acid, cis-11-dodecenoic acid, cis-4-hydroxyproline, cis-5-dodecenoic acid, cis-7-hexadecenoic acid, cis-9-heptadecenoic acid, cis-aconitic acid, vanillic acid, hippuric acid, maleic acid, homovanillic acid, ornithine, guanosine monophosphate, guanosine triphosphate, anserine, chenodeoxycholic acid, maltotriose, maltitol, rhamnose and murideoxycholic acid.

17. A metabolic chip used in the detection method according to claim 1, comprising a chip carrier, a filter device and dry solid powder of a standard product and a quality control product, wherein the chip carrier is a microtiter plate, each well of the microtiter plate is provided with an independent filter device, and the powder obtained by dehydrating or freeze-drying solutions of the standard product and the quality control product is placed on the filter device in each well of the microtiter plate; the multiple metabolic components in the standard product are selected from amino acid, phenol, phenyl or benzyl derivative, indole, organic acid, fatty acid, sugar, and bile acid standard products; the multiple metabolic components in the quality control product are selected from amino acid, phenol, phenyl or benzyl derivative, indole, organic acid, fatty acid, sugar, and bile acid standard products corresponding to the standard products above;
the number of the multiple metabolic components in the standard product and the quality control product is 50 or more.

18. The metabolic chip according to claim 17, wherein the microtiter plate is selected from a 48-well plate, a 96-well plate and a 384-well plate, and the filter device is a filter membrane made of polyvinylidene fluoride, cellulose acetate, or nylon with a pore size of 0.20-0.45 micron.

19. The metabolic chip according to claim 17, wherein the multiple metabolic components in the standard product and the quality control product are selected from: fructose 1,6-diphosphate, 10Z-nonadecenoic acid, trans-11-octadecenoic acid, cis-11-octadecenoic acid, 12-dehydrocholic acid, 12-hydroxystearic acid, 12-ketolithocholic acid, 1-methylhistidine, 2,2-dimethylsuccinic acid, 2,3-diaminopropionic acid, glucoside 24-chenodeoxycholic acid, 2-butenoic acid, 2-oxoadipic acid, 2-methyl-4-pentenoic acid, 2-methyl-β-alanine, 2-methylbutyric acid, 2-methylhexanoic acid, 2-methylglutaric acid, 2-methylglutamic acid, 2-furoic acid, 2-hydroxy-2-methylbutyric acid, 2-hydroxy-3-methylbutyric acid, 2-hydroxybutyric acid, 2-hydroxycaproic acid, 2-hydroxycinnamic acid, 2-hydroxyphenylacetic acid, 2-hydroxyhippuric acid, 2-phenylpropionic acid, 2-phenylglycine, 3-(3-hydroxyphenyl)-3-hydroxypropionic acid, 3-(4-hydroxyphenyl)lactic acid, 3,4-dihydroxymandelic acid, 3,4-dihydroxyphenylpropionic acid, 3,4-dehydro-DL-proline, 3,5-diiodo-L-thyroxine, 30-cholic acid, 3-pyridineacetic acid, 3-indoleacrylic acid, 3-indolepropionic acid, 3-indoleacetamide, 3-oxyalanine, 3-aminosalicylic acid, 3-chloro-L-tyrosine, 3-methyl-2-oxobutyric acid, 3-methyl-2-oxovaleric acid, 3-methylindole, 3-methyladipic acid, 3-methylglutamic acid, 3-nitrotyrosine, sulfate 3-taurolithocholic acid, sulfate 3-lithocholic acid, 3-hydroxy-2-aminobenzoic acid, 3-hydroxybutyric acid, 3-hydroxypropionic acid, 3-hydroxyisovaleric acid, 3-hydroxyphenylacetic acid, 3-hydroxyhippuric acid, 3-dehydrocholic acid, 3-phenylbutyric acid, 4-methyl-2-oxyvaleric acid, 4-methylhexanoic acid, 4-methoxyphenylacetic acid, 4-hydroxy-3-methoxymandelic acid, 4-hydroxycinnamic acid, 4-hydroxybenzoic acid, 4-hydroxyhippuric acid, 4-hydroxyphenyllactic acid, 4-hydroxyphenylpyruvic acid, 4-phenylbutyric acid, 5-aminolevulinic acid, 5-hydroxytryptophan, 5-hydroxytryptamine, 5-hydroxylysine, 6,7-diketolithocholic acid, 6-phosphogluconic acid, 6-ketolithocholic acid, 7,12-diketolithocholic acid, 7-ketolithocholic acid, 7-ketodeoxycholic acid, 9,11-conjugated linoleic acid, D-2-hydroxyglutaric acid, D-galactose, D-xylose, D-xylulose, D-fructose, D-ribose, D-ribose-5-phosphate, D-ribulose, D-ribulose-5-phosphate, D-mannose, D-glucose, D-maltose, L-2-aminobutyric acid, L-3-phenyllactic acid, L-alanine, L-serine, L-acetylcarnitine, L-lactic acid, L-allothreonine, L-cysteine, L-homoserine, L-homocysteine, L-homocitrulline, L-pipecolic acid, L-aspartic acid, L-asparagine, L-sorbose, L-lignic acid, L-norleucine, L-kynurenine, L-thyronine, L-arginine, L-histidine, L-valine, L-cystine, L-cystathionine, L-proline, L-tryptophan, L-threonine, L-phenylalanine, L-malic acid, L-methionine, L-glutamine, L-glutamic acid, L-lysine, L-tyrosine, L-arabinose, N-(3-phenylpropionyl)glycine, N-acetyl-L-alanine, N-acetyl-L-aspartic acid, N-acetyl-L-phenylalanine, N-acetyl-L-methionine, N-acetyl-L-tyrosine, N-acetylserine, N-acetyl-D-glucosamine, N-acetyl-L-phenylalanine, N-acetylmannosamine, N-acetylneuraminic acid, N-acetylhistidine, N-acetylhydroxytryptamine, N-acetyltryptophan, N-acetylglutamine, N-acetyllysine, N-acetylornithine, N-methylnicotinamide, N-phenylacetyl-glutamine, N-phenylacetylphenylalanine, S-adenosine homocysteine, α-D-glucose, α-lactose, α-linolenic acid, α-hydroxyisobutyric acid, α-ketoglutaric acid, α-muricholic acid, β-D-trehalose, β-alanine, β-ursocholic acid, β-muricholic acid, γ-L-glutamyl-L-alanine, γ-linolenic acid, γ-aminobutyric acid, ω-muricholic acid, butyric acid, trimethylamine nitroxides, adenosine triphosphate, malonic acid, propionic acid, acetoacetic acid, acetylcysteine, acetylglycine, acetylornithine, acetic acid, guanidine acetate, glycolic acid, lactulose, lactoylglutathione, heneicosanoic acid, cis-12-heneicosenoic acid, heptacosanoic acid, tricosanoic acid, cis-14-tricosenoic acid, docosanoic acid, docosatrienoic acid, cis-13,16-docosadienoic acid, docosapentaenoic acid, docosahexaenoic acid, docosatetraenoic acid, trans-13-docosaenoic acid, cis-13-docosaenoic acid, pentacosanoic acid, octacosanoic acid, hexacosanoic acid, tetracosanoic acid, eicosanoic acid, eicosatrienoic acid, eicosadienoic acid, eicosapentaenoic acid, trans-11-eicosenoic acid, cis-11-eicosenoic acid, cis-5-eicosenoic acid, cis-8-eicosenoic acid, dimethylglycine, adenosine diphosphate, linoleic acid, leucine, alloisoleucine, allolithocholic acid, allocholic acid, undecenoic acid, heptadecanoic acid, tridecanoic acid, nonadecanoic acid, nonadecadienoic acid, pentadecanoic acid, galactonic acid, galactitol, mecysteine, protocatechuic acid, apocholic acid, dehydrolithocholic acid, nordeoxycholic acid, trans-9-tetradecenoic acid, trans-aconitic acid, trans-4-hydroxyproline, trans-9-heptadecenoic acid, trans-9-pentadecenoic acid, trans-9-hexadecenoic acid, trans-linolenic acid, trans-cinnamic acid, elaidic acid, homocysteine, pyrrole-2-carboxylic acid, picolinic acid, indole, indole-3-methyl acetate, indole-3-carboxylic acid, indoleacetic acid, purine, azelaic acid, nonanoic acid, dopa, dopamine, melibiose, fumaric acid, acetaminophen, p-aminohippuric acid, p-cresol sulfate, symmetrical dimethylarginine, p-hydroxymandelic acid, p-hydroxyphenylacetic acid, homogentisic acid, adipic acid, pimelic acid, isobutyric acid, isoleucine, isovaleric acid, citric acid, isoursodeoxycholic acid, isohyodeoxycholic acid, isolithocholic acid, isocholic acid, isodeoxycholic acid, glutaric acid, glutaconic acid, valeric acid, mandelic acid, lauric acid, fructose-6-phosphate, citraconic acid, citric acid, citramalic acid, ribonolactone, ribonic acid, raffinose, palmitoleic acid, palmitic acid, norvaline, n-hydroxyphenylacetic acid, oxidized glutathione, aminoadipic acid, aminocaproic acid, salicyluric acid, oleic acid, trehalose, nicotinic acid, pyroglutamic acid, ursocholic acid, ursodeoxycholic acid, tauro-a-muricholic acid, tauro-b-muricholic acid, tauro-w-muricholic acid, tauroursodeoxycholic acid, taurohyocholic acid, taurohyodeoxycholic acid, taurolithocholic acid, taurocholic acid, taurodehydrocholic acid, taurochenodeoxycholic acid, hyocholic acid, hyodeoxycholic acid, succinylacetone, succinic acid, citrulline, glycodehydrocholic acid, glycolithocholic acid, glycodeoxycholic acid, glycine, glycochenodeoxycholic acid, glyceraldehyde, glyceraldehyde-3-phosphate, choline glycerophosphate, glycoursodeoxycholic acid, glycohyocholic acid, glycohyodeoxycholic acid, glycocholic acid, glyproline, glycyl-L-leucine, mannose-6-phosphoric acid, mannitol, methylmalonic acid, methylsuccinic acid, formic acid, capric acid, lithocholic acid, selenomethionine, thiamine, glycolithocholic sulfate, stearic acid, liothyronine, dihydroxyacetone phosphate, phosphoribosyl pyrophosphate, creatine phosphate, hydroxypyruvic acid, glycine hydroxyphenylacetate, cinnamic acid, sarcosine, carnosine, creatine, inositol, epinephrine, choline, cholic acid, dehydrocholic acid, demethylcholic acid, adenosine monophosphate, arachidonic acid, phenylpyruvic acid, phenethylamine, phenylpropionic acid, phenyllactic acid, benzamide, benzoic acid, oxalic acid, shikimic acid, glucaric acid, glucose-6-phosphate, glucose lactone, sebacic acid, ricinoleic acid, sucrose, methionine sulfoxide, itaconic acid, melatonin, glutathione, myristic acid, erythronic acid, erythrose, suberic acid, caprylic acid, phthalic acid, tartaric acid, tyramine, quinic acid, m-aminobenzoic acid, asymmetric dimethylarginine, tannic acid, cis-10,12-octadecadienoic acid, cis-12,15-heneicosadienoic acid, cis-12-tridecenoic acid, cis-15-tetracosenic acid, cis-2-hydroxycinnamic acid, cis-9-tetradecenoic acid, cis-10-heptadecenoic acid, cis-11-dodecenoic acid, cis-4-hydroxyproline, cis-5-dodecenoic acid, cis-7-hexadecenoic acid, cis-9-heptadecenoic acid, cis-aconitic acid, vanillic acid, hippuric acid, maleic acid, homovanillic acid, ornithine, guanosine monophosphate, guanosine triphosphate, anserine, chenodeoxycholic acid, maltotriose, maltitol, rhamnose and murideoxycholic acid.

20. A use method of the metabolic chip according to claim 17, comprising the following steps:
a) collecting a biological sample;
b) according to the sample type, preparing a corresponding biological sample extract, when the biological sample is urine, blood, saliva or cerebrospinal fluid, a treatment method of the biological sample comprises: taking an appropriate amount of the biological sample, extracting the biological sample with a mixed solvent of cold methanol, chloroform and water at a volume ratio of 3:1:1, shaking the mixture for a few seconds, performing centrifugation on the sample at a rotation speed of 10000-20000 rpm at a low temperature for 5-15 minutes, and transferring a supernatant into an autosampler glass vial for subsequent derivatization treatment;
c) adding the prepared biological sample extract into each well of the metabolic chip in an equal amount, adding the same volume of a 3-nitrophenylhydrazine methanol solution and a 1-(3-dimethylaminopropyl)-3-ethylcarbodiimide pyridine solution into each well, and performing uniform vortex mixing and heating for derivatization, wherein the concentration of used 3-nitrophenylhydrazine is 100-320 mmol/L, the concentration of 1-(3-dimethylaminopropyl)-3-ethylcarbodiimide is 50-200 mmol/L, the reaction temperature is 20-60° C., and the reaction time is 10-120 minutes;
d) adding a carbon-13 labeled isotope internal standard solution obtained from the reaction of 3-nitrophenylhydrazine and 1-(3-dimethylaminopropyl)-3-ethylcarbodiimide into the derivatized biological sample extract obtained in c); and
e) adding a methanol-water mixed solution into each well in the metabolic chip in d) for dilution, placing the metabolic chip in a tandem mass spectrometer for determination of amino acid, phenol, phenyl or benzyl derivative, indole, organic acid, fatty acid, sugar, and bile acid by liquid chromatography-mass spectrometry, and calculating concentrations of target metabolites in the sample based on results.
